(19) United States
(12) Patent Application Publication
(10) Pub. No.: US 2014/0228286 A1
(43) Pub. Date: Aug. 14, 2014

(54) SPECIFIC PDE4B-INHIBITORS FOR THE TREATMENT OF DIABETES MELLITUS

(71) Applicants: Gerd Luippold, Warthausen-Birkenhard (DE); Peter NICKOLAUS, Warthausen (DE); Ruediger STREICHER, Biberach an der Riss (DE)

(72) Inventors: Gerd Luippold, Warthausen-Birkenhard (DE); Peter NICKOLAUS, Warthausen (DE); Ruediger STREICHER, Biberach an der Riss (DE)

(73) Assignee: BOEHRINGER INGELHEIM INTERNATIONAL GMBH, Ingelheim am Rhein (DE)

(21) Appl. No.: 14/178,321

(22) Filed: Feb. 12, 2014

(30) Foreign Application Priority Data
Feb. 14, 2013 (EP) 13155200.2

(51) Int. Cl. A61K 31/519; A61K 45/06

(52) U.S. Cl. CPC A61K 31/519; A61K 45/06
USPC 514/6.5; 514/260.1; 514/7.2

(57) ABSTRACT

A method of treating diabetes mellitus or a microvascular or macrovascular complication of diabetes mellitus in a patient in need thereof, the method comprising administering to the patient a compound of formula 1

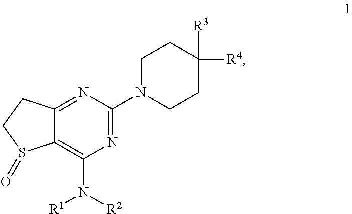

wherein R¹, R², R³, and R⁴ are as defined in claim 1.

### RELATED APPLICATION

This application claims priority to European Patent Application No. 13155200.2, filed Feb. 14, 2013, the contents of which are hereby incorporated by reference in its entirety.

### SUMMARY OF THE INVENTION

The invention relates to compounds of formula 1 for their use for the treatment of diabetes mellitus or for the treatment of a microvascular or macrovascular complication of diabetes mellitus

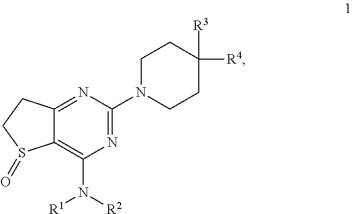

wherein
R¹, R², R³ and R⁴ are defined as summarized in claim 1.

Further the invention relates to the use of compounds of the above formula 1 for the manufacture of a medicament for the treatment of diabetes mellitus.

### BACKGROUND OF THE INVENTION

### Diabetes Mellitus

Diabetes mellitus is on the rise worldwide and is considered to be at an epidemic level by the World Health Organization. The clinical manifestation and progression of diabetes often vary considerably between countries and commonly between ethnic groups in the same country. Currently diabetes affects 151 million people worldwide and an estimate 300 million people in 2025. There are two main forms of diabetes.

Type 1 (insulin-dependent diabetes mellitus, IDDM) is due primarily to autoimmune-mediated destruction of pancreatic β-cells, resulting in absolute insulin deficiency. It is the second most common chronic disease of children. By contrast, type 2 diabetes (non-insulin-dependent diabetes mellitus, NIDDM) is characterized by insulin-resistance and inadequate insulin secretion. A significant fraction of individuals originally diagnosed with type 2 diabetes evolve with time to a type 1 state, which is defined as exhibiting anti-β-cell autoimmunity.

Because genetic factors contribute to the development of diabetes, the disease displays a strong familial aggregation. Although there are monogenic syndromes of insulin resistance, in which a definite gene has been identified as the cause of insulin resistance, these are relative rare. The more common presentation of diabetes appears to be polygenic. Additionally, behavioral- and lifestyle-related risk factors exist. Type 2 diabetes is increasingly common primarily because of increases in the prevalence of a sedentary lifestyle and obesity. One of the major arguments for the role of behavioral factors in the etiology of diabetes has been the rapid increase in the prevalence and incidence of the disease in populations undergoing rapid westernization. The westernization transition is usually accompanied by increases in obesity, decreases in physical activity and alterations in dietary intake toward more calories, fat and non-complex carbohydrates.

Plasma glucose concentrations are normally maintained within a fairly narrow range despite wide fluctuations in the body's supply (e.g. meals) and demand (e.g. exercise) for nutrients. After an overnight fast, insulin-independent tissues, the brain (50%) and splanchnic organs (25%), account for most of the total body glucose disposal. Insulin-dependent tissues, adipose tissue and primarily skeletal muscles, are responsible for the remaining 25% of glucose utilization. This basal glucose uptake is precisely matched by the release of glucose from the liver. In response to hyperglycemia after a meal, pancreatic insulin secretion is stimulated and the combination of hyperinsulinemia plus hyperglycemia promotes glucose uptake (by splanchnic and peripheral, primarily muscle tissues) and suppresses hepatic glucose production. It follows, therefore, that defects at the level of the β-cell, muscle and liver can lead to the development of glucose intolerance and diabetes mellitus. All the abnormalities in diabetes basically result from an imbalance between insulin sensitivity and insulin secretion. The initial stage of diabetes is characterized by impaired glucose tolerance and postprandial hyperglycemia. As the disease progresses, fasting hyperglycemia is observed.

The earliest detectable abnormality in NIDDM is an impairment in the body's ability to respond to insulin. Because the pancreas is able to appropriately augment its secretion of insulin to offset the insulin resistance, glucose tolerance remains normal. With time, however, the beta-cell fails to maintain its high rate of insulin secretion and the insulin resistance leads to the development of impaired glucose tolerance and eventually overt diabetes mellitus. The cause of pancreatic “exhaustion” remains unknown. Insulin resistance in NIDDM involves both hepatic and peripheral tissues. In response to both endogenously secreted or exogenously administered insulin, hepatic glucose production fails to suppress normally and muscle glucose uptake is diminished. The accelerated rate of hepatic glucose output is due mainly to augmented gluconeogenesis. In muscle many cellular defects in insulin action have been described including impaired insulin-receptor tyrosine kinase activity, diminished glucose transport and reduced glycogen synthase and pyruvate dehydrogenase activities. The abnormalities account for disturbances in the two major intracellular pathways of glucose disposal, glycogen synthesis and glucose oxidation. In the earliest stages of NIDDM, the major defect involves the inability of insulin to promote glucose uptake and storage as glycogen. Other potential mechanisms that have been put forward to explain the glucose intolerance include increased levels of free fatty acids, chronic low-grade activation of the immune system (increased levels of TNFalpha and IL6), altered skeletal muscle blood flow, increased conversion of amylin to its insoluble amyloid form and glucose toxicity.

Diabetes is associated with a variety of physiologic disorders such as hypertension and dyslipidemia. Diabetes also increases the risk of microvascular (coronary artery disease, stroke, amputation) and microvascular (blindness, renal failure, neuropathies) diseases. Myocardial infarction, stroke or renal failure are the cause of death for more than 70% of diabetes patients. The huge mortality and debilitating neuropathies associated with diabetes underline the importance of active medical intervention.

There are several ways to counteract diabetes. The first is lifestyle adjustments aimed at improving endogenous insulin sensitivity. This can be achieved by increased physical activity and bodyweight reduction with diet and behavioral modification. Unfortunately, most people with non-insulin-dependent diabetes mellitus never receive sufficient nutritional education or are not capable of complying with a strict diet regimen.

Another therapeutic way involves increasing insulin availability by the administration of exogenous insulin, insulin analogues and insulin secretagogues such as sulphonylureas. The primary mode of action of sulphonylureas is through the depolarization of the pancreatic β-cells by blocking the ATP-dependent potassium channels and causing an influx of calcium ions, which stimulate insulin secretion. The most frequently encountered adverse effect of insulin, insulin analogues and insulin secretagogues is hypoglycemia. Body weight gain can also be a concern, because insulin not only increases uptake of blood glucose but also promotes the synthesis and storage of lipids.

Biguanides, of which metformin is the most commonly used, also have proven to be effective anti-hyperglycemic agents. Metformin reduces hepatic gluconeogenesis and basal hepatic glucose output. Its most serious adverse effect is lactic acidosis. Other common adverse effects of metformin are nausea and anorexia. Oral antidiabetics such as sulphonylureas and metformin as monotherapy or in combination have been shown to decrease fasting plasma glucose levels, but postprandial hyperglycemia persists in more than 60% of patients and probably accounts for sustained increases of hemoglobin levels.

Alpha-Glucosidase inhibitors, e.g. acarbose and miglitol, primarily target postprandial hyperglycemia. The therapy of diabetes mellitus with alpha-glucosidase inhibitors is based on a delayed intestinal degradation of starch and sucrose. These carbohydrates must be hydrolyzed by alpha-glucosidases to monosaccharides before they can be transported through the mucosa of the small intestine. The reversible inhibition of the brush border glucosidases results in redistribution of carbohydrate absorption from the upper portion of the gut to a more extended surface area covering the whole length of the small intestine. This is accompanied by a delayed absorption of monosaccharides and a decrease in the postprandial elevation of blood glucose. Common adverse effects of alpha-Glucosidase inhibitors are symptoms of carbohydrate malabsorption and gastrointestinal discomfort.

Another class of antidiabetic drugs are thiazolidinediones, such as rosiglitazone and pioglitazone, which are insulin sensitizers and act through activation of peroxisome proliferator-activated receptor y (PPARy). PPARy is mainly expressed in adipose tissues, plays an important role in adipogenesis and modifies fatty acid synthesis and storage. Binding of rosiglitazone to PPARy results in reduced endogenous glucose production and increased blood glucose uptake. It increases the sensitivity of skeletal muscle, liver and adipose tissues to insulin. Improvements in glucose metabolism with rosiglitazone treatment are closely correlated with decreased plasma free fatty acid metabolism. The stimulation by rosiglitazone of PPARy in adipose tissue and subsequent adipocyte differentiation results in the generation of more, but smaller, adipocytes which are more insulin sensitive and produce less free fatty acid, TNFalpha and leptin. Common adverse effects of rosiglitazone are anemia, edema and increased body weight.

Diabetes mellitus is often associated with both macrovascular complications (involving large blood vessels) and microvascular complications (involving small blood vessels), resulting in organ and tissue damage in approximately one third to one half of people with diabetes. Examples for frequent macrovascular complications of diabetes mellitus are myocardial infarct, acute coronary syndrome, unstable angina pectoris, stable angina pectoris, peripheral arterial occlusive disease, cardiomyopathy, heart failure, heart rhythm disorders, vascular restenosis and stroke. Examples for frequent microvascular complications of diabetes mellitus are diabetic retinopathy, diabetic nephropathy, diabetic neuropathy, and consequently diabetic foot and diabetic ulcer.

Diabetic retinopathy, the most common diabetic eye disease, occurs when blood vessels in the retina change. Sometimes these vessels swell and leak fluid or even close off completely. In other cases, abnormal new blood vessels grow on the surface of the retina. Diabetic retinopathy usually affects both eyes. People who have diabetic retinopathy often don't notice changes in their vision in the disease's early stages. But as it progresses, diabetic retinopathy usually causes vision loss that in many cases cannot be reversed.

Diabetic nephropathy, also known as Kimmelstiel-Wilson syndrome, or nodular diabetic glomerulosclerosis and intercapillary glomerulonephritis, is a progressive kidney disease caused by angiopathy of capillaries in the kidney glomeruli. It is characterized by nephrotic syndrome and diffuse glomerulosclerosis. It is due to longstanding diabetes mellitus, and is a prime indication for dialysis in many Western countries.

Kidney failure provoked by glomerulosclerosis leads to fluid filtration deficits and other disorders of kidney function. There is an increase in blood pressure (hypertension) and fluid retention in the body, further, a reduced plasma oncotic pressure causes edema. Other complications may be arteriosclerosis of the renal artery and proteinuria.

Throughout its early course, diabetic nephropathy has no symptoms. They develop in late stages and may be a result of excretion of high amounts of protein in the urine or due to renal failure:
edema: swelling, usually around the eyes in the mornings; later, general body swelling may result, such as swelling of the legs
foamy appearance or excessive frothing of the urine (caused by the proteinuria)
unintentional weight gain (from fluid accumulation)
anorexia (poor appetite)
nausea and vomiting
malaise (general ill feeling)
fatigue
headache
frequent hiccups

The first laboratory abnormality is a positive microalbuminuria test. Most often, the diagnosis is suspected when a routine urinalysis of a person with diabetes shows too much protein in the urine (proteinuria). The urinalysis may also show glucose in the urine, especially if blood glucose is poorly controlled. Serum creatinine and BUN may increase as kidney damage progresses.

A kidney biopsy confirms the diagnosis, although it is not always necessary if the case is straightforward, with a documented progression of proteinuria over time and presence of diabetic retinopathy on examination of the retina of the eyes.

Diabetic neuropathies are a family of nerve disorders caused by diabetes. People with diabetes can, over time, develop nerve damage throughout the body. Some people with nerve damage have no symptoms. Others may have symptoms such as pain, tingling, or numbness—loss of feeling—in the hands, arms, feet, and legs. Nerve problems can occur in every organ system, including the digestive tract, heart, and sex organs.

About 60 to 70 percent of people with diabetes have some form of neuropathy. People with diabetes can develop nerve problems at any time, but risk rises with age and longer duration of diabetes. The highest rates of neuropathy are among people who have had diabetes for at least 25 years. Diabetic neuropathies also appear to be more common in people who have problems controlling their blood glucose, also called blood sugar, as well as those with high levels of blood lipids and blood pressure and those who are overweight.

### PDE4-Inhibitors

PDE4-inhibitors are known to have a wide range of applications in the therapeutic field. Examples include respiratory or gastrointestinal diseases or complaints, inflammatory diseases of the joints, skin or eyes, cancers, and also diseases of the peripheral or central nervous system.

Particularly PDE4-inhibitors may be used in the prevention and treatment of diseases of the airways and of the lung which are accompanied by increased mucus production, inflammations and/or obstructive diseases of the airways. Examples include acute, allergic or chronic bronchitis, chronic obstructive bronchitis (COPD), coughing, pulmonary emphysema, allergic or non-allergic rhinitis or sinusitis, chronic rhinitis or sinusitis, asthma, idiopathic pulmonary fibrosis, alveolitis, Farmer's disease, hyperreactive airways, infectious bronchitis or pneumonitis, pediatric asthma, bronchiectasis, pulmonary fibrosis, ARDS (acute adult respiratory distress syndrome), bronchial edema, pulmonary edema, bronchitis, pneumonia or interstitial pneumonia triggered by various causes, such as aspiration, inhalation of toxic gases, or bronchitis, pneumonia or interstitial pneumonia as a result of heart failure, irradiation, chemotherapy, cystic fibrosis or mucoviscidosis, or alpha1-antitrypsin deficiency.

Also deserving special mention is the treatment of inflammatory diseases of the gastrointestinal tract by PDE4-inhibitors. Examples include acute or chronic inflammatory changes in gall bladder inflammation, Crohn's disease, ulcerative colitis, inflammatory pseudopolyps, juvenile polyps, colitis cystica profunda, pneumatosis cystoides intestinales, diseases of the bile duct and gall bladder, e.g. gallstones and conglomerates, for the treatment of inflammatory diseases of the joints such as rheumatoid arthritis or inflammatory diseases of the skin and eyes.

PDE4-inhibitors are also suited for the treatment of cancers. Examples include all forms of acute and chronic leukemias such as acute lymphatic and acute myeloid leukemia, chronic lymphatic and chronic myeloid leukemia as well as bone tumors such as e.g. osteosarcoma and all kinds of gliomas such as e.g. oligodendroglioma and glioblastoma.

Furthermore, PDE4-inhibitors are known for their therapeutic potential in the treatment of diseases of the peripheral or central nervous system. Examples of these include depression, bipolar or manic depression, acute and chronic anxiety states, schizophrenia, Alzheimer's disease, Parkinson's disease, acute and chronic multiple sclerosis or acute and chronic pain as well as injuries to the brain caused by stroke, hypoxia or craniocerebral trauma.

PDE4-inhibitors are also known for their suitability for the treatment of inflammatory or obstructive diseases of the upper and lower respiratory tract including the lungs, such as for example allergic rhinitis, chronic rhinitis, bronchiectasis, cystic fibrosis, idiopathic pulmonary fibrosis, fibrosing alveolitis, COPD, chronic bronchitis, chronic sinusitis, asthma, Crohn's disease, ulcerative colitis, particularly COPD, chronic bronchitis and asthma.

### PRIOR ART

U.S. Pat. No. 3,318,881 and BE 663693 disclose the preparation of piperazino-dihydrothieno-[3,2-d]pyrimidines which have cardiovascular and sedative properties. However, WO 2006/111549 and WO 2007/118793 both disclose dihydrothienopyrimidinesulphoxides which are substituted by piperazine instead of piperidine as PDE4-inhibitors for the treatment of inflammatory or obstructive pulmonary diseases such as asthma and COPD.

WO 2009/050248 and PCT/EP2012066104 both disclose piperidino-dihydrothienopyrimidinesulphoxides as PDE4-inhibitors for the treatment of inflammatory or obstructive pulmonary diseases such as asthma and COPD.

U.S. Pat. No. 8,017,633 discloses the use of the PDE4-inhibitor Roflumilast for the treatment of diabetes mellitus.

WO 08028914 discloses the use of (2R,4aR, 10bR)-6-(2.6-dimethoxy-pyridin-3-yl)-9-ethoxy-8-methoxy-1,2,3,4,4a,10b-hexahydrophenanthrin-2-ol for the treatment of diabetes mellitus type I or type II.

### DESCRIPTION OF THE INVENTION

Surprisingly it has been found that piperidino-dihydrothienopyrimidinesulphoxides of formula 1, wherein R¹, R², R³, and R⁴ have the meanings as stated in claim 1 are particularly suitable for the treatment of diabetes mellitus and for the treatment of diabetes-accompanying diseases such as diabetic retinopathy and diabetic nephropathy.

The present invention therefore relates to compounds of formula 1 for their use for the treatment of diabetes mellitus or for the treatment of a microvascular or macrovascular complication of diabetes mellitus

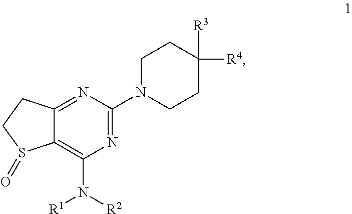

wherein
R¹ denotes H,
R² is H or a group selected from among C₁₋₁₀-alkyl and C₂₋₆-alkenyl, which may optionally be substituted by one or more groups selected from halogen and C₁₋₃-fluoroalkyl or which may optionally be substituted by one or more groups selected from among OR^{2.1}, COOR^{2.1}, CONR^{2.2}R^{2.3}, SR^{2.1}, SO—R^{2.1}, SO₂—R^{2.1}, C₆₋₁₀-aryl, het, hetaryl, a mono- or bicyclic —C₃₋₁₀-cycloalkyl, CH₂—NR^{2.2}R^{2.3} and NR^{2.2}R^{2.3}, which in turn may optionally be substituted by one or more groups selected from among OH, halogen, OR^{2.1}, oxo, CF₃, CHF₂, CH₂F, C₁₋₆-alkyl, C₁₋₆-alkanol, C₆₋₁₀-aryl, COOR^{2.1}, CH₂—NR^{2.2}R^{2.3} and NR^{2.2}R^{2.3},
   wherein
het denotes a three- to eleven-membered, mono- or bicyclic, saturated or partially saturated, optionally anellated or optionally bridged heterocycle is, which contains 1, 2, 3 or 4 heteroatoms selected independently of one another from among N, S or O contains, and wherein
hetaryl is a five- to ten-membered, mono- or bicyclic, optionally anellated heteroaryl, which contains 1, 2, 3 or 4 heteroatoms selected independently of one another from among N, S or O, and wherein
cycloalkyl may be saturated or partially saturated,
wherein R^{2.1} is H or is a group selected from among C₁₋₆-alkyl, C₁₋₆-alkanol, C₁₋₃-haloalkyl, mono- or bicyclic, —C₃₋₁₀-cycloalkyl, C₆₋₁₀-aryl-C₁₋₆-alkylene, hetaryl-C₁₋₆-alkylene, het-C₁₋₆-alkylene, C₃₋₁₀-cycloalkyl-C₁₋₆-alkylene, a mono- or bicyclic C₆₋₁₀-aryl, heteroaryl and a-het, which may optionally be substituted by one or more groups selected from among OH, O—(C₁₋₃-alkyl), halogen, C₁₋₆-alkyl and C₆₋₁₀-aryl,
wherein R^{2.2} and R^{2.3} independently of one another denote H or a group selected from among C₁₋₆-alkyl, mono- or bicyclic C₃₋₁₀ cycloalkyl, C₆₋₁₀-aryl-C₁₋₆-alkylene, hetaryl-C₁₋₆-alkylene, mono- or bicyclic C₆₋₁₀-aryl, het, hetaryl, CO—NH₂, CO—NHCH₃, —CO—N(CH₃)₂, SO₂—(C₁-C₂-alkyl), CO—R^{2.1} and COOR^{2.1}, which may optionally be substituted by one or more groups selected from among OH, halogen, C₁₋₆-alkyl, C₆₋₁₀-aryl and COOR^{2.1}, or
R² denotes a mono- or polycyclic C₃₋₁₀ cycloalkyl, which may optionally be bridged one or more times via C₁₋₃-alkyl groups and which may optionally be substituted by a group selected from among branched or unbranched C₁₋₆-alkanol, C₁₋₃-fluoroalkyl, C₁₋₃-alkylene-OR^{2.1}, OR^{2.1}, COOR^{2.1}, —SO₂—NR^{2.2}R^{2.3}, het, —NH—CO—O—(C₁₋₆-alkyl), —NH—CO—(C₁₋₆-alkyl), —NH—CO—O—(C₆₋₁₀-aryl), —NH—CO—(C₆₋₁₀-aryl), —NH—CO—O-hetaryl, —NH—CO-hetaryl, —NH—CO—O—(C₁₋₃-alkylene)-(C₆₋₁₀-aryl), —NH—CO—(C₁₋₃-alkylene)-(C₆₋₁₀-aryl), —N(C₁₋₃-alkyl)-CO—(C₁₋₆-alkyl), —N(C₁₋₃-alkyl)-CO—O—(C₆₋₁₀-aryl), —N(C₁₋₃-alkyl)-CO—(C₆₋₁₀-aryl), —N(C₁₋₃-alkyl)-CO—O-hetaryl, —N(C₁₋₃-alkyl)-CO-hetaryl, —N(C₁₋₃-alkyl)-CO—O—(C₁₋₃-alkylene)-(C₆₋₁₀-aryl), —N(C₁₋₃-alkyl)-CO—(C₁₋₃-alkylene)-(C₆₋₁₀-aryl), C₆₋₁₀-aryl, C₁₋₆-alkyl, C₆₋₁₀-aryl-C₁₋₆-alkylene, hetaryl-C₁₋₆-alkylene, mono- or bicyclic C₃₋₁₀ cycloalkyl and NR^{2.2}R^{2.3}, which may optionally be substituted by one or more groups selected from among OH, OR^{2.1}, oxo, halogen, CF₃, CHF₂, CH₂F, C₁₋₆-alkyl, C₆₋₁₀-aryl and NR^{2.2}R^{2.3}, or
R² denotes a mono- or polycyclic C₆₋₁₀-aryl, which may optionally be substituted by OH, SH or halogen or by one or more groups selected from among OR^{2.1}, COOR^{2.1}, NR^{2.2}R^{2.3}, CH₂—NR^{2.2}R^{2.3}, C₃₋₁₀-cycloalkyl, het, C₁₋₆-alkyl, C₁₋₃-fluoroalkyl, CF₃, CHF₂, CH₂F, C₆₋₁₀-aryl-C₁₋₆-alkylene, het-C₁₋₆-alkylene, hetaryl-C₁₋₆-alkylene, C₆₋₁₀-aryl, SO₂—CH₃, SO₂—CH₂CH₃ and SO₂—NR^{2.2}R^{2.3}, which may in turn optionally be substituted by one or more groups selected from among OH, OR^{2.1}, CF₃, CHF₂, CH₂F, oxo, halogen, CF₃, CHF₂, CH₂F, C₁₋₆-alkyl, C₆₋₁₀-aryl and NR^{2.2}R^{2.3}, or
R² denotes a group selected from among het and hetaryl, which may optionally be substituted by one or more groups selected from among halogen, OH, oxo, CF₃, CHF₂ and CH₂F or by one or more groups selected from among OR^{2.1}, C₁₋₃-alkylene-OR^{2.1}, SR^{2.1}, SO—R^{2.1}, SO₂—R^{2.1}, COOR^{2.1}, COR^{2.1}, C₁₋₆-alkanol, mono- or bicyclic C₃₋₁₀-cycloalkyl, C₆₋₁₀-aryl, C₁₋₆-alkyl, C₆₋₁₀-aryl-C₁₋₆-alkylene, hetaryl-C₁₋₆-alkylene, het, hetaryl, C₁₋₃-alkylene-OR^{2.1} and NR^{2.2}R^{2.3}, which may in turn optionally be substituted by one or more groups selected from among OH, OR^{2.1}, oxo, halogen, CF₃, CHF₂, CH₂F, C₁₋₆-alkyl, C₆₋₁₀-aryl and NR^{2.2}R^{2.3}, or wherein
NR¹R² together denotes a heterocyclic C₄₋₇ ring, which may optionally be bridged, which contains 1, 2 or 3 heteroatoms selected from among N, O and S and which may optionally be substituted by one or more groups selected from among OH, OR^{2.1}, C₁₋₃-alkylene-O^{R.1}, oxo, halogen, C₁₋₆-alkyl, C₆₋₁₀-aryl, COOR^{2.1}, CH₂—NR^{2.2}—COO—R^{2.1}, CH₂—NR^{2.2}—CO—R^{2.1}, CH₂—NR^{2.2}—CO—CH₂—NR^{2.2}R^{2.3}, CH₂—NR^{2.2}—SO₂—C₁₋₃-alkyl, CH₂—NR^{2.2}—SO₂—NR^{2.2}R^{2.3}, CH₂—NR^{2.2}—CO—NR^{2.2}R^{2.3}, CO—NR^{2.2}R^{2.3}, CH₂—NR^{2.2}R^{2.3} and NR^{2.2}R^{2.3}, and wherein
R³ is a C₆₋₁₀-aryl, which may optionally be substituted by in the ortho, para or meta position by one, two or three groups selected independently of one another from among fluorine, chlorine, bromine, hydroxy, CN, C₁₋₆-alkyl, C₁₋₃-fluoroalkyl, —C₁₋₃-alkylene-OR^{2.1}, —C₁₋₃-alkylene-NR^{2.2}R^{2.3}, —NR^{2.2}R^{2.3}, O—R^{2.1}; SO—R^{2.1}, SO₂—R^{2.1}, COOR^{2.1}, —CO—NH—(C₁₋₆-alkylene)-hetaryl, —CO—NH-hetaryl, —CO—N(CH₃)-het, —CO—N(CH₃)—(C₁₋₃-alkylene)-het, —CO—N(CH₃)—(C₁₋₃-alkylene)-hetaryl, —CO—N(C₃₋₇-cycloalkyl)-het, —CO—NR^{2.2}R^{2.3}, —CO—NH—(C₁₋₆-alkylene)-het, NR^{2.2}—CO—R^{2.1}, C₆₋₁₀-aryl, C₆₋₁₀-aryl-C₁₋₂-alkylene, het-C₁₋₂-alkylene, -het, —CO-het, CO—N(CH₃)—C₃₋₇-cycloalkyl, C₃₋₇-cycloalkyl, C₃₋₇-cycloalkyl-C₁₋₂-alkylene, hetaryl-C₁₋₂-alkylene and hetaryl, while this groups may optionally be substituted by one or more groups selected from among OH, halogen, —C₁₋₃-fluoroalkyl, oxo, methyl and phenyl, or wherein
R³ is a group selected from among het and hetaryl, which may optionally be substituted by one or more groups selected from among halogen, C₁₋₃-fluoroalkyl, CN, OH, oxo, —C₁₋₆-alkyl, —C₁₋₃-alkylene-NR^{2.2}R^{2.3}, —NR^{2.2}R^{2.3}, SO—R^{2.1}, SO₂—R^{2.1}, —O—R^{2.1}, COOR^{2.1}, SO₂—(CH₃), SO₂—(CH₂—CH₃), C₆₋₁₀-aryl, het, C₃₋₇-cycloalkyl and hetaryl, which may in turn optionally be substituted by one or more groups selected from among OH, halogen, —C₁₋₃-fluoroalkyl, C₁₋₆-alkyl, C₆₋₁₀-aryl, —COO(C₁₋₃-alkyl) and O—(C₁₋₃-alkyl), or wherein
R³ denotes —O—R^{3.1}, wherein R^{3.1} is a group selected from among —C₁₋₆-alkyl, —C₆₋₁₀-aryl, —C₁₋₃-alkylene-C₆₋₁₀-aryl, hetaryl and het, which may optionally be substituted in the ortho, para or meta position by one, two or three groups selected independently of one another from among fluorine, chlorine, bromine, hydroxy, CN, C₁₋₆-alkyl, C₁₋₃-fluoroalkyl, CO—(C₁₋₅-alkyl), —CO—(C₁₋₃-fluoroalkyl), —CO—NH—(C₁₋₆-alkylene)-hetaryl, —CO—N(C₁₋₃-alkyl)-(C₁₋₆-alkylene)-hetaryl, —CO—N(C₁₋₃-alkyl)-het, —CO—N(C₃₋₇-cycloalkyl)-het, —C₁₋₃-alkylene-OR^{2.1}, —C₁₋₃-alkylene-NR^{2.2}R^{2.3}, —NR^{2.2}R^{2.3}, O—R^{2.1}; SO—R^{2.1}, SO₂—R^{2.1}, COOH, COO—(C₁₋₄-alkyl), —O—C₁₋₃-alkylene-N(C₁₋₃-alkyl)₂, CO—NR^{2.2}R^{2.3}, NR^{2.2}—CO—R^{2.1}, C₆₋₁₀-aryl, C₆₋₁₀-aryl-C₁₋₂-alkylene, het-C₁₋₂-alkylene, —CO-het, het, —CO—C₃₋₇-cycloalkyl, —CO—N(C₁₋₃-alkyl)-C₃₋₇-cycloalkyl, C₃₋₇-cycloalkyl, C₃₋₇-cycloalkyl-C₁₋₂-alkylene, hetaryl-C₁₋₂-alkylene and hetaryl, which may in turn optionally be substituted by 1, 2, 3 or 4 groups selected independently of one another from among F, Cl, Br, methyl, O-methyl, ethyl, O-ethyl, OH, oxo and CF₃, and wherein
R⁴ denotes H, CN, OH, CF₃, CHF₂, CH₂F, F, methyl, ethyl, —O—(C₁₋₃-alkyl), —C₁₋₃-alkylene-OH, —COO(C₁₋₃-alkyl), —CO-het, —(C₁₋₂-alkylene)-NH—SO₂—(C₁₋₂-alkyl), —(C₁₋₂-alkylene)-N(C₁₋₃-alkyl)-SO₂—(C₁₋₂-alkyl), —(C₁₋₂-alkylene)-O—(C₁₋₂-alkylene)-C₆₋₁₀-aryl, —C₁₋₃-alkylene-O—C₁₋₃-alkyl, —(C₁₋₂-alkylene)-N(C₁₋₃-alkyl)-CO—(C₁₋₂-alkyl), —NH—CO—(C₁₋₃-alkylene)-O—(C₁₋₃-alkyl), —C₁₋₃-alkylene-NH—CO—(C₁₋₃-alkyl), —C₁₋₃-alkylene-NH—CO—(C₁₋₃-alkylene)-N(C₁₋₃-alkyl)₂, —O—(C₁₋₂-alkylene)-(C₆₋₁₀-aryl), —C₁₋₃-alkylene-NH—CO—(C₁₋₃-alkylene)-O—(C₁₋₃-alkyl), —CO—(C₆₋₁₀-aryl), —(C₁₋₂-alkylene)-N(C₁₋₃-alkyl)-CO—(C₁₋₂-alkylene)-O—(C₁₋₃-alkyl), wherein the aryl in the above groups may in turn optionally be substituted by one or more other groups selected from among F, Cl, Br, methyl, ethyl, propyl, isopropyl, cyclopropyl, —O-methyl, —O-ethyl, —O-propyl, —O-isopropyl, —O-cyclopropyl, —OH and CF₃ or wherein
R³ and R⁴ together form a mono- or bicyclic, unsaturated, saturated or partially saturated heterocycle, which contains 1, 2 or 3 heteroatoms selected from among N, O and S contains and which may optionally be substituted by one or more groups selected from among halogen, OH, oxo, C₁₋₃-fluoroalkyl, CN, C₁₋₆-alkyl, —O—R^{2.1}, —COOR^{2.1}, SO—R^{2.1}, SO₂—R^{2.1}, —C₁₋₃-alkylene-NR^{2.2}R^{2.3}, —NR^{2.2}R^{2.3}, C₆₋₁₀-aryl, C₃₋₇-cycloalkyl, het and hetaryl,
   as well as pharmacologically acceptable salts, diastereomers, enantiomers, racemates, hydrates or solvates thereof.

Further the instant invention relates to the aforementioned compounds of formula for their use for the treatment of diabetes mellitus or for the treatment of a microvascular or macrovascular complication of diabetes mellitus, wherein
R¹ denotes H
R² is H or C₁₋₆-alkyl, which may optionally be substituted by one or more groups selected from F, Cl, CF₃, CHF₂ or CH₂F or which may optionally be substituted by one or more groups selected from among OR^{2.1}, COOR^{2.1}, CONR^{2.2}R^{2.3}, SR^{2.1}, SO—R^{2.1}, SO₂—R^{2.1}, phenyl, het, hetaryl, a monocyclic C₃₋₇-cycloalkyl, CH₂—NR^{2.2}R^{2.3} and NR^{2.2}R^{2.3}, which in turn may optionally be substituted by one or more groups selected from among OH, F, Cl, Br, CF₃, CHF₂, CH₂F, OR^{2.1}, oxo, methyl, ethyl, propyl, isopropyl, methanol, ethanol, phenyl, COOR^{2.1}, CH₂—NR^{2.2}R^{2.3} and NR^{2.2}R^{2.3},
   wherein
het is a three- to seven-membered, monocyclic, saturated or partly saturated heterocyclic group which contains 1, 2 or 3 heteroatoms selected independently of one another from among N, S or O, and wherein
hetaryl is a five- to six-membered, monocyclic, aromatic heteroaryl which contains 1, 2 or 3 heteroatoms selected independently of one another from among N, S or O, and wherein
cycloalkyl may be saturated or partly saturated,
wherein R^{2.1} is H or a group selected from among methyl, ethyl, propyl, isopropyl, methanol, ethanol, monocyclic C₃₋₇ cycloalkyl, phenyl-C₁₋₂-alkylene, -hetaryl-C₁₋₂-alkylene,
   -het-C₁₋₂-alkylene, C₃₋₇-cycloalkyl-C₁₋₂-alkylene, phenyl, hetaryl and a het,
   which may optionally be substituted by one or more groups selected from among OH, F, Cl, methyl, ethyl, propyl, isopropyl, O-methyl, O-ethyl, O-propyl, O-isopropyl and phenyl,
wherein R^{2.2} and R^{2.3} independently of one another denote H or a group selected from among methyl, ethyl, propyl, isopropyl, monocyclic C₃₋₇-cycloalkyl, phenyl-C₁₋₃-alkylene, hetaryl-C₁₋₃-alkylene, phenyl, -het, -hetaryl, CO—NH₂, CO—NHCH₃, CON(CH₃)₂, SO₂—(C₁₋₂-alkyl), CO—R^{2.1} and COOR^{2.1}, which may optionally be substituted by one or more groups selected from among OH, F, Cl, methyl, ethyl, propyl, isopropyl, phenyl and COOR^{2.1}, or
R² denotes a monocyclic C₃₋₇ cycloalkyl, which may optionally be substituted by a group selected from among C₁₋₂-alkanol, C₁₋₃-fluoroalkyl, C₁₋₃-alkylene-OR^{2.1}, OR^{2.1}, COOR^{2.1}, SO₂—NR^{2.2}R^{2.3}, -het, —NH—CO—O-(phenyl), methyl, ethyl, propyl, isopropyl, phenyl, phenyl-C₁₋₂-alkylene, -hetaryl-C₁₋₂-alkylene, monocyclic C₃₋₇ cycloalkyl and NR^{2.2}R^{2.3}, which may optionally be substituted by one or more groups selected from among OH, OR^{2.1}, oxo, F, Cl, CF₃, CHF₂, CH₂F, methyl, ethyl, propyl, isopropyl, phenyl and NR^{2.2}R^{2.3}, or
R² denotes a phenyl which may optionally be substituted by OH, SH, F, Cl or Br or by one or more groups selected from among OR^{2.1}, COOR^{2.1}, NR^{2.2}R^{2.3}, CH₂—NR^{2.2}R^{2.3}, monocyclic C₃₋₇-cycloalkyl, -het, methyl, ethyl, propyl, isopropyl, CF₃, CHF₂, CH₂F, phenyl-C₁₋₂-alkylene, het-C₁₋₂-alkylene, hetaryl-C₁₋₂-alkylene, phenyl, SO₂—CH₃, SO₂—CH₂CH₃ and SO₂—NR^{2.2}R^{2.3}, which in turn may optionally be substituted by one or more groups selected from among OH, OR^{2.1}, oxo, F, Cl, CF₃, CHF₂, CH₂F, methyl, ethyl, propyl, isopropyl, phenyl and NR^{2.2}R^{2.3}, or
R² denotes a group selected from among het and hetaryl, which may optionally be substituted by one or more groups selected from among F, Cl, OH, oxo, CF₃, CHF₂ and CH₂F or by one or more groups selected from among OR^{2.1}, C₁₋₃-alkylene-OR^{2.1}, SR^{2.1}, SO—R^{2.1}, SO₂—R^{2.1}, COOR^{2.1}, COR^{2.1}, methanol, ethanol, monocyclic C₃₋₇-cycloalkyl, phenyl, methyl, ethyl, propyl, isopropyl, phenyl-C₁₋₂-alkylene, hetaryl-C₁₋₂-alkylene, -het, -hetaryl and NR^{2.2}R^{2.3}, which in turn may optionally be substituted by one or more groups selected from among OH, OR^{2.1}, oxo, F, Cl, CF₃, CHF₂, CH₂F, methyl, ethyl, propyl, isopropyl, butyl, isobutyl, phenyl and NR^{2.2}R^{2.3}, and wherein

R³ is a naphthalene or phenyl, which may optionally be substituted in the ortho, para or meta position by one or two groups selected independently from among fluorine, chlorine, bromine, hydroxy, CN, methyl, ethyl, propyl, isopropyl, cyclopropyl, CF₃, CHF₂, CH₂F, —OCH₃, OCH₂CH₃; SO₂—CH₃, SO—CH₃, COOCH₃, COOCH₂CH₃, —CO—NH-(methylene)-hetaryl, —CO—NH-(ethylene)-hetaryl, —CO—NH-hetaryl, —CO—N(CH₃)-het, —CO—N(CH₃)-(methylene)-het, —CO—N(CH₃)-(ethylene)-het, —CO—N(CH₃)-(methylene)-hetaryl, —CO—N(CH₃)— (ethylene)-hetaryl, —CO—N(cyclopropyl)-het, CO—NH₂, CONH(CH₃), CON(CH₃)₂, —CO—NH-(methylene)-het, —CO—NH-(ethylene)-het, —NH—CO-methyl, NCH₃—CO-methyl, —NH—CO-ethyl, NCH₃—CO-ethyl, —NH—CO-propyl, NCH₃—CO-propyl, —NH—CO-isopropyl, NCH₃—CO-isopropyl, phenyl, phenyl-methylene, phenyl-ethylene, het-methylene, het-ethylene, -het, —CO-het, —CO—N(CH₃)-het, CO—N(CH₃)-cyclopropyl, C₃₋₇-cycloalkyl, C₃₋₇-cycloalkyl-methylene, C₃₋₇-cycloalkyl-ethylene, hetaryl-methylene, hetaryl-ethylene, -hetaryl, CH₂—NH₂, CH₂—NH(CH₃), CH₂—N(CH₃)₂, —NH₂, —NH(CH₃) and —N(CH₃)₂, wherein this group may optionally be substituted by one or more groups selected from among OH, F, Cl, —CF₃, CHF₂, CH₂F, oxo, methyl and phenyl or wherein
R³ denotes a group selected from among a het and hetaryl, which may optionally be substituted by one or more groups selected from among F, Cl, Br, CF₃, CHF₂, CH₂F, CN, OH, oxo, methyl, ethyl, propyl, isopropyl, butyl, isobutyl, cyclopropyl, —O-methyl, —O-ethyl, —O-propyl, —O-isopropyl, —COO-methyl, —COO-ethyl, —COO-propyl, —COO-isopropyl, SO—(CH₃), SO—(CH₂—CH₃), SO₂—(CH₃), SO₂—(CH₂—CH₃), phenyl, CH₂—NH₂, CH₂—NH(CH₃), CH₂—N(CH₃)₂, —NH₂, —NH(CH₃), —N(CH₃)₂, het and hetaryl, which in turn may optionally be substituted by one or more groups selected from among OH, F, Cl, CF₃, CHF₂, CH₂F, methyl, ethyl, propyl, isopropyl, phenyl, —COO-methyl, —COO-ethyl and O-methyl, O-ethyl, or wherein
R³ denotes —O—R^{3.1}, wherein R^{3.1} is a group selected from among —C₁₋₃-alkyl, phenyl, —C₁₋₃-alkylene-phenyl, hetaryl and het, which is optionally substituted in the ortho, para or meta position by one, two or three groups selected independently of one another from among fluorine, chlorine, bromine, hydroxy, CN, methyl, ethyl, propyl, isopropyl, butyl, isobutyl, CF₃, CHF₂, CH₂F, CO-(methyl), CO-(ethyl), CO-(propyl), CO-(isopropyl), —CO—(CF₃), —CO—NH-(methylene)-hetaryl, —CO—NH-(ethylene)-hetaryl, —CO—N(CH₃)-(methylene)-hetaryl, —CO—N(CH₃)-(ethylene)-hetaryl, —CO—N(CH₃)-(propylene)-hetaryl, —CO—N(CH₃)-(isopropylene)-hetaryl-CO—N(CH₃)-het, —CO—N(cyclopropyl)-het, —CO—N(C₅₋₇-cycloalkyl)-het, -methylene-O-methyl, -ethylene-O-methyl, -propylene-O-methyl, -methylene-O-ethyl, -ethylene-O-ethyl, -propylene-O-ethyl, -methylene-NH₂, -methylene-NHCH₃, -methylene-N(CH₃)₂, -ethylene-NH₂, -ethylene-NHCH₃, -ethylene-N(CH₃)₂, NH₂, N(CH₃)₂, NHCH₃, —O-methyl, O-ethyl, O-propyl, O-isopropyl, O-butyl, O-isobutyl, —SO—CH₃, SO-ethyl, —SO-propyl, —SO-isopropyl, SO₂-methyl, —SO₂-ethyl, SO₂-propyl, SO₂-isopropyl, COOH, COO-(methyl), COO-(ethyl), COO-(propyl), COO-(isopropyl), —O-methylene-N(methyl)₂, —O-ethylene-N(methyl)₂, —O-methylene-N(ethyl)₂, —O-ethylene-N(ethyl)₂, CO—NH₂, CO—NH(CH₃), CO—N(CH₃)₂, —NH—CO-methyl, —NCH₃—CO-methyl, —NH—CO-ethyl, NCH₃—CO-ethyl, phenyl, phenyl-methylene, phenyl-ethylene, het-methylene, het-ethylene, —CO-het, het, —CO—C₅₋₇-cycloalkyl, —CO-cyclopropyl, —CO—N(CH₃)—C₅₋₇-cycloalkyl, CO—N(CH₃)— cyclopropyl, C₅₋₇-cycloalkyl, cyclopropyl, C₅₋₇-cycloalkyl-methylene, C₅₋₇-cycloalkyl-ethylene, cyclopropyl-methylene, cyclopropyl-ethylene, hetaryl-methylene, hetaryl-ethylene and hetaryl, which in turn may optionally be substituted by 1, 2, 3 or 4 groups selected independently of one another from among F, Cl, Br, methyl, O-methyl, ethyl, O-ethyl, OH, oxo and CF₃, and wherein
R⁴ denotes H, CN, OH, CF₃, CHF₂, CH₂F, F, methyl, ethyl, O-methyl, O-ethyl, -methylene-OH, -ethylene-OH, -propylene-OH, isopropylene-OH, —COO(methyl), —COO(ethyl), —COO(propyl), —COO(isopropyl), —CO-het, -(methylene)-NH—SO₂-(methyl), -(methylene)-NH—SO₂-(ethyl), -(ethylene)-NH—SO₂-(methyl), -(ethylene)-NH—SO₂-(ethyl), -(methylene)-N(CH₃)—SO₂-(methyl), -(methylene)-N(CH₃)—SO₂-(ethyl), -(ethylene)-N(CH₃)—SO₂-(methyl), -(ethylene)-N(CH₃)—SO₂-(ethyl), -(methylene)-O-(methylene)-phenyl, -(methylene)-O-(ethylene)-phenyl, -(ethylene)-O-(methylene)-phenyl, -(ethylene)-O-(ethylene)-phenyl, -methylene-O-methyl, -methylene-O-ethyl, -ethylene-O-methyl, -ethylene-O-ethyl, -(methylene)-N(CH₃)—CO-(methyl), -(methylene)-N(CH₃)—CO-(ethyl), -(ethylene)-N(CH₃)—CO-(methyl), -(ethylene)-N(CH₃)—CO-(ethyl), —NH—CO-(methylene)-O-(methyl), —NH—CO-(methylene)-O-(ethyl), —NH—CO-(ethylene)-O-(methyl), —NH—CO-(ethylene)-O-(ethyl), -methylene-NH—CO-(methyl), -methylene-NH—CO-(ethyl), -ethylene-NH—CO-(methyl), -ethylene-NH—CO-(ethyl), -methylene-NH—CO-(methylene)-N(methyl)₂, -methylene-NH—CO-(ethylene)-N(methyl)₂, -ethylene-NH—CO-(methylene)-N(methyl)₂, -ethylene-NH—CO-(ethylene)-N(methyl)₂, -methylene-NH—CO-(methylene)-O-(methyl), -methylene-NH—CO-(ethylene)-O-(methyl), -ethylene-NH—CO-(methylene)-O-(methyl), -methylene-NH—CO-(methylene)-O-(ethyl), -methylene-NH—CO-(ethylene)-O-(ethyl), -ethylene-NH—CO-(methylene)-O-(ethyl), -(methylene)-N(CH₃)—CO-(methylene)-O-(methyl), -(methylene)-N(CH₃)—CO-(ethylene)-O-(methyl), -(ethylene)-N(CH₃)—CO-(methylene)-O-(methyl), -(methylene)-N(CH₃)—CO-(methylene)-O-(ethyl), -(methylene-N(CH₃)—CO-(ethylene)-O-(ethyl), -(ethylene)-N(CH₃)—CO-(methylene)-O-(ethyl), —O-(methylene)-phenyl, —O-(ethylene)-phenyl, —CO-phenyl, wherein the phenyl in the above groups may optionally be substituted by one or more other groups selected from among F, Cl, Br, methyl, ethyl, propyl, —O-methyl, —O-ethyl, —O-propyl, —OH and CF₃ or wherein
R³ and R⁴ together form a mono- or bicyclic, unsaturated, saturated or partly saturated heterocyclic group which contains 1, 2 or 3 heteroatoms selected from among N, O and S and which may optionally be substituted by one or more groups selected from among F, Cl, Br, OH, oxo, CF₃, CHF₂, CH₂F, CN, methyl, ethyl, propyl, isopropyl, cyclopropyl, COO-methyl, —COO-ethyl, O-methyl, O-ethyl, SO₂—(CH₃), SO₂—(CH₂CH₃), SO—(CH₃), SO—(CH₂CH₃), CH₂—NH₂, CH₂—NH(CH₃), CH₂—N(CH₃)₂, —NH₂, —NH(CH₃), —N(CH₃)₂, phenyl, C₅₋₇-cycloalkyl, het and hetaryl,
   as well as pharmacologically acceptable salts, diastereomers, enantiomers, racemates, hydrates or solvates thereof.

Additionally the invention refers to the above mentioned compounds of formula I for their use for the treatment of diabetes mellitus or for the treatment of a microvascular or macrovascular complication of diabetes mellitus, wherein
R² is a group according to formula 3

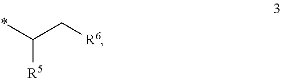

wherein R⁶ is OH or NH₂ and
wherein R⁵ is methyl, ethyl, propyl, isopropyl,
as well as pharmacologically acceptable salts, diastereomers, enantiomers, racemates, hydrates or solvates thereof.

Further, the invention relates to the aforementioned compounds of formula I for their use for the treatment of diabetes mellitus or for the treatment of a microvascular or macrovascular complication of diabetes mellitus, wherein
R² is a cyclopropyl or cyclobutyl which may optionally be substituted by another group selected from among OH, —CH₂—OH, —NH₂, CH₂—NH₂, —NH(CH₃), —N(CH₃)₂, methyl, ethyl, propyl, isopropyl, —NH—CO-(tert-butyl), —NH—CO—O-(tert-butyl), —N(CH₃)—CO-(tert-butyl), —N(CH₃)—CO—O-(tert-butyl), —CF₃, —CHF₂, CH₂F, F, Cl and Br
   as well as pharmacologically acceptable salts, diastereomers, enantiomers, racemates, hydrates or solvates thereof.

The invention also refers to the aforementioned compounds of formula I for their use for the treatment of diabetes mellitus or for the treatment of a microvascular or macrovascular complication of diabetes mellitus, wherein
R² is a phenyl which may optionally be substituted in one or both meta positions by one or more groups selected from among methyl, ethyl, propyl, isopropyl, cyclopropyl, F, Cl, Br, OH, OR^{2.1}, COOR^{2.1}, CF₃, CHF₂, CH₂F, NH₂, NH(CH₃) and N(CH₃)₂, wherein R^{2.1} may be H, methyl or ethyl,
   as well as pharmacologically acceptable salts, diastereomers, enantiomers, racemates, hydrates or solvates thereof.

In a further embodiment the invention relates to the above-mentioned compounds of formula I for their use for the treatment of diabetes mellitus or for the treatment of a microvascular or macrovascular complication of diabetes mellitus, wherein
R² denotes a group selected from among piperidine or tetrahydropyran which may optionally be substituted by one or more groups selected from among F, Cl, Br, OH, CF₃, CHF₂, CH₂F, NH₂, NHCH₃, N(CH₃)₂, oxo, methyl and methoxy,
   as well as pharmacologically acceptable salts, diastereomers, enantiomers, racemates, hydrates or solvates thereof.

In a particularly preferred embodiment the instant invention refers to one or more of the above-mentioned compounds of formula I for their use for the treatment of diabetes mellitus or for the treatment of a microvascular or macrovascular complication of diabetes mellitus, wherein the one or more compounds of formula I is/are selected from the group consisting of
1.1 (R)-2-{2-[4-(4-chlorophenyl)-piperidin-1-yl]-5-oxo-6,7-dihydro-5H-5λ⁴-thieno[3,2-d]pyrimidin-4-ylamino}-3-methylbutan-1-ol
1.2 (1-{2-[4-(4-chlorophenyl)-piperidin-1-yl]-5-oxo-6,7-dihydro-5H-5λ⁴-thieno[3,2-d]pyrimidin-4-ylamino}-cyclopropyl)-methanol
1.3 (R)-2-{2-[4-(4-chlorophenyl)-piperidin-1-yl]-5-oxo-6,7-dihydro-5H-5λ⁴-thieno[3,2-d]pyrimidin-4-ylamino}-pentan-1-ol
1.4 (R)-1-{2-[4-(4-chlorophenyl)-piperidin-1-yl]-5-oxo-6,7-dihydro-5H-5λ⁴-thieno[3,2-d]pyrimidin-4-ylamino}-1-(4-fluorophenyl)-2-methylpropan-2-ol
1.5 (S)-5-{2-[4-(4-chlorophenyl)-piperidin-1-yl]-5-oxo-6,7-dihydro-5H-5λ⁴-thieno[3,2-d]pyrimidin-4-ylamino}-1-methylpiperidin-2-one
1.6 {2-[4-(4-chlorophenyl)-piperidin-1-yl]-5-oxo-6,7-dihydro-5H-5λ⁴-thieno[3,2-d]pyrimidin-4-yl}-(tetrahydropyran-4-yl)-amine
1.7 1-(4-(1-hydroxymethylcyclopropylamino)-5-oxo-6,7-dihydro-5H-5λ⁴-thieno[3,2-d]pyrimidin-2-yl)-3′-methyl-1′H-spiro[piperidin-4,4′-quinazolin]-2′(3′H)-one
1.8 {1-[2-(4-benzo[d]isoxazol-3-yl-piperidin-1-yl)-5-oxo-6,7-dihydro-5H-5λ⁴-thieno[3,2-d]pyrimidin-4-ylamino]-cyclopropyl}-methanol
1.9 (1-{2-[4-(2-ethyl-5-fluoro-1H-indol-3-yl)-piperidin-1-yl]-5-oxo-6,7-dihydro-5H-5λ⁴-thieno[3,2-d]pyrimidin-4-ylamino}-cyclopropyl)-methanol
1.10 1-[4-((S)-1-methyl-6-oxopiperidin-3-ylamino)-5-oxo-6,7-dihydro-5H-5λ⁴-thieno[3,2-d]pyrimidin-2-yl]-4-phenylpiperidin-4-carbonitrile
1.11 3′-methyl-1-(4-(tetrahydro-2H-pyran-4-ylamino)-5-oxo-6,7-dihydro-5H-5λ⁴-thieno[3,2-d]pyrimidin-2-yl)-1′H-spiro[piperidin-4,4′-quinazolin]-2′(3′H)-one
1.12 (3-fluorophenyl)-[5-oxo-2-(3,4,5,6-tetrahydro-2H-[4,4′]bipyridinyl-1-yl)-6,7-dihydro-5H-5λ⁴-thieno[3,2-d]pyrimidin-4-yl]-amine
1.13 {2-[4-(2-ethyl-5-fluoro-1H-indol-3-yl)-piperidin-1-yl]-5-oxo-6,7-dihydro-5H-5λ⁴-thieno[3,2-d]pyrimidin-4-yl}-(3-fluorophenyl)-amine
1.14 (1-{2-[4-(2,4-difluorophenyl)-piperidin-1-yl]-5-oxo-6,7-dihydro-5H-5λ⁴-thieno[3,2-d]pyrimidin-4-ylamino}-cyclopropyl)-methanol
1.15 {2-[4-(2,4-difluorophenyl)-piperidin-1-yl]-5-oxo-6,7-dihydro-5H-5λ⁴-thieno[3,2-d]pyrimidin-4-yl}-(tetrahydropyran-4-yl)-amine
1.16 (S)-5-[2-(4-benzoxazol-2-yl-piperidin-1-yl)-5-oxo-6,7-dihydro-5H-5λ⁴-thieno[3,2-d]pyrimidin-4-ylamino]-1-methylpiperidin-2-one
1.17 (1-{2-[4-(6-fluorobenzo[d]isoxazol-3-yl)-piperidin-1-yl]-5-oxo-6,7-dihydro-5H-5λ⁴-thieno[3,2-d]pyrimidin-4-ylamino}-cyclopropyl)-methanol
1.18 (1-{2-[4-(5-fluorobenzo[d]isoxazol-3-yl)-piperidin-1-yl]-5-oxo-6,7-dihydro-5H-5λ⁴-thieno[3,2-d]pyrimidin-4-ylamino}-cyclopropyl)-methanol
1.19 {2-[4-(5-furan-2-yl-2H-pyrazol-3-yl)-piperidin-1-yl]-5-oxo-6,7-dihydro-5H-5λ⁴-thieno[3,2-d]pyrimidin-4-yl}-(tetrahydropyran-4-yl)-amine
1.20 (3-fluorophenyl)-{5-oxo-2-[4-(3-pyridin-4-yl-[1,2,4]oxadiazol-5-yl)-piperidin-1-yl]-6,7-dihydro-5H-5λ⁴-thieno[3,2-d]pyrimidin-4-yl}-amine
1.21 (R)-3-methyl-2-{5-oxo-2-[4-(3-pyridin-4-yl-[1,2,4]oxadiazol-5-yl)-piperidin-1-yl]-6,7-dihydro-5H-5λ⁴-thieno[3,2-d]pyrimidin-4-ylamino}-butan-1-ol
1.22 (S)-5-{2-[4-(4-fluorophenoxy)-piperidin-1-yl]-5-oxo-6,7-dihydro-5H-5λ4-thieno[3,2-d]pyrimidin-4-ylamino}-1-methylpiperidin-2-one
1.23 (2-{4-[4-(4,5-dihydrooxazol-2-yl)-phenoxy]-piperidin-1-yl}-5-oxo-6,7-dihydro-5H-5λ⁴-thieno[3,2-d]pyrimidin-4-yl)-(tetrahydropyran-4-yl)-amine
1.24 4-{1-[5-oxo-4-(tetrahydropyran-4-ylamino)-6,7-dihydro-5H-5λ⁴-thieno[3,2-d]pyrimidin-2-yl]-piperidin-4-yloxy}-benzoic acid
1.25 2-(1-{2-[4-(4-chlorophenyl)-piperidin-1-yl]-5-oxo-6,7-dihydro-5H-5λ⁴-thieno[3,2-d]pyrimidin-4-ylamino}-cyclopropyl)-propan-2-ol
1.26 {2-[4-(5-tert-butyl-1-methyl-1H-indol-3-yl)-piperidin-1-yl]-5-oxo-6,7-dihydro-5H-5λ⁴-thieno[3,2-d]pyrimidin-4-yl}-(tetrahydropyran-4-yl)-amine
1.27 2-[4-(5-furan-2-yl-1-methyl-1H-pyrazol-3-yl)-piperidin-1-yl]-5-oxo-6,7-dihydro-5H-5λ⁴-thieno[3,2-d]pyrimidin-4-yl}-(tetrahydropyran-4-yl)-amine
1.28 (S)-5-(2-{4-[4-(4,5-dihydrooxazol-2-yl)-phenoxy]-piperidin-1-yl}-5-oxo-6,7-dihydro-5H-5λ⁴-thieno[3,2-d]pyrimidin-4-ylamino)-1-methylpiperidin-2-one
1.29 {2-[4-(5-furan-2-yl-2-methyl-2H-pyrazol-3-yl)-piperidin-1-yl]-5-oxo-6,7-dihydro-5H-5λ⁴-thieno[3,2-d]pyrimidin-4-yl}-(tetrahydropyran-4-yl)-amine
1.30 {2-[4-(1-methyl-1H-imidazo[4,5-c]pyridin-2-yl)-piperidin-1-yl]-5-oxo-6,7-dihydro-5H-5λ⁴-thieno[3,2-d]pyrimidin-4-yl}-(tetrahydropyran-4-yl)-amine
1.31 2-methoxy-N-{1-[5-oxo-4-(tetrahydropyran-4-ylamino)-6,7-dihydro-5H-5λ⁴-thieno[3,2-d]pyrimidin-2-yl]-4-phenylpiperidin-4-ylmethyl}-acetamide
1.32 N-cyclopropyl-N-methyl-4-{1-[5-oxo-4-(tetrahydropyran-4-ylamino)-6,7-dihydro-5H-5λ⁴-thieno[3,2-d]pyrimidin-2-yl]-piperidin-4-yl}-benzamide
1.33 N-cyclopropyl-N-methyl-4-{1-[5-oxo-4-(tetrahydropyran-4-ylamino)-6,7-dihydro-5H-5λ⁴-thieno[3,2-d]pyrimidin-2-yl]-piperidin-4-yloxy}-benzamide
1.34 {5-oxo-2-[4-(pyridin-4-yloxy)-piperidin-1-yl]-6,7-dihydro-5H-5λ⁴-thieno[3,2-d]pyrimidin-4-yl}-(tetrahydropyran-4-yl)-amine
1.35 {2-[4-(4-chlorophenoxy)-piperidin-1-yl]-5-oxo-6,7-dihydro-5H-5λ⁴-thieno[3,2-d]pyrimidin-4-yl}-(tetrahydropyran-4-yl)-amine
1.36 (S)-1-methyl-5-{2-[4-(5-methyl-4-phenyloxazol-2-yl)-piperidin-1-yl]-5-oxo-6,7-dihydro-5H-5λ⁴-thieno[3,2-d]pyrimidin-4-ylamino}-piperidin-2-one
1.37 (1-{2-[4-(5-methyl-4-phenyloxazol-2-yl)-piperidin-1-yl]-5-oxo-6,7-dihydro-5H-5λ⁴-thieno[3,2-d]pyrimidin-4-ylamino}-cyclopropyl)-methanol
1.38 (S)-5-{2-[4-(4,5-diphenyloxazol-2-yl)-piperidin-1-yl]-5-oxo-6,7-dihydro-5H-5λ⁴-thieno[3,2-d]pyrimidin-4-ylamino}-1-methylpiperidin-2-one
1.39 {4-(4-chlorophenyl)-1-[5-oxo-4-(tetrahydropyran-4-ylamino)-6,7-dihydro-5H-5λ⁴-thieno[3,2-d]pyrimidin-2-yl]-piperidin-4-yl}-methanol
1.40 [1-(2-{4-[5-(4-chlorophenyl)-4-methyloxazol-2-yl]-piperidin-1-yl}-5-oxo-6,7-dihydro-5H-5λ⁴-thieno[3,2-d]pyrimidin-4-ylamino)-cyclopropyl]-methanol
1.41 4-(4-chlorophenyl)-1-[5-oxo-4-(tetrahydropyran-4-ylamino)-6,7-dihydro-5H-5λ⁴-thieno[3,2-d]pyrimidin-2-yl]-piperidin-4-ol
1.42 {2-[4-(4-chlorophenyl)-4-methoxypiperidin-1-yl]-5-oxo-6,7-dihydro-5H-5λ⁴-thieno[3,2-d]pyrimidin-4-yl}-(tetrahydropyran-4-yl)-amine
1.43 4-{1-[4-(1-hydroxymethylcyclopropylamino)-5-oxo-6,7-dihydro-5H-5λ⁴-thieno[3,2-d]pyrimidin-2-yl]-piperidin-4-yloxy}-benzonitrile
1.44 5-oxo-2-[4-(4,5,6,7-tetrahydrobenzoxazol-2-yl)-piperidin-1-yl]-6,7-dihydro-5H-5λ⁴-thieno[3,2-d]pyrimidin-4-yl}-(tetrahydropyran-4-yl)-amine
1.45 (S)-5-{2-[4-(4-chlorophenyl)-piperidin-1-yl]-5,5-dioxo-6,7-dihydro-5H-5λ⁶-thieno[3,2-d]pyrimidin-4-ylamino}-1-methylpiperidin-2-one
1.46 (1-{2-[4-(5-Chloro-pyrimidin-2-yl)-piperidin-1-yl]-5-oxo-6,7-dihydro-5H-5λ⁴-thieno[3,2-d]pyrimidin-4-yl-amino}-cyclobutyl)-methanol and
1.47 (1-{2-[4-(4-Chloro-phenyl)-piperidin-1-yl]-5-oxo-6,7-dihydro-5H-5λ⁴-thieno[3.2-d]pyrimidin-4-yl-amino}-cyclobutyl)-methanol.

In a particularly preferred embodiment the instant invention relates to one of the above compounds of formula I for the treatment of diabetes mellitus type 1.

In a further particularly preferred embodiment the instant invention relates to one of the above compounds of formula I for the treatment of diabetes mellitus type 2.

In a further particularly preferred embodiment the instant invention refers to the above-identified compounds of formula I for the treatment of a microvascular complication of diabetes mellitus selected from the group consisting of diabetic retinopathy, diabetic nephropathy, diabetic neuropathy, diabetic foot and diabetic ulcer.

In an additional particularly preferred embodiment the instant invention relates to the above-mentioned compounds of formula I for the treatment of a macrovascular complication of diabetes mellitus selected from the group consisting of myocardial infarct, acute coronary syndrome, unstable angina pectoris, stable angina pectoris, stroke, peripheral arterial occlusive disease, cardiomyopathy, heart failure, heart rhythm disorders and vascular restenosis.

In a further particularly preferred embodiment the instant invention relates to a pharmaceutical preparation comprising one or more of the above-identified compounds of formula I and one or more active agents selected from the group consisting of metformin, sulphonylureas, nateglinide, repaglinide, thiazolidinediones, dipeptidylpeptidase 4 inhibitors (DPP4-inhibitors), peroxisome proliferator-activated receptor gamma agonists (PPAR-gamma-agonists), alpha-glucosidase inhibitors, insulin, insulin analogues, glucagon-like-peptide 1 (GLP-1) and glucagon-like-peptide 1 analogues (GLP1-analogues).

Further the instant invention relates to the use of a compound of formula 1 for the manufacture of a medicament for the treatment of diabetes mellitus or for the treatment of a microvascular or macrovascular complication of diabetes mellitus

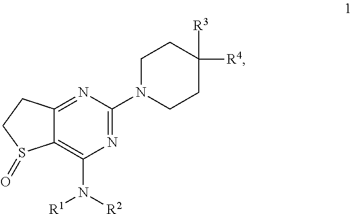

wherein
R¹ denotes H, C₁₋₆-alkyl,
R² is H or a group selected from among C₁₋₁₀-alkyl and C₂₋₆-alkenyl, which may optionally be substituted by one or more groups selected from halogen and C₁₋₃-fluoroalkyl or which may optionally be substituted by one or more groups selected from among OR^{2.1}, COOR^{2.1}, CONR^{2.2}R^{2.3}, SR^{2.1}, SO—R^{2.1}, SO₂—R^{2.1}, C₆₋₁₀-aryl, -het, hetaryl, a mono- or bicyclic —C₃₋₁₀-cycloalkyl, CH₂—NR^{2.2}R^{2.3} and NR^{2.2}R^{2.3}, which in turn may optionally be substituted by one or more groups selected from among OH, halogen, OR^{2.1}, oxo, CF₃, CHF₂, CH₂F, C₁₋₆-alkyl, C₁₋₆-alkanol, C₆₋₁₀-aryl, COOR^{2.1}, CH₂—NR^{2.2}R^{2.3} and NR^{2.2}R^{2.3},
wherein
het denotes a three- to eleven-membered, mono- or bicyclic, saturated or partially saturated, optionally anellated or optionally bridged heterocycle is, which contains 1, 2, 3 or 4 heteroatoms selected independently of one another from among N, S or O contains, and wherein
hetaryl is a five- to ten-membered, mono- or bicyclic, optionally anellated heteroaryl, which contains 1, 2, 3 or 4 heteroatoms selected independently of one another from among N, S or O, and wherein
cycloalkyl may be saturated or partially saturated,
wherein R^{2.1} is H or is a group selected from among C₁₋₆-alkyl, C₁₋₆-alkanol, C₁₋₃-haloalkyl, mono- or bicyclic, —C₃₋₁₀-cycloalkyl, C₆₋₁₀-aryl-C₁₋₆-alkylene, hetaryl-C₁₋₆-alkylene, het-C₁₋₆-alkylene, C₃₋₁₀-cycloalkyl-C₁₋₆-alkylene, a mono- or bicyclic C₆₋₁₀-aryl, heteroaryl and a -het, which may optionally be substituted by one or more groups selected from among OH, O—(C₁₋₃-alkyl), halogen, C₁₋₆-alkyl and C₆₋₁₀-aryl,
wherein R^{2.2} and R^{2.3} independently of one another denote H or a group selected from among C₁₋₆-alkyl, mono- or bicyclic C₃₋₁₀ cycloalkyl, C₆₋₁₀-aryl-C₁₋₆-alkylene, hetaryl-C₁₋₆-alkylene, mono- or bicyclic C₆₋₁₀-aryl, het, hetaryl, CO—NH₂, CO—NHCH₃, —CO—N(CH₃)₂, SO₂—(C₁-C₂-alkyl), CO—R^{2.1} and COOR^{2.1}, which may optionally be substituted by one or more groups selected from among OH, halogen, C₁₋₆-alkyl, C₆₋₁₀-aryl and COOR^{2.1}, or
R² denotes a mono- or polycyclic C₃₋₁₀ cycloalkyl, which may optionally be bridged one or more times via C₁₋₃-alkyl groups and which may optionally be substituted by a group selected from among branched or unbranched C₁₋₆-alkanol, C₁₋₃-fluoroalkyl, C₁₋₃-alkylene-OR^{2.1}, OR^{2.1}, COOR^{2.1}, —SO₂—NR^{2.2}R^{2.3}, het, —NH—CO—O—(C₁₋₆-alkyl), —NH—CO—(C₁₋₆-alkyl), —NH—CO—O—(C₆₋₁₀-aryl), —NH—CO—(C₆₋₁₀-aryl), —NH—CO—O-hetaryl, —NH—CO-hetaryl, —NH—CO—O—(C₁₋₃-alkylene)-(C₆₋₁₀-aryl), —NH—CO—(C₁₋₃-alkylene)-(C₆₋₁₀-aryl), —N(C₁₋₃-alkyl)-CO—(C₁₋₆-alkyl), —N(C₁₋₃-alkyl)-CO—O—(C₆₋₁₀-aryl), —N(C₁₋₃-alkyl)-CO—(C₆₋₁₀-aryl), —N(C₁₋₃-alkyl)-CO—O-hetaryl, —N(C₁₋₃-alkyl)-CO-hetaryl, —N(C₁₋₃-alkyl)-CO—O—(C₁₋₃-alkylene)-(C₆₋₁₀-aryl), —N(C₁₋₃-alkyl)-CO—(C₁₋₃-alkylene)-(C₆₋₁₀-aryl), C₆₋₁₀-aryl, C₁₋₆-alkyl, C₆₋₁₀-aryl-C₁₋₆-alkylene, hetaryl-C₁₋₆-alkylene, mono- or bicyclic C₃₋₁₀ cycloalkyl and NR^{2.2}R^{2.3}, which may optionally be substituted by one or more groups selected from among OH, OR^{2.1}, oxo, halogen, CF₃, CHF₂, CH₂F, C₁₋₆alkyl, C₆₋₁₀-aryl and NR^{2.2}R^{2.3}, or
R² denotes a mono- or polycyclic C₆₋₁₀-aryl, which may optionally be substituted by OH, SH or halogen or by one or more groups selected from among OR^{2.1}, COOR^{2.1}, NR^{2.2}R^{2.3}, CH₂—NR^{2.2}R^{2.3}, C₃₋₁₀-cycloalkyl, het, C₁₋₆-alkyl, C₁₋₃-fluoroalkyl, CF₃, CHF₂, CH₂F, C₆₋₁₀-aryl-C₁₋₆-alkylene, het-C₁₋₆-alkylene, hetaryl-C₁₋₆-alkylene, C₆₋₁₀-aryl, SO₂—CH₃, SO₂—CH₂CH₃ and SO₂—NR^{2.2}R^{2.3}, which may in turn optionally be substituted by one or more groups selected from among OH, OR^{2.1}, CF₃, CHF₂, CH₂F, oxo, halogen, CF₃, CHF₂, CH₂F, C₁₋₆-alkyl, C₆₋₁₀-aryl and NR^{2.2}R^{2.3}, or
R² denotes a group selected from among het and hetaryl, which may optionally be substituted by one or more groups selected from among halogen, OH, oxo, CF₃, CHF₂ and CH₂F or by one or more groups selected from among OR^{2.1}, C₁₋₃-alkylene-OR^{2.1}, SR^{2.1}, SO—R^{2.1}, SO₂—R^{2.1}, COOR^{2.1}, COR^{2.1}, C₁₋₆-alkanol, mono- or bicyclic C₃₋₁₀-cycloalkyl, C₆₋₁₀-aryl, C₁₋₆-alkyl, C₆₋₁₀-aryl-C₁₋₆-alkylene, hetaryl-C₁₋₆-alkylene, het, hetaryl, C₁₋₃-alkylene-OR^{2.1} and NR^{2.2}R^{2.3}, which may in turn optionally be substituted by one or more groups selected from among OH, OR^{2.1}, oxo, halogen, CF₃, CHF₂, CH₂F, C₁₋₆-alkyl, C₆₋₁₀-aryl and NR^{2.2}R^{2.3}, or wherein
NR¹R² together denotes a heterocyclic C₄₋₇ ring, which may optionally be bridged, which contains 1, 2 or 3 heteroatoms selected from among N, O and S and which may optionally be substituted by one or more groups selected from among OH, OR^{2.1}, C₁₋₃-alkylene-O^{R.1}, oxo, halogen, C₁₋₆-alkyl, C₆₋₁₀-aryl, COOR^{2.1}, CH₂—NR^{2.2}—COO—R^{2.1}, CH₂—NR^{2.2}—CO—R^{2.1}, CH₂—NR^{2.2}—CO—CH₂—NR^{2.2}R^{2.3}, CH₂—NR^{2.2}—SO₂—C₁₋₃-alkyl, CH₂—NR^{2.2}—SO₂—NR^{2.2}R^{2.3}, CH₂—NR^{2.2}—CO—NR^{2.2}R^{2.3}, CO—NR^{2.2}R^{2.3}, CH₂—NR^{2.2}R^{2.3} and NR^{2.2}R^{2.3}, and wherein
R³ is a C₆₋₁₀-aryl, which may optionally be substituted by in the ortho, para or meta position by one, two or three groups selected independently of one another from among fluorine, chlorine, bromine, hydroxy, CN, C₁₋₆-alkyl, C₁₋₃-fluoroalkyl, —C₁₋₃-alkylene-OR^{2.1}, —C₁₋₃-alkylene-NR^{2.2}R^{2.3}, —NR^{2.2}R^{2.3}, O—R^{2.1}; SO—R^{2.1}, SO₂—R^{2.1}, COOR^{2.1}, —CO—NH—(C₁₋₆-alkylene)-hetaryl, —CO—NH-hetaryl, —CO—N(CH₃)-het, —CO—N(CH₃)—(C₁₋₃-alkylene)-het, —CO—N(CH₃)—(C₁₋₃-alkylene)-hetaryl, —CO—N(C₃₋₇-cycloalkyl)-het, —CO—NR^{2.2}R^{2.3}, —CO—NH—(C₁₋₆-alkylene)-het, —NR^{2.2}—CO—R^{2.1}, C₆₋₁₀-aryl, —C₆₋₁₀-aryl-C₁₋₂-alkylene, -het-C₁₋₂-alkylene, -het, —CO-het, —CO—N(CH₃)—C₃₋₇-cycloalkyl, —C₃₋₇-cycloalkyl-C₁₋₂-alkylene, -hetaryl-C₁₋₂-alkylene and -hetaryl, while this groups may optionally be substituted by one or more groups selected from among OH, halogen, —C₁₋₃-fluoroalkyl, oxo, methyl and phenyl, or wherein
R³ is a group selected from among het and hetaryl, which may optionally be substituted by one or more groups selected from among halogen, C₁₋₃-fluoroalkyl, CN, OH, oxo, —C₁₋₆-alkyl, —C₁₋₃-alkylene-NR^{2.2}R^{2.3}, —NR^{2.2}R^{2.3}, SO—R^{2.1}, SO₂—R^{2.1}, —O—R^{2.1}, —COOR^{2.1}, SO₂—(CH₃), SO₂—(CH₂—CH₃), C₆₋₁₀-aryl, het, C₃₋₇-cycloalkyl and hetaryl, which may in turn optionally be substituted by one or more groups selected from among OH, halogen, —C₁₋₃-fluoroalkyl, C₁₋₆-alkyl, C₆₋₁₀-aryl, —COO(C₁₋₃-alkyl) and O—(C₁₋₃-alkyl), or wherein
R³ denotes —O—R^{3.1}, wherein R^{3.1} is a group selected from among —C₁₋₆-alkyl, —C₆₋₁₀-aryl, —C₁₋₃-alkylene-C₆₋₁₀-aryl, hetaryl and het, which may optionally be substituted in the ortho, para or meta position by one, two or three groups selected independently of one another from among fluorine, chlorine, bromine, hydroxy, CN, C₁₋₆-alkyl, C₁₋₃-fluoroalkyl, CO—(C₁₋₅-alkyl), —CO—(C₁₋₃-fluoroalkyl), —CO—NH—(C₁₋₆-alkylene)-hetaryl, —CO—N(C₁₋₃-alkyl)-(C₁₋₆-alkylene)-hetaryl, —CO—N(C₁₋₃-alkyl)-het, —CO—N(C₃₋₇-cycloalkyl)-het, —C₁₋₃-alkylene-OR^{2.1}, —C₁₋₃-alkylene-NR^{2.2}R^{2.3}, —NR^{2.2}R^{2.3}, O—R^{2.1}; SO—R^{2.1}, SO₂—R^{2.1}, COOH, COO—(C₁₋₄-alkyl), —O—C₁₋₃-alkylene-N(C₁₋₃-alkyl)₂, CO—NR^{2.2}R^{2.3}, NR^{2.2}—CO—R^{2.1}, C₆₋₁₀-aryl, C₆₋₁₀-aryl-C₁₋₂-alkylene, het-C₁₋₂-alkylene, —CO-het, het, —CO—C₃₋₇-cycloalkyl, —CO—N(C₁₋₃-alkyl)-C₃₋₇-cycloalkyl, C₃₋₇-cycloalkyl, C₃₋₇-cycloalkyl-C₁₋₂-alkylene, hetaryl-C₁₋₂-alkylene and hetaryl, which may in turn optionally be substituted by 1, 2, 3 or 4 groups selected independently of one another from among F, Cl, Br, methyl, O-methyl, ethyl, O-ethyl, OH, oxo and CF₃, and wherein
R⁴ denotes H, CN, OH, CF₃, CHF₂, CH₂F, F, methyl, ethyl, —O—(C₁₋₃-alkyl), —C₁₋₃-alkylene-OH, —COO(C₁₋₃-alkyl), —CO-het, —(C₁₋₂-alkylene)-NH—SO₂—(C₁₋₂-alkyl), —(C₁₋₂-alkylene)-N(C₁₋₃-alkyl)-SO₂—(C₁₋₂-alkyl), —(C₁₋₂-alkylene)-O—(C₁₋₂-alkylene)-C₆₋₁₀-aryl, —C₁₋₃-alkylene-O—C₁₋₃-alkyl, —(C₁₋₂-alkylene)-N(C₁₋₃-alkyl)-CO—(C₁₋₂-alkyl), —NH—CO—(C₁₋₃-alkylene)-O—(C₁₋₃-alkyl), —C₁₋₃-alkylene-NH—CO—(C₁₋₃-alkyl), —C₁₋₃-alkylene-NH—CO—(C₁₋₃-alkylene)-N(C₁₋₃-alkyl)₂, —O—(C₁₋₂-alkylene)-(C₆₋₁₀-aryl), —C₁₋₃-alkylene-NH—CO—(C₁₋₃-alkylene)-O—(C₁₋₃-alkyl), —CO—(C₆₋₁₀-aryl), —(C₁₋₂-alkylene)-N(C₁₋₃-alkyl)-CO—(C₁₋₂-alkylene)-O—(C₁₋₃-alkyl), wherein the aryl in the above groups may in turn optionally be substituted by one or more other groups selected from among F, Cl, Br, methyl, ethyl, propyl, isopropyl, cyclopropyl, —O-methyl, —O-ethyl, —O-propyl, —O-isopropyl, —O-cyclopropyl, —OH and CF₃ or wherein
R³ and R⁴ together form a mono- or bicyclic, unsaturated, saturated or partially saturated heterocycle, which contains 1, 2 or 3 heteroatoms selected from among N, O and S contains and which may optionally be substituted by one or more groups selected from among halogen, OH, oxo, C₁₋₃-fluoroalkyl, CN, C₁₋₆-alkyl, —O—R^{2.1}, —COOR^{2.1}, SO—R^{2.1}, SO₂—R^{2.1}, —C₁₋₃-alkylene-NR^{2.2}R^{2.3}, —NR^{2.2}R^{2.3}, C₆₋₁₀-aryl, C₃₋₇-cycloalkyl, het and hetaryl,
   as well as pharmacologically acceptable salts, diastereomers, enantiomers, racemates, hydrates or solvates thereof.

Further the instant invention relates to the use of one of the above-mentioned compounds of formula I for the manufacture of a medicament for the treatment of diabetes mellitus or for the treatment of a microvascular or macrovascular complication of diabetes mellitus, wherein
R¹ denotes H
R² is H or C₁₋₆-alkyl, which may optionally be substituted by one or more groups selected from F, Cl, CF₃, CHF₂ or CH₂F or which may optionally be substituted by one or more groups selected from among OR^{2.1}, COOR^{2.1}, CONR^{2.2}R^{2.3}, SR^{2.1}, SO—R^{2.1}, SO₂—R^{2.1}, phenyl, het, hetaryl, a monocyclic C₃₋₇-cycloalkyl, CH₂—NR^{2.2}R^{2.3} and NR^{2.2}R^{2.3}, which in turn may optionally be substituted by one or more groups selected from among OH, F, Cl, Br, CF₃, CHF₂, CH₂F, OR^{2.1}, oxo, methyl, ethyl, propyl, isopropyl, methanol, ethanol, phenyl, COOR^{2.1}, CH₂—NR^{2.2}R^{2.3} and NR^{2.2}R^{2.3},
   wherein
het is a three- to seven-membered, monocyclic, saturated or partly saturated heterocyclic group which contains 1, 2 or 3 heteroatoms selected independently of one another from among N, S or O, and wherein
hetaryl is a five- to six-membered, monocyclic, aromatic heteroaryl which contains 1, 2 or 3 heteroatoms selected independently of one another from among N, S or O, and wherein
cycloalkyl may be saturated or partly saturated,
wherein R^{2.1} is H or a group selected from among methyl, ethyl, propyl, isopropyl, methanol, ethanol, monocyclic C₃₋₇ cycloalkyl, phenyl-C₁₋₂-alkylene, -hetaryl-C₁₋₂-alkylene, -het-C₁₋₂-alkylene, C₃₋₇-cycloalkyl-C₁₋₂-alkylene, phenyl, hetaryl and a het, which may optionally be substituted by one or more groups selected from among OH, F, Cl, methyl, ethyl, propyl, isopropyl, O-methyl, O-ethyl, O-propyl, O-isopropyl and phenyl,
wherein R^{2.2} and R^{2.3} independently of one another denote H or a group selected from among methyl, ethyl, propyl, isopropyl, monocyclic C₃₋₇-cycloalkyl, phenyl-C₁₋₃-alkylene, hetaryl-C₁₋₃-alkylene, phenyl, -het, -hetaryl, CO—NH₂, CO—NHCH₃, CON(CH₃)₂, SO₂—(C₁₋₂-alkyl), CO—R^{2.1} and COOR^{2.1}, which may optionally be substituted by one or more groups selected from among OH, F, Cl, methyl, ethyl, propyl, isopropyl, phenyl and COOR^{2.1}, or
R² denotes a monocyclic C₃₋₇ cycloalkyl, which may optionally be substituted by a group selected from among C₁₋₂-alkanol, C₁₋₃-fluoroalkyl, C₁₋₃-alkylene-OR^{2.1}, OR^{2.1}, COOR^{2.1}, SO₂—NR^{2.2}R^{2.3}, -het, —NH—CO—O-(phenyl), methyl, ethyl, propyl, isopropyl, phenyl, phenyl-C₁₋₂-alkylene, -hetaryl-C₁₋₂-alkylene, monocyclic C₃₋₇ cycloalkyl and NR^{2.2}R^{2.3}, which may optionally be substituted by one or more groups selected from among OH, OR^{2.1}, oxo, F, Cl, CF₃, CHF₂, CH₂F, methyl, ethyl, propyl, isopropyl, phenyl and NR^{2.2}R^{2.3}, or
R² denotes a phenyl which may optionally be substituted by OH, SH, F, Cl or Br or by one or more groups selected from among OR^{2.1}, COOR^{2.1}, NR^{2.2}R^{2.3}, CH₂—NR^{2.2}R^{2.3}, monocyclic C₃₋₇-cycloalkyl, -het, methyl, ethyl, propyl, isopropyl, CF₃, CHF₂, CH₂F, phenyl-C₁₋₂-alkylene, het-C₁₋₂-alkylene, hetaryl-C₁₋₂-alkylene, phenyl, SO₂—CH₃, SO₂—CH₂CH₃ and SO₂—NR^{2.2}R^{2.3}, which in turn may optionally be substituted by one or more groups selected from among OH, OR^{2.1}, oxo, F, Cl, CF₃, CHF₂, CH₂F, methyl, ethyl, propyl, isopropyl, phenyl and NR^{2.2}R^{2.3}, or
R² denotes a group selected from among het and hetaryl, which may optionally be substituted by one or more groups selected from among F, Cl, OH, oxo, CF₃, CHF₂ and CH₂F or by one or more groups selected from among OR^{2.1}, C₁₋₃-alkylene-OR^{2.1}, SR^{2.1}, SO—R^{2.1}, COOR^{2.1}, COR^{2.1}, methanol, ethanol, monocyclic C₃₋₇-cycloalkyl, phenyl, methyl, ethyl, propyl, isopropyl, phenyl-C₁₋₂-alkylene, hetaryl-C₁₋₂-alkylene, -het, -hetaryl and NR^{2.2}R^{2.3}, which in turn may optionally be substituted by one or more groups selected from among OH, OR^{2.1}, oxo, F, Cl, CF₃, CHF₂, CH₂F, methyl, ethyl, propyl, isopropyl, butyl, isobutyl, phenyl and NR^{2.2}R^{2.3}, and wherein
R³ is a naphthalene or phenyl, which may optionally be substituted in the ortho, para or meta position by one or two groups selected independently from among fluorine, chlorine, bromine, hydroxy, CN, methyl, ethyl, propyl, isopropyl, cyclopropyl, CF₃, CHF₂, CH₂F, —OCH₃, OCH₂CH₃; SO₂—CH₃, SO—CH₃, COOCH₃, COOCH₂CH₃, —CO—NH-(methylene)-hetaryl, —CO—NH-(ethylene)-hetaryl, —CO—NH-hetaryl, —CO—N(CH₃)-het, —CO—N(CH₃)-(methylene)-het, —CO—N(CH₃)-(ethylene)-het, —CO—N(CH₃)-(methylene)-hetaryl, —CO—N(CH₃)— (ethylene)-hetaryl, —CO—N(cyclopropyl)-het, CO—NH₂, CONH(CH₃), CON(CH₃)₂, —CO—NH-(methylene)-het, —CO—NH-(ethylene)-het, —NH—CO-methyl, NCH₃—CO-methyl, —NH—CO-ethyl, NCH₃—CO-ethyl, —NH—CO-propyl, NCH₃—CO-propyl, —NH—CO-isopropyl, NCH₃—CO-isopropyl, phenyl, phenyl-methylene, phenyl-ethylene, het-methylene, het-ethylene, -het, —CO-het, —CO—N(CH₃)-het, CO—N(CH₃)-cyclopropyl, C₃₋₇-cycloalkyl, C₃₋₇-cycloalkyl-methylene, C₃₋₇-cycloalkyl-ethylene, hetaryl-methylene, hetaryl-ethylene, -hetaryl, CH₂—NH₂, CH₂—NH(CH₃), CH₂—N(CH₃)₂, —NH₂, —NH(CH₃) and —N(CH₃)₂, wherein this group may optionally be substituted by one or more groups selected from among OH, F, Cl, —CF₃, CHF₂, CH₂F, oxo, methyl and phenyl or wherein
R³ denotes a group selected from among a het and hetaryl, which may optionally be substituted by one or more groups selected from among F, Cl, Br, CF₃, CHF₂, CH₂F, CN, OH, oxo, methyl, ethyl, propyl, isopropyl, butyl, isobutyl, cyclopropyl, —O-methyl, —O-ethyl, —O-propyl, —O-isopropyl, —COO-methyl, —COO-ethyl, —COO-propyl, —COO-isopropyl, SO—(CH₃), SO—(CH₂—CH₃), SO₂—(CH₃), SO₂—(CH₂—CH₃), phenyl, CH₂—NH₂, CH₂—NH(CH₃), CH₂—N(CH₃)₂, —NH₂, —NH(CH₃), —N(CH₃)₂, het and hetaryl, which in turn may optionally be substituted by one or more groups selected from among OH, F, Cl, CF₃, CHF₂, CH₂F, methyl, ethyl, propyl, isopropyl, phenyl, —COO-methyl, —COO-ethyl and O-methyl, O-ethyl, or wherein
R³ denotes —O—R^{3.1}, wherein R^{3.1} is a group selected from among —C₁₋₃-alkyl, -phenyl, —C₁₋₃-alkylene-phenyl, hetaryl and het, which is optionally substituted in the ortho, para or meta position by one, two or three groups selected independently of one another from among fluorine, chlorine, bromine, hydroxy, CN, methyl, ethyl, propyl, isopropyl, butyl, isobutyl, CF₃, CHF₂, CH₂F, CO-(methyl), CO-(ethyl), —CO-(propyl), CO-(isopropyl), —CO—(CF₃), —CO—NH-(methylene)-hetaryl, —CO—NH-(ethylene)-hetaryl, —CO—N(CH₃)-(methylene)-hetaryl, —CO—N(CH₃)-(ethylene)-hetaryl, —CO—N(CH₃)-(propylene)-hetaryl, —CO—N(CH₃)-(isopropylene)-hetaryl, —CO—N(CH₃)-het, —CO—N(cyclopropyl)-het, —CO—N(C₅₋₇-cycloalkyl)-het, -methylene-O-methyl, -ethylene-O-methyl, -propylene-O-methyl, -methylene-O-ethyl, -ethylene-O-ethyl, -propylene-O-ethyl, -methylene-NH₂, -methylene-NHCH₃, -methylene-N(CH₃)₂, -ethylene-NH₂, -ethylene-NHCH₃, -ethylene-N(CH₃)₂, NH₂, N(CH₃)₂, NHCH₃, —O-methyl, O-ethyl, O-propyl, O-isopropyl, O-butyl, O-isobutyl, —SO—CH₃, SO-ethyl, —SO-propyl, —SO-isopropyl, SO₂-methyl, —SO₂-ethyl, SO₂-propyl, SO₂-isopropyl, COOH, COO-(methyl), COO-(ethyl), COO-(propyl), COO-(isopropyl), —O-methylene-N(methyl)₂, —O-ethylene-N(methyl)₂, —O-methylene-N(ethyl)₂, —O-ethylene-N(ethyl)₂, CO—NH₂, CO—NH(CH₃), CO—N(CH₃)₂, —NH—CO-methyl, —NCH₃—CO-methyl, —NH—CO-ethyl, NCH₃—CO-ethyl, phenyl, phenyl-methylene, phenyl-ethylene, het-methylene, het-ethylene, —CO-het, het, —CO—C₅₋₇-cycloalkyl, —CO-cyclopropyl, —CO—N(CH₃)—C₅₋₇-cycloalkyl, CO—N(CH₃)-cyclopropyl, C₅₋₇-cycloalkyl, cyclopropyl, C₅₋₇-cycloalkyl-methylene, C₅₋₇-cycloalkyl-ethylene, cyclopropyl-methylene, cyclopropyl-ethylene, hetaryl-methylene, hetaryl-ethylene and hetaryl, which in turn may optionally be substituted by 1, 2, 3 or 4 groups selected independently of one another from among F, Cl, Br, methyl, O-methyl, ethyl, O-ethyl, OH, oxo and CF₃, and wherein
R⁴ denotes H, CN, OH, CF₃, CHF₂, CH₂F, F, methyl, ethyl, O-methyl, O-ethyl, -methylene-OH, -ethylene-OH, -propylene-OH, isopropylene-OH, —COO(methyl), —COO(ethyl), —COO(propyl), —COO(isopropyl), —CO-het, -(methylene)-NH—SO₂-(methyl), -(methylene)-NH—SO₂-(ethyl), -(ethylene)-NH—SO₂-(methyl), -(ethylene)-NH—SO₂-(ethyl), -(methylene)-N(CH₃)—SO₂-(methyl), -(methylene)-N(CH₃)—SO₂-(ethyl), -(ethylene)-N(CH₃)—SO₂-(methyl), -(ethylene)-N(CH₃)—SO₂-(ethyl), -(methylene)-O-(methylene)-phenyl, -(methylene)-O-(ethylene)-phenyl, -(ethylene)-O-(methylene)-phenyl, -(ethylene)-O-(ethylene)-phenyl, -methylene-O-methyl, -methylene-O-ethyl, -ethylene-O-methyl, -ethylene-O-ethyl, -(methylene)-N(CH₃)—CO-(methyl), -(methylene)-N(CH₃)—CO-(ethyl), -(ethylene)-N(CH₃)—CO-(methyl), -(ethylene)-N(CH₃)—CO-(ethyl), —NH—CO-(methylene)-O-(methyl), —NH—CO-(methylene)-O-(ethyl), —NH—CO-(ethylene)-O-(methyl), —NH—CO-(ethylene)-O-(ethyl), -methylene-NH—CO-(methyl), -methylene-NH—CO-(ethyl), -ethylene-NH—CO-(methyl), -ethylene-NH—CO-(ethyl), -methylene-NH—CO-(methylene)-N(methyl)₂, -methylene-NH—CO-(ethylene)-N(methyl)₂, -ethylene-NH—CO-(methylene)-N(methyl)₂, -ethylene-NH—CO-(ethylene)-N(methyl)₂, -methylene-NH—CO-(methylene)-O-(methyl), -methylene-NH—CO-(ethylene)-O-(methyl), -ethylene-NH—CO-(methylene)-O-(methyl), -methylene-NH—CO-(methylene)-O-(ethyl), -methylene-NH—CO-(ethylene)-O-(ethyl), -ethylene-NH—CO-(methylene)-O-(ethyl), -(methylene)-N(CH₃)—CO-(methylene)-O-(methyl), -(methylene)-N(CH₃)—CO-(ethylene)-O-(methyl), -(ethylene)-N(CH₃)—CO-(methylene)-O-(methyl), -(methylene)-N(CH₃)—CO-(methylene)-O-(ethyl), -(methylene)-N(CH₃)—CO-(ethylene)-O-(ethyl), -(ethylene)-N(CH₃)—CO-(methylene)-O-(ethyl), —O-(methylene)-phenyl, —O-(ethylene)-phenyl, —CO-phenyl, wherein the phenyl in the above groups may optionally be substituted by one or more other groups selected from among F, Cl, Br, methyl, ethyl, propyl, —O-methyl, —O-ethyl, —O-propyl, —OH and CF₃ or wherein
R³ and R⁴ together form a mono- or bicyclic, unsaturated, saturated or partly saturated heterocyclic group which contains 1, 2 or 3 heteroatoms selected from among N, O and S and which may optionally be substituted by one or more groups selected from among F, Cl, Br, OH, oxo, CF₃, CHF₂, CH₂F, CN, methyl, ethyl, propyl, isopropyl, cyclopropyl, COO-methyl, —COO-ethyl, O-methyl, O-ethyl, SO₂—(CH₃), SO₂—(CH₂CH₃), SO—(CH₃), SO—(CH₂CH₃), CH₂—NH₂, CH₂—NH(CH₃), CH₂—N(CH₃)₂, —NH₂, —NH(CH₃), —N(CH₃)₂, phenyl, C₅₋₇-cycloalkyl, het and hetaryl,
   as well as pharmacologically acceptable salts, diastereomers, enantiomers, racemates, hydrates or solvates thereof.

Preferably the invention relates to the use of one of the above compounds of formula I for the manufacture of a medicament for the treatment of diabetes mellitus or for the treatment of a microvascular or macrovascular complication of diabetes mellitus, wherein

R² is a group according to formula 3

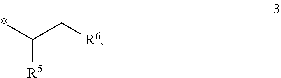

wherein R⁶ is OH or NH₂ and
wherein R⁵ is methyl, ethyl, propyl, isopropyl,
as well as pharmacologically acceptable salts, diastereomers, enantiomers, racemates, hydrates or solvates thereof.

Preferably, the invention refers to the use of one of the above compounds of formula I for the manufacture of a medicament for the treatment of diabetes mellitus or for the treatment of a microvascular or macrovascular complication of diabetes mellitus, wherein
R² is a cyclopropyl or cyclobutyl which may optionally be substituted by another group selected from among OH, —CH₂—OH, —NH₂, CH₂—NH₂, —NH(CH₃), —N(CH₃)₂, methyl, ethyl, propyl, isopropyl, —NH—CO-(tert-butyl), —NH—CO—O-(tert-butyl), —N(CH₃)—CO-(tert-butyl), —N(CH₃)—CO—O-(tert-butyl), —CF₃, —CHF₂, CH₂F, F, Cl and Br
   as well as pharmacologically acceptable salts, diastereomers, enantiomers, racemates, hydrates or solvates thereof.

Preferably, the invention relates to the use of one of the above compounds of formula I for the manufacture of a medicament for the treatment of diabetes mellitus or for the treatment of a microvascular or macrovascular complication of diabetes mellitus, wherein
R² is a phenyl which may optionally be substituted in one or both meta positions by one or more groups selected from among methyl, ethyl, propyl, isopropyl, cyclopropyl, F, Cl, Br, OH, OR^{2.1}, COOR^{2.1}, CF₃, CHF₂, CH₂F, NH₂, NH(CH₃) and N(CH₃)₂, wherein R^{2.1} may be H, methyl or ethyl,
   as well as pharmacologically acceptable salts, diastereomers, enantiomers, racemates, hydrates or solvates thereof.

Preferably, the invention refers to the use of one of the above compounds of formula I for the manufacture of a medicament for the treatment of diabetes mellitus or for the treatment of a microvascular or macrovascular complication of diabetes mellitus, wherein
R² denotes a group selected from among piperidine or tetrahydropyran which may optionally be substituted by one or more groups selected from among F, Cl, Br, OH, CF₃, CHF₂, CH₂F, NH₂, NHCH₃, N(CH₃)₂, oxo, methyl and methoxy,
   as well as pharmacologically acceptable salts, diastereomers, enantiomers, racemates, hydrates or solvates thereof.

In a particularly preferred embodiment the invention relates to the use of one of the above compounds of formula I for the manufacture of a medicament for the treatment of diabetes mellitus or for the treatment of a microvascular or macrovascular complication of diabetes mellitus, wherein the compound of formula I is selected from the group consisting of
1.1 (R)-2-{2-[4-(4-chlorophenyl)-piperidin-1-yl]-5-oxo-6,7-dihydro-5H-5λ⁴-thieno[3,2-d]pyrimidin-4-ylamino}-3-methylbutan-1-ol
1.2 (1-{2-[4-(4-chlorophenyl)-piperidin-1-yl]-5-oxo-6,7-dihydro-5H-5λ⁴-thieno[3,2-d]pyrimidin-4-ylamino}-cyclopropyl)-methanol
1.3 (R)-2-{2-[4-(4-chlorophenyl)-piperidin-1-yl]-5-oxo-6,7-dihydro-5H-5λ⁴-thieno[3,2-d]pyrimidin-4-ylamino}-pentan-1-ol
1.4 (R)-1-{2-[4-(4-chlorophenyl)-piperidin-1-yl]-5-oxo-6,7-dihydro-5H-5λ⁴-thieno[3,2-d]pyrimidin-4-ylamino}-1-(4-fluorophenyl)-2-methylpropan-2-ol
1.5 (S)-5-{2-[4-(4-chlorophenyl)-piperidin-1-yl]-5-oxo-6,7-dihydro-5H-5λ⁴-thieno[3,2-d]pyrimidin-4-ylamino}-1-methylpiperidin-2-one
1.6 {2-[4-(4-chlorophenyl)-piperidin-1-yl]-5-oxo-6,7-dihydro-5H-5λ⁴-thieno[3,2-d]pyrimidin-4-yl}-(tetrahydropyran-4-yl)-amine
1.7 1-(4-(1-hydroxymethylcyclopropylamino)-5-oxo-6,7-dihydro-5H-5λ⁴-thieno[3,2-d]pyrimidin-2-yl)-3′-methyl-1′H-spiro[piperidin-4,4′-quinazolin]-2′(3′H)-one
1.8 {1-[2-(4-benzo[d]isoxazol-3-yl-piperidin-1-yl)-5-oxo-6,7-dihydro-5H-5λ⁴-thieno[3,2-d]pyrimidin-4-ylamino]-cyclopropyl}-methanol
1.9 (1-{2-[4-(2-ethyl-5-fluoro-1H-indol-3-yl)-piperidin-1-yl]-5-oxo-6,7-dihydro-5H-5λ⁴-thieno[3,2-d]pyrimidin-4-ylamino}-cyclopropyl)-methanol
1.10 1-[4-((S)-1-methyl-6-oxopiperidin-3-ylamino)-5-oxo-6,7-dihydro-5H-5λ⁴-thieno[3,2-d]pyrimidin-2-yl]-4-phenylpiperidin-4-carbonitrile
1.11 3′-methyl-1-(4-(tetrahydro-2H-pyran-4-ylamino)-5-oxo-6,7-dihydro-5H-5λ⁴-thieno[3,2-d]pyrimidin-2-yl)-1′H-spiro[piperidin-4,4′-quinazolin]-2′(3′H)-one
1.12 (3-fluorophenyl)-[5-oxo-2-(3,4,5,6-tetrahydro-2H-[4,4′]bipyridinyl-1-yl)-6,7-dihydro-5H-5λ⁴-thieno[3,2-d]pyrimidin-4-yl]amine
1.13 {2-[4-(2-ethyl-5-fluoro-1H-indol-3-yl)-piperidin-1-yl]-5-oxo-6,7-dihydro-5H-5λ⁴-thieno[3,2-d]pyrimidin-4-yl}-(3-fluorophenyl)-amine
1.14 (1-{2-[4-(2,4-difluorophenyl)-piperidin-1-yl]-5-oxo-6,7-dihydro-5H-5λ⁴-thieno[3,2-d]pyrimidin-4-ylamino}-cyclopropyl)-methanol
1.15 {2-[4-(2,4-difluorophenyl)-piperidin-1-yl]-5-oxo-6,7-dihydro-5H-5λ⁴-thieno[3,2-d]pyrimidin-4-yl}-(tetrahydropyran-4-yl)-amine
1.16 (S)-5-[2-(4-benzoxazol-2-yl-piperidin-1-yl)-5-oxo-6,7-dihydro-5H-5λ⁴-thieno[3,2-d]pyrimidin-4-ylamino]-1-methylpiperidin-2-one
1.17 (1-{2-[4-(6-fluorobenzo[d]isoxazol-3-yl)-piperidin-1-yl]-5-oxo-6,7-dihydro-5H-5λ⁴-thieno[3,2-d]pyrimidin-4-ylamino}-cyclopropyl)-methanol
1.18 (1-{2-[4-(5-fluorobenzo[d]isoxazol-3-yl)-piperidin-1-yl]-5-oxo-6,7-dihydro-5H-5λ⁴-thieno[3,2-d]pyrimidin-4-ylamino}-cyclopropyl)-methanol
1.19 {2-[4-(5-furan-2-yl-2H-pyrazol-3-yl)-piperidin-1-yl]-5-oxo-6,7-dihydro-5H-5λ⁴-thieno[3,2-d]pyrimidin-4-yl}-(tetrahydropyran-4-yl)-amine
1.20 (3-fluorophenyl)-{5-oxo-2-[4-(3-pyridin-4-yl-[1,2,4]oxadiazol-5-yl)-piperidin-1-yl]-6,7-dihydro-5H-5λ⁴-thieno[3,2-d]pyrimidin-4-yl}-amine
1.21 (R)-3-methyl-2-{5-oxo-2-[4-(3-pyridin-4-yl-[1,2,4]oxadiazol-5-yl)-piperidin-1-yl]-6,7-dihydro-5H-5λ⁴-thieno[3,2-d]pyrimidin-4-ylamino}-butan-1-ol
1.22 (S)-5-{2-[4-(4-fluorophenoxy)-piperidin-1-yl]-5-oxo-6,7-dihydro-5H-5λ4-thieno[3,2-d]pyrimidin-4-ylamino}-1-methylpiperidin-2-one
1.23 (2-{4-[4-(4,5-dihydrooxazol-2-yl)-phenoxy]-piperidin-1-yl}-5-oxo-6,7-dihydro-5H-5λ⁴-thieno[3,2-d]pyrimidin-4-yl)-(tetrahydropyran-4-yl)-amine
1.24 4-{1-[5-oxo-4-(tetrahydropyran-4-ylamino)-6,7-dihydro-5H-5λ⁴-thieno[3,2-d]pyrimidin-2-yl]-piperidin-4-yloxy}-benzoic acid
1.25 2-(1-{2-[4-(4-chlorophenyl)-piperidin-1-yl]-5-oxo-6,7-dihydro-5H-5λ⁴-thieno[3,2-d]pyrimidin-4-ylamino}-cyclopropyl)-propan-2-ol
1.26 {2-[4-(5-tert-butyl-1-methyl-1H-indol-3-yl)-piperidin-1-yl]-5-oxo-6,7-dihydro-5H-5λ⁴-thieno[3,2-d]pyrimidin-4-yl}-(tetrahydropyran-4-yl)-amine
1.27 2-[4-(5-furan-2-yl-1-methyl-1H-pyrazol-3-yl)-piperidin-1-yl]-5-oxo-6,7-dihydro-5H-5λ⁴-thieno[3,2-d]pyrimidin-4-yl}-(tetrahydropyran-4-yl)-amine
1.28 (S)-5-(2-{4-[4-(4,5-dihydrooxazol-2-yl)-phenoxy]-piperidin-1-yl}-5-oxo-6,7-dihydro-5H-5λ⁴-thieno[3,2-d]pyrimidin-4-ylamino)-1-methylpiperidin-2-one
1.29 {2-[4-(5-furan-2-yl-2-methyl-2H-pyrazol-3-yl)-piperidin-1-yl]-5-oxo-6,7-dihydro-5H-5λ⁴-thieno[3,2-d]pyrimidin-4-yl}-(tetrahydropyran-4-yl)-amine
1.30 {2-[4-(1-methyl-1H-imidazo[4,5-c]pyridin-2-yl)-piperidin-1-yl]-5-oxo-6,7-dihydro-5H-5λ⁴-thieno[3,2-d]pyrimidin-4-yl}-(tetrahydropyran-4-yl)-amine
1.31 2-methoxy-N-{1-[5-oxo-4-(tetrahydropyran-4-ylamino)-6,7-dihydro-5H-5λ⁴-thieno[3,2-d]pyrimidin-2-yl]-4-phenylpiperidin-4-ylmethyl}-acetamide
1.32 N-cyclopropyl-N-methyl-4-{1-[5-oxo-4-(tetrahydropyran-4-ylamino)-6,7-dihydro-5H-5λ⁴-thieno[3,2-d]pyrimidin-2-yl]-piperidin-4-yl}-benzamide
1.33 N-cyclopropyl-N-methyl-4-{1-[5-oxo-4-(tetrahydropyran-4-ylamino)-6,7-dihydro-5H-5λ⁴-thieno[3,2-d]pyrimidin-2-yl]-piperidin-4-yloxy}-benzamide
1.34 {5-oxo-2-[4-(pyridin-4-yloxy)-piperidin-1-yl]-6,7-dihydro-5H-5λ⁴-thieno[3,2-d]pyrimidin-4-yl}-(tetrahydropyran-4-yl)-amine
1.35 {2-[4-(4-chlorophenoxy)-piperidin-1-yl]-5-oxo-6,7-dihydro-5H-5λ⁴-thieno[3,2-d]pyrimidin-4-yl}-(tetrahydropyran-4-yl)-amine
1.36 (S)-1-methyl-5-{2-[4-(5-methyl-4-phenyloxazol-2-yl)-piperidin-1-yl]-5-oxo-6,7-dihydro-5H-5λ⁴-thieno[3,2-d]pyrimidin-4-ylamino}-piperidin-2-one
1.37 (1-{2-[4-(5-methyl-4-phenyloxazol-2-yl)-piperidin-1-yl]-5-oxo-6,7-di hydro-5H-5λ⁴-thieno[3,2-d]pyrimidin-4-ylamino}-cyclopropyl)-methanol
1.38 (S)-5-{2-[4-(4,5-diphenyloxazol-2-yl)-piperidin-1-yl]-5-oxo-6,7-dihydro-5H-5λ⁴-thieno[3,2-d]pyrimidin-4-ylamino}-1-methylpiperidin-2-one
1.39 {4-(4-chlorophenyl)-1-[5-oxo-4-(tetrahydropyran-4-ylamino)-6,7-dihydro-5H-5λ⁴-thieno[3,2-d]pyrimidin-2-yl]-piperidin-4-yl}-methanol
1.40 [1-(2-{4-[5-(4-chlorophenyl)-4-methyloxazol-2-yl]-piperidin-1-yl}-5-oxo-6,7-dihydro-5H-5λ⁴-thieno[3,2-d]pyrimidin-4-ylamino)-cyclopropyl]-methanol
1.41 4-(4-chlorophenyl)-1-[5-oxo-4-(tetrahydropyran-4-ylamino)-6,7-dihydro-5H-5λ⁴-thieno[3,2-d]pyrimidin-2-yl]-piperidin-4-ol
1.42 {2-[4-(4-chlorophenyl)-4-methoxypiperidin-1-yl]-5-oxo-6,7-dihydro-5H-5λ⁴-thieno[3,2-d]pyrimidin-4-yl}-(tetrahydropyran-4-yl)-amine
1.43 4-{1-[4-(1-hydroxymethylcyclopropylamino)-5-oxo-6,7-dihydro-5H-5λ⁴-thieno[3,2-d]pyrimidin-2-yl]-piperidin-4-yloxy}-benzonitrile
1.44 5-oxo-2-[4-(4,5,6,7-tetrahydrobenzoxazol-2-yl)-piperidin-1-yl]-6,7-dihydro-5H-5λ⁴-thieno[3,2-d]pyrimidin-4-yl}-(tetrahydropyran-4-yl)-amine
1.45 (S)-5-{2-[4-(4-chlorophenyl)-piperidin-1-yl]-5,5-dioxo-6,7-dihydro-5H-5λ⁶-thieno[3,2-d]pyrimidin-4-ylamino}-1-methylpiperidin-2-one
1.46 (1-{2-[4-(5-Chloro-pyrimidin-2-yl)-piperidin-1-yl]-5-oxo-6,7-dihydro-5H-5λ⁴-thieno[3,2-d]pyrimidin-4-yl-amino}-cyclobutyl)-methanol and
1.47 (1-{2-[4-(4-Chloro-phenyl)-piperidin-1-yl]-5-oxo-6,7-dihydro-5H-5λ⁴-thieno[3.2-d]pyrimidin-4-yl-amino}-cyclobutyl)-methanol.

In a further particularly preferred embodiment the invention relates to the use of one of the above-mentioned compounds of formula I for the treatment of diabetes mellitus type 1.

In another particularly preferred embodiment the invention relates to the use of one of the above-mentioned compounds of formula I for the treatment of diabetes mellitus type 2.

In further particularly preferred embodiment the invention relates to the use of the above-mentioned compounds of formula I for the treatment of a microvascular complication of diabetes mellitus selected from the group consisting of diabetic retinopathy, diabetic nephropathy, diabetic neuropathy, diabetic foot and diabetic ulcer.

In another particularly preferred embodiment the invention relates to the use of the above-mentioned compounds of formula I for the treatment of a macrovascular complication of diabetes mellitus selected from the group consisting of myocardial infarct, acute coronary syndrome, unstable angina pectoris, stable angina pectoris, stroke, peripheral arterial occlusive disease, cardiomyopathy, heart failure, heart rhythm disorders and vascular restenosis.

### Methods of Synthesis

The compounds 1.1 to 1.45 of general formula (I) may be prepared according to the following syntheses as described in detail in WO 2010/097334 and in WO 2009/050248. However, the compounds 1.46 and 1.47 have been made according to PCT/EP2012066104 and according to the following methods of synthesis:

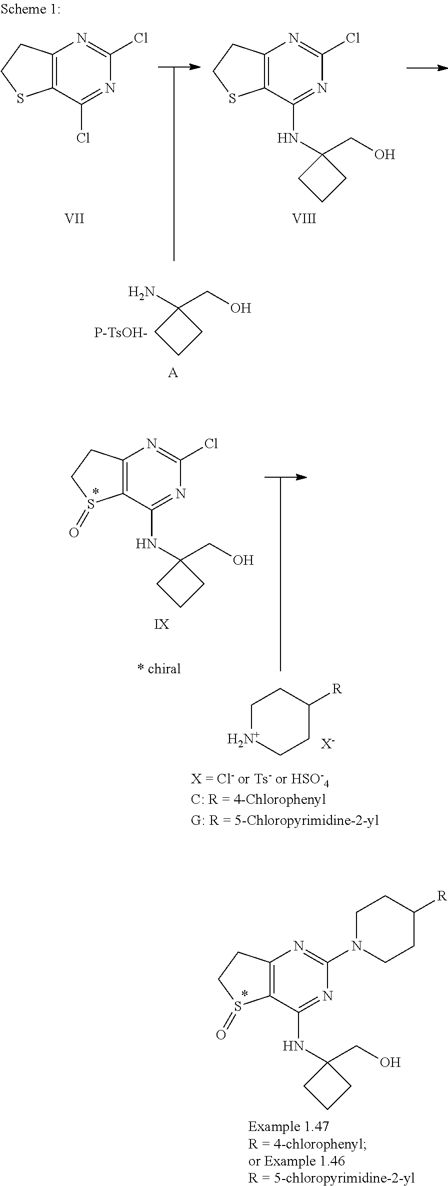

### Generation of Compound VII

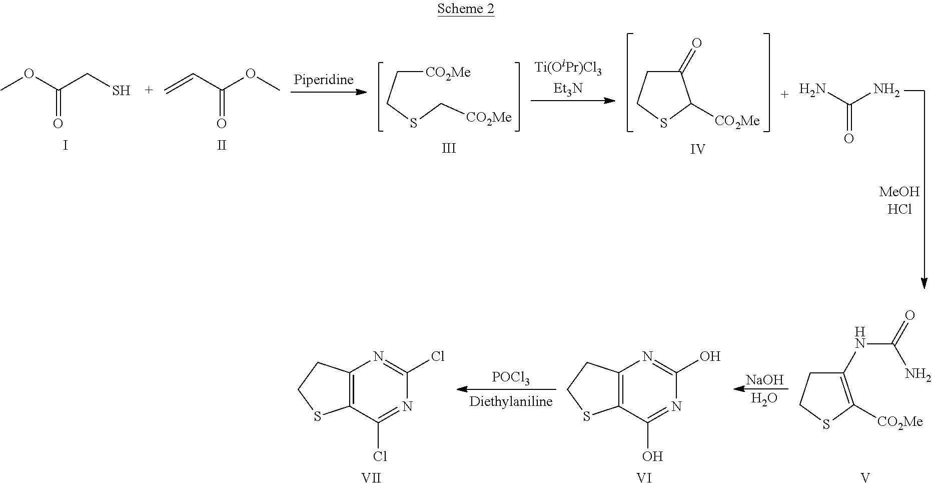

### Synthesis of Dimethyl-3-thiaadipate (Compound III)

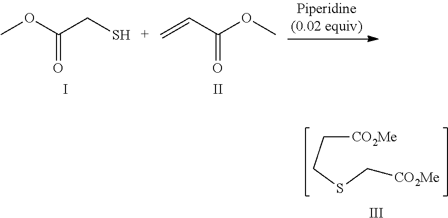

Methyl thioglycolate (292 g, 2.61 mol) and piperidine (4.43 g, 0.052 mol) were charged to an inerted jacketed reactor equipped with an addition funnel, mechanical stirrer, N₂ line and thermocouple thermometer. Methyl acrylate (250 g, 2.87 mol) was then added slowly over a period of 30 min keeping the temperature at approximately 45° C. Upon complete addition, the mixture was stirred at 45° C. for 30 min. Piperidine (17.9 g, 210 mmol) was added and stirring at 45° C. continued for 30 min (in order to scavenge of excess acrylate). Tert-butyl methyl ether (MTBE) (251 ml) was charged, the mixture was cooled to 15° C. and 1 M HCl (251 ml) was added. The mixture was stirred for 5 min and the organic layer was collected and washed with water (251 ml). The mixture was concentrated to a minimum volume by distillation under reduced pressure at 50° C. Dichloromethane (251 ml) was charged and the mixture was again concentrated under reduced pressure by distillation at 40-45° C. Crude product III (480 g) was used in the next step without further purification.

### Synthesis of Methyl-3-oxo-tetrahydrothiophene-2-carboxylate (Compound IV)

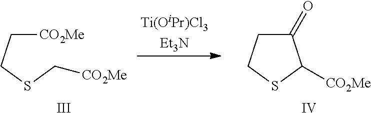

TiCl₄ (1.0 M CH₂Cl₂, 1.16 L; 1.16 mol) was charged to an inerted and dried jacketed reactor equipped with temperature probe, mechanical stirrer and a dropping funnel. The reactor contents were cooled to −10° C. and isopropanol (89.6 ml, 1.16 mol) was charged at or below −10° C. The mixture was stirred at −10° C. for 30 min and dimethyl 3-thiaadipate (200 g, 1.01 mol) was charged slowly over 1 h keeping the internal temperature at or below −10° C. The reaction was stirred for an additional 30 min at −10° C. and Et₃N (489 mL, 3.49 mol) was slowly charged over 1.5 hours keeping the internal temperature at or below −10° C. The mixture was stirred at or below −10° C. for 1.5 hours. 3 N HCl (1.01 L; 3.03 mol) was slowly charged keeping the internal temperature below 10° C. The temperature was increased to 30° C. and the mixture was stirred for 1 hour. The mixture was allowed to settle, the organic layer was collected and the aqueous layer was extracted with dichloromethane twice (1.5 l per extraction). The combined organic portions were washed twice with water (1.5 l per wash) and dried with MgSO₄ (40 g). The resulting solution was concentrated to a minimum volume under reduced pressure at 25-35° C. to afford crude IV (148.6 g). The spectral data of IV is consistent with literature values (Liu, H.-J.; Teng, K. N. Can. J. Chem. 1982, 60, 437).

### Synthesis of 3-Ureido-4,5-dihydro-thiophene-2-carboxylic acid methyl ester (Compound V)

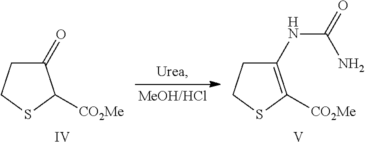

Urea (2.16 kg, 35.9 mol) was charged into a dry, jacketed reactor equipped with a stirrer, N₂ line and thermocouple thermometer. 3-oxo-tetrahydro-thiophene-2-carboxylic acid methyl ester (Compound IV, 3.0 kg) was charged followed by methanol (4.5 l). Conc. HCl (297 ml, 3.59 mol) was charged at 20-25° C. and the mixture stirred at reflux for 4-6 hours. The reaction mixture was cooled to 0° C. and the resulting solid was collected by filtration. The cake was washed with water twice (2 l water per wash) and dried in a vacuum oven at 50° C. to afford 4.17 kg (83% w/w) of compound V (95% yield), ¹H NMR (500 MHz, (CD₃)₂SO) δ 3.10 (dd, 2H, J=8.5, 8.5 Hz), 3.50 (dd, 2H, J=8.5, 8.5 Hz), 3.73 (s, 3H), 6.50-7.20 (bs, 2H), 9.47 (s, 1H); ¹³C NMR (125 MHz, (CD₃)₂SO) δ 28.7, 37.8, 52.4, 100.0, 151.6, 154.7, 165.7; LCMS (EI) for C₇H₁₁N₂O₃S, (M+H)+ calcd. 203.0, measd. 203.0.

### Synthesis of 6,7-Dihydro-thieno[3,2-d]pyrimidine-2,4-diol (Compound VI)

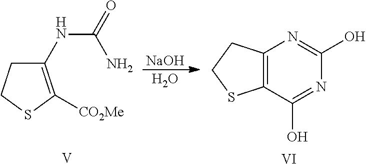

Compound V (2.0 kg, 9.47 mol) was added to a solution of water (6.0 l) and NaOH (379 g, 9.47 mol) at normal room temperature. The above mixture was stirred at 85° C. for 3 hours. After cooling to 0° C., conc. HCl (861 ml, 10.4 mol) was added slowly until the pH of the solution was 0-1. The mixture was cooled to 0° C., stirred for 5-10 min and the resulting solid was collected by filtration. The cake was washed thoroughly with water twice (1 l per rinse), air-dried for 2-3 hours (suction) and then dried further in a vacuum oven at 50° C. for 12-16 hours to afford 1.67 kg of compound VI. ¹H NMR (500 MHz, (CD₃)₂SO) δ 3.11 (dd, 2H, J=8.5, 8.5 Hz), 3.31 (dd, 2H, J=8.5, 8.5 Hz), 11.14 (s, 1H), 11.38 (s, 1H); ¹³C NMR (125 MHz, (CD₃)₂SO) δ 29.3, 35.4, 108.5, 150.5, 152.4, 160.4; LCMS (EI) for C₆H₇N₂O₂S, (M+H)⁺calcd. 171.0, measd. 171.0.

### Synthesis of 2.4-dichloro-thieno[3,2-d]pyrimidine (Compound VII)

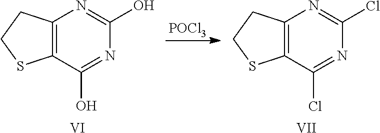

800 g of solid Compound VI (4.66 mol) was charged into to an inert and dry jacketed reactor (reactor 1) equipped with a temperature probe, mechanical stirrer and a dropping funnel. 1.5 liter (9.31 mol) diethylaniline was charged over 30 min to 1 h keeping the temperature at or below 25° C. The internal temperature was brought up to 105-110° C. and 0.68 equiv. (868 ml, 34% of the total) of phosphorus oxychloride was added into the reactor (reactor 1) over 5-10 min. When the inside temperature began to decrease, the internal temperature was maintained at 110° C. and addition of the remaining POCl₃ (1.32 equiv. or 66% of the total) resumed over a period of 30-40 min. The internal temperature was adjusted to 105-110° C. and the mixture was stirred for 18-24 h or until complete (HPLC analysis). The mixture was cooled to 45° C. and THF (400 mL) was charged at 45° C. The above crude mixture was placed into a secondary dry vessel or reactor (vessel or reactor 2). 4.8 l of water was charged into the reactor 1 and cooled to 5° C. The crude reaction mixture (in reactor or vessel 2) is then slowly charged into reactor 1 containing water keeping the temperature at 5-10° C. The mixture was stirred at 5° C. for 30 min to 1 h and the resulting solid was collected by filtration. The cake was rinsed with water twice (1.6 l per rinse) and the cake was air dried in the funnel for 6-8 h to afford 964 g (92% w/w; 88% yield) of crude Compound VII. Dichloromethane (4.6 L) is charged into a 10 L reactor. Crude Compound VII and activated carbon (46.2 g) were charged into the reactor, the mixture is heated to 40° C. and stirred for 20 min. The resulting solution was collected by filtration through a filter media to remove charcoal. The cake was rinsed with dichloromethane twice (175 ml per rinse). The solution was concentrated under reduced pressure to a minimum stirrable volume and the remaining dichloromethane was chased away by distillation with a minimum amount of petroleum ether. Additional petroleum ether (1.3 l) was charged into the reactor, the mixture was cooled to 10° C. and stirred for 1 hr. The resulting solid was collected by filtration and the cake was rinsed with petroleum ether twice (150 ml per rinse). The cake was air dried in the funnel (suction) until it appeared dry. The resulting solid Compound VII was transferred to a suitable tared container and dried in an oven at 50° C. for 6 hours to get final product: ¹H NMR (400 MHz, DMSO-d6) δ 3.45-3.56 (m, 4H); ¹³C NMR (400 MHz, DMSO-d6) δ 29.3, 36.5, 134.8, 151.0, 154.1, 175.9.

### Synthesis of Example 1.47

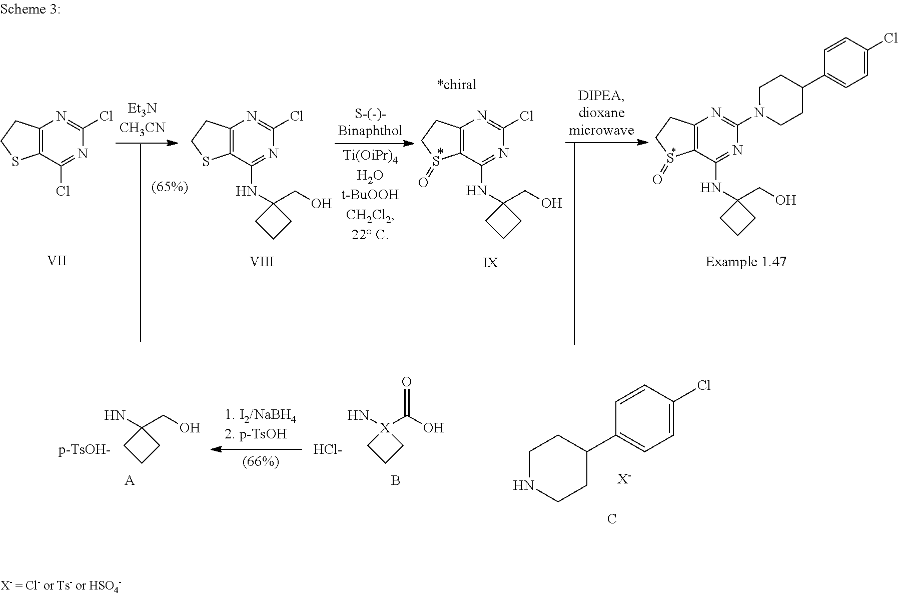

### Synthesis of Compound A

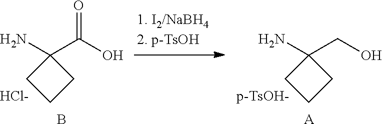

NaBH₄ (28.6 g, 757 mmol, 2.87 eq) and THF (500 ml) were charged to a 2 L reactor under nitrogen and the mixture was cooled to −5° C. A solution of I₂ (63.6 g, 251 mmol, 0.95 eq) in 125 mL THF was prepared and added to the reactor slowly over 45 min maintaining an internal temp of −5 to 5° C. The addition funnel was then rinsed with 42 mL THF. Compound B (50 g, 264 mmol, 1 eq) was then charged at −6° C., then the temperature rose to approx. 5° C. The reaction mixture was then heated to 65° C. for 23 h (Note: Reaction conversion was analyzed by GC/FID by quenching 0.1 mL reaction mixture with MeOH, then derivatizing with 0.5 mL of a 5/2/2 mixture of THF/acetic anhydride/TEA). 83 mL MeOH were then charged to the reaction mixture slowly over 20 min maintaining the temperature between 20-27° C. The reaction mixture was concentrated to a minimum stirrable volume and 500 mL 2-methyltetrahydrofurane (MeTHF) were added. 485 g of 25 wt % aq. NaOH (11.5 eq) were then added, solids were dissolved. The layers were separated and the aqueous phase was extracted twice with 500 ml 2-methyltetrahydrofurane (MeTHF). The organics were then filtered through a pad of CELITE and MgSO₄ and rinsed with 50 mL 2-methyltetrahydrofurane (MeTHF). A solution of p-toluenesulfonic acid monohydrate (51 g, 264 mmol, 1 eq) in MeTHF (100 ml) was prepared and added to the organics (alternatively HCl may be used to obtain the HCl-salt of compound A). A homogeneous light yellow solution resulted. The solution was concentrated to ˜275-300 mL and the water content was checked. Additional MeTHF was added and concentrated to the original volume until the water content was <0.1%. The resulting solid was filtered and rinsed with 50 ml MeTHF, left to dry in the funnel overnight and then dried further in the vacuum oven at 50° C. 61.71 g of compound A were collected: ¹H NMR (DMSO-d6, 400 MHz) δ 1.70-1.92 (m, 2H), 1.94-2.03 (m, 2H), 2.04-2.18 (m, 2H), 2.29 (s, 3H), 3.55 (s, 3H), 5.47 (br s, 1H), 7.13 (d, J=8.0 Hz, 2H), 7.49 (d, J=8.0 Hz, 2H), 7.95 (br s, 3H); ¹³C NMR (DMSO-d6, 100 MHz) δ 13.3, 20.8, 56.4, 63.5, 125.5, 128.1, 137.8, 145.4

### Synthesis of Compound VIII

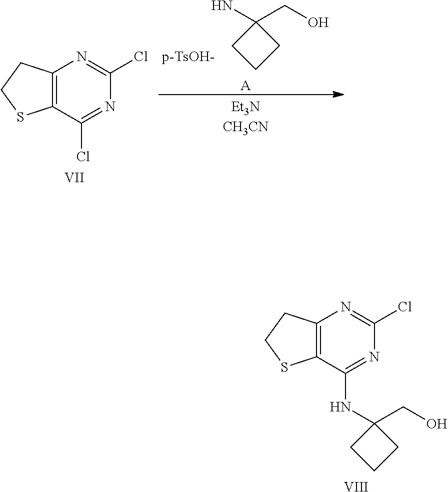

Intermediates VII (180 g, 852 mmol) and A (129 g, 937 mol) were sequentially charged into a multi-neck vessel equipped with a condenser, thermocouple thermometer and nitrogen line. Acetonitrile (900 ml) and triethylamine (594 ml, 4.26 mol) were then added at 22° C. and the mixture was stirred at 75-77° C. for 12 h. Water (1.2 l) was charged slowly over 20 min, the mixture was seeded with Compound VIII crystals (0.3 g) at 40° C. and then cooled to 25° C. over 2 h. The mixture was stirred for an additional 12 h at normal room temperature and the resulting solid was collected by filtration. The filter cake was rinsed with 2:1 mixture of water/MeCN (400 mL) followed by water (200 ml). The resulting solid was dried under vacuum at 50° C. for 12 h to afford 132 g (57% yield) of compound VIII: ¹H NMR (400 MHz, CDCl₃) δ 1.85-2.05 (m, 2H), 2.10-2.21 (m, 2H), 2.32-2.41 (m, 2H), 3.27 (dd, J=8.0, 8.4 Hz, 2H), 3.43 (dd, J=8.0, 8.4 Hz, 2H), 3.91 (s, 2H), 4.67 (s, 1H); ¹³C NMR (CDCl₃, 100 MHz) δ 14.8, 30.7, 31.2, 36.7, 59.7, 67.6, 114.7, 156.1, 156.2, 168.0.

### Synthesis of Compound IX

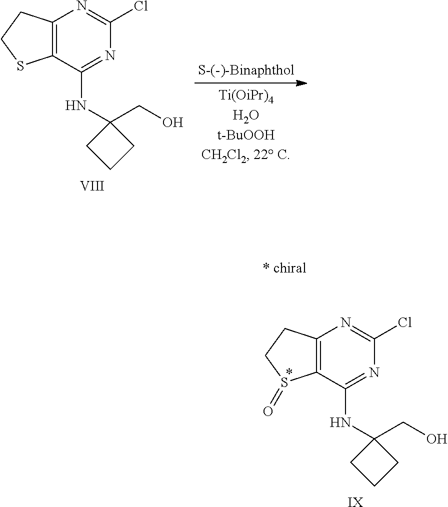

Compound VIII (122 g, 429 mmol), S-(−)-1,1′-Bi-2-naphthol (S-(−)-BINOL) (12.4 g, 42.9 mmol), dichloromethane (608 mL), Ti(OiPr)₄ (6.54 mL, 21.4 mmol), and water (7.72 ml, 429 mmol) were charged to a 2 l multi-neck flask at 20° C. under nitrogen and stirred for 1 h. tert-Butyl hydroperoxide (70% in water, 62.3 ml, 472 mmol) was added at once at 21° C.; the mixture became completely homogeneous and the temperature rose to approx. 40° C. The mixture was allowed to reach normal room temperature, was stirred for 1.5 h and filtered. The cake was twice rinsed with isopropyl acetate (243 ml per rinse) and the cake was air-dried in the filter for >6 h to afford 114.4 g of compound IX. ¹H NMR (400 MHz, DMSO-d6) δ 1.70-1.85 (m, 2H), 2.14-2.34 (m, 4H), 2.98-3.08 (m, 1H), 3.09-3.19 (m, 1H), 3.30-3.40 (obscured m, 1H), 3.50-3.62 (m, 1H), 3.65-3.77 (m, 2H), 4.91 (t, J=6 Hz, 1H), 8.63 (s, 1H); ¹³C NMR (100 MHz, DMSO-d6) δ 14.5, 29.6, 29.8, 32.6, 48.6, 59.2, 62.8, 119.0, 157.8, 161.4, 175.3.

The other enantiomer of compound IX may be produced when S-(−)-1,1′-Bi-2-naphthol is replaced by R-(+)-1,1′-Bi-2-naphthol. A racemate of compound IX may be produced methods known by those skilled in the art that exclude chiral agents and conditions. An example for such a procedure to produce racemic sulfoxides is given in WO 06/111549.

### Synthesis of Example 1.47

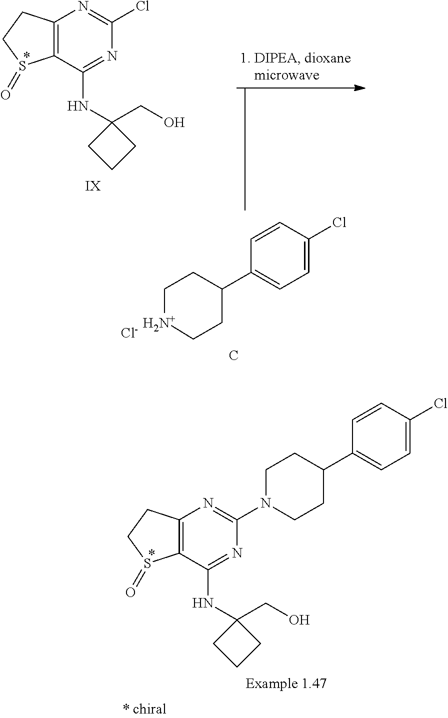

Sulfoxide IX (6.48 g; 22.5 mmol), 4-(4-Chlorophenyl)-piperidine hydrochloride C (5.75 g; 24.8 mmol) (alternatively the p-TsOH-salt or the H₂SO₄-salt of compound C) and N,N-diisopropylethylamine (12.4 ml; 72.1 mmol) were mixed in 47 ml of dioxane. The resulting mixture was charged to three 20 ml vials which were heated to 120° C. for 25 min. in a microwave oven. After cooling to room temperature, the reaction mixtures were poured on ice water. The resulting precipitate was filtered off, taken up in 500 ml ethyl acetate and heated to reflux. After refluxing, the mixture was cooled in an ice bath and the resulting precipitate was filtered off and dried in a dry box at 50° C. at reduced pressure yielding 7.57 g of Example 1.47. ¹H NMR (400 MHz, DMSO-d6) δ 1.43-1.57 (m, 2H), 1.67-1.85 (m, 4H), 2.11-2.21 (m, 2H), 2.26-2.43 (m, 2H), 2.80-3.01 (m, 5H), 3.17-3.47 (m, integration compromised by water peak), 3.67-3.76 (m, 2H), 4.74-4.86 (m, 3H), 7.25-7.36 (m, 5H). ¹³C NMR (100 MHz, DMSO-d6) δ 14.3, 29.4, 29.6, 32.3, 32.5, 41.4, 44.2, 48.5, 58.4, 63.6, 109.2, 128.2, 128.6, 130.5, 144.7, 157.6, 161.5, 174.7.

### Generation of Example 1.46

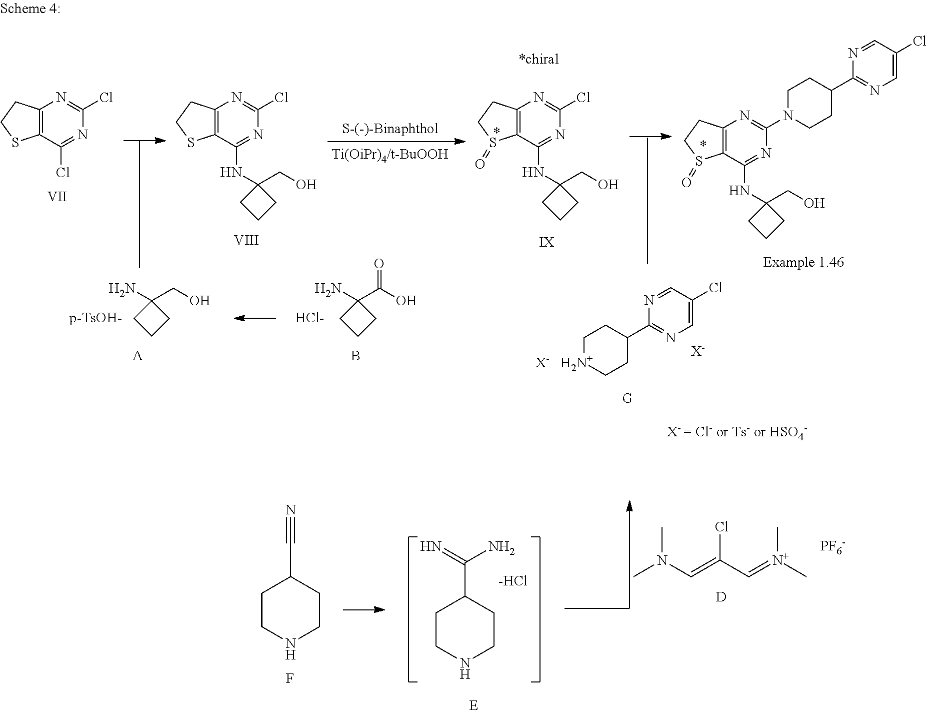

### Generation of Compound G

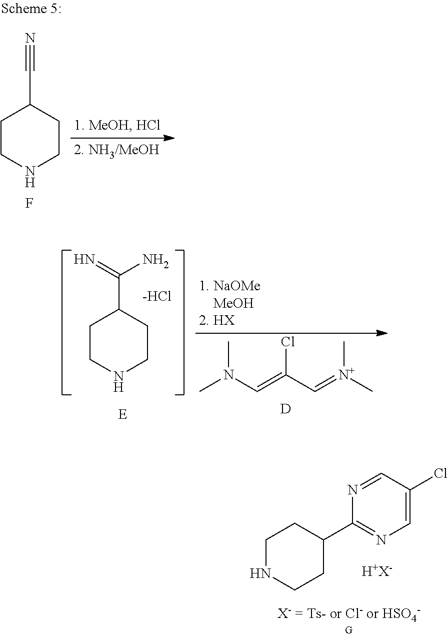

### Synthesis of Compound E

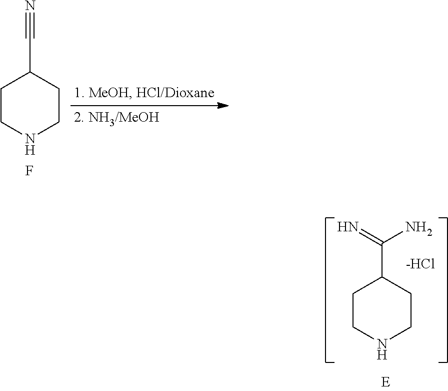

4M HCl in dioxane (225 ml, 3 eq, 900 mmol) was charged to a 500 ml 3-neck jacketed reactor equipped with a mechanical stirrer, temperature probe and argon line. The solution was cooled to 0° C. and 4-cyanopiperidine (33.04 g, 300 mmol) was charged followed by methanol (36.4 ml, 900 mmol, 3 equiv) over ˜30 min while keeping the temperature below 10° C. (temperature rose). The above mixture was stirred for 6-8 h at normal room temperature until complete conversion was observed by ¹H NMR analysis of an aliquot in D₂O (the clear solution turned into a white slurry after 30 min). The mixture was cooled to 5° C. and 25 wt % NaOMe in methanol (129.6 g, 600 mmol, 2 eq) was charged while maintaining the temperature below 15° C. The mixture was then stirred for 1 h. 7.0 N ammonia in methanol (64.2 ml, 1.5 eq, 450 mmol) was charged to the above mixture and stirred for 2 h at normal room temperature. The mixture was concentrated under reduced pressure at 60° C. to a volume of ˜250 ml to afford a solution of crude compound E that was used without isolation: ¹H NMR (400 MHz, D₂O) δ 1.80-1.95 (m, 2H), 2.15 (br d, J=4.4 Hz, 2H), 2.79-2.90 (m, 1H), 3.02 (ddd, J=13.2, 13.2, 3.0 Hz, 2H), 3.48 (m, 2H).

### Synthesis of Compound G

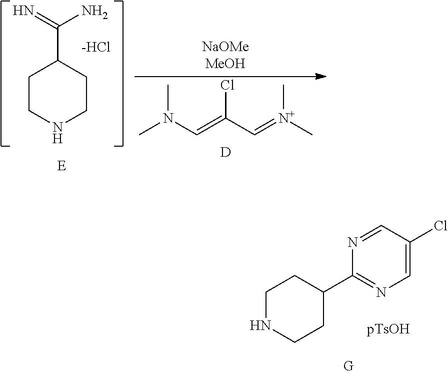

The above solution of intermediate compound E was cooled to ˜20° C. and 25 wt % NaOMe in methanol (162 g, 2.5 eq, 750 mmol) was charged. The mixture was then stirred for 30 min. Compound D (=(Z)—N-(2-chloro-3-(dimethylamino)allylidene)-N-methylmethan-aminium hexafluorophosphate (V)), (82.3 g of 95 wt % purity, 0.85 eq, 255 mmol) was charged to the above mixture in two portions at normal room temperature over ˜30 min and stirred for 3 h at room temperature. The mixture was concentrated under reduced pressure at 60° C. to a volume of ˜200 ml. 2-Methyltetrahydrofuran (400 ml) was charged and the mixture was concentrated further to a volume of ˜150 ml under reduced pressure at 60° C. 2-methyltetrahydrofuran (250 ml) was charged, the mixture was cooled to ˜20° C., water (150 ml) was added and the mixture was stirred for 5 min. The layers were separated and the organic layer was collected. The organic layer was washed with 30% aqueous NaOH (120 ml) and the layers were separated. The organics were concentrated to a minimum stirrable volume (˜150 mL) and n-propanol (350 ml) was charged. A solution of p-toluenesulfonic acid monohydrate in n-propanol (0.85 equiv., 255 mmol, 48.4 g in 100 ml n-propanol) was charged to the above clear solution over 10 min at ˜65° C. The above mixture was concentrated at ˜65° C. under reduced pressure to maintain ˜350 ml and <1.0% water (it is recommended to have a water content below 1.0% to avoid product losses to the mother liquor). The batch was cooled to 20° C. with stirring over 3 h. The solids were filtered, rinsed with the filtrate and then with n-propanol (120 mL) to afford 111 g (68% w/w by assay, 75.48 g) of compound G after vacuum drying at 65° C. in a vacuum oven for 12 h. ¹H NMR (DMSO-d6, 400 MHz) δ 1.83-1.99 (m, 2H), 2.13 (d, J=12 Hz, 2H), 2.97 (s, 3H), 3.0-3.11 (m, 2H), 3.13-3.23 (m, 1H), 3.30-3.42 (m, 2H), 7.14 (d, J=8.0 Hz, 2H), 7.52 (d, J=8.0 Hz, 2H), 8.47 (br, 2H), 8.91 (s, 2H); ¹³C NMR (DMSO-d6, 100 MHz) δ 20.7, 27.0, 40.8, 42.8, 125.5, 128.1, 128.8, 137.9, 145.2, 155.8, 169.0.

### Synthesis of Example 1.46

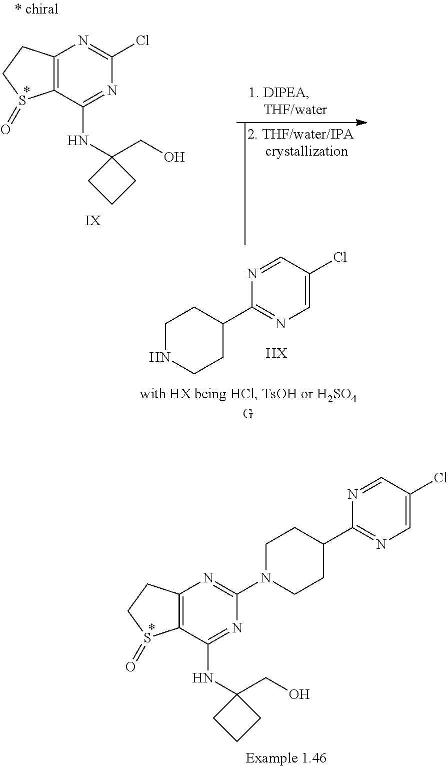

Compound IX (86.5 g, 291 mmol, 1 eq), compound G (160 g, 305 mmol, 1.05 eq), tetrahydrofuran (THF) (484 ml), water (121 ml) and DIPEA (N,N-diisopropylethylamine, 127 ml, 727 mmol, 2.5 eq) were all charged to a 3 l round bottom flask under nitrogen and heated to 65° C. for 3 h. Water (1125 ml, 13 ml/g compound IX) was then charged at the temperature 65° C. and stirred for 2 h while cooling to 20° C. The mixture was filtered and the cake was washed twice with 173 ml acetone. The cake was then left to dry on the funnel overnight to afford 116.7 g of Example 1.46: ¹H NMR (400 MHz, CDCl₃) δ 1.75-1.95 (m, 4H), 2.02-2.11 (m, 2H), 2.12-2.26 (m, 2H), 2.38 (q, J=9.6 Hz, 2H), 2.93-3.12 (m, 4H), 3.12-3.22 (m, 1H), 3.28-3.39 (m, 1H), 3.53-3.65 (m, 1H), 3.80 (d, J=5.6 Hz, 2H), 4.42 (t, J=5.2 Hz, 1H), 4.82 (br d, J=11.2 Hz, 2H), 6.47 (s, 1H), 8.62 (s, 2H); ¹³C NMR (100 MHz, CDCl₃) δ 14.8, 30.0, 30.1, 30.6, 32.7, 44.3, 49.4, 59.1, 68.2, 107.5, 129.1, 155.5, 159.0, 162.3, 170.5, 174.6.

### Biological Experiments

### Determination of the PDE4B IC₅₀-Values (In Vitro)

The IC₅₀-values of the compounds of the invention (Examples 1.1 to 1.47) with respect to their PDE4B-inhibiting ability have been determined with a Scintillation Proximity (SPA) Assay (GE Healthcare, No. TRKQ7090). The Scintillation Proximity (SPA) Assay is based on the detection of the different affinities of the cyclic 3′-5′adenosine monophosphate (cAMP, low affinity) and the linear 5′-adenosine monophosphate (AMP, high affinity) to yttrium-silicate-scintillator beads. The cAMP-specific phosphodiesterase (PDE) PDE4B cleaves the 3′-phosphodiester bond of the tritium-labelled-[³H]cAMP to the [³H]5′-AMP. This [³H]5′-AMP associates with the scintillator beads because of their higher affinity and causes scintillations (light flashes) which can be measured in a Wallac Microbeta Scintillation Counter.

10 μl of a [³H]cAMP-solution (0.05 μCi in H₂O, 10-30 Ci/mmol) are added to 89 μl of a PDE4B-enzyme-solution (active site fragment comprising the amino acids 152-484; 0.15-0.18 ng) in assay buffer (50 mM Tris HCl pH 7.5; 8.3 mM MgCl₂; 1,7 mM ethylene glyclol tetraacetic acid (EGTA); 0.25 mg/ml bovine serum albumin (BSA)) and this mixture is incubated at 30° C. for one hour

a) without the compound to be tested (in the presence of 1 μl dimethylsulfoxide (DMSO), corresponding to 1% DMSO) and
b) in the presence of the compound to be tested in a concentration of 125 μM, 25 μM, 5 μM, 1 μM, 200 nM, 40 nM, 8 nM, 1.6 nM, 0.32 nM, 0.064 nM, 0.0128 nM (dilution series in 5er-steps beginning from 125 μM until 0.0128 nM, in the presence of 1% DMSO).

After this incubation the reaction is stopped by the addition of 50 μl of bead-solution (500 mg beads/35 ml H₂O, 18 mM zinc sulfate). In the following 45 minutes the beads have the opportunity to form a sediment. After that the scintillations are measured in the scintillation counter. If the tested compound is able to inhibit the enzymatic activity of the PDE4B-enzyme, less [³H]AMP depending on the concentration of the tested compound is produced and less scintillations are measurable. These results are expressed as IC₅₀-values. The IC₅₀-value stands for the compound concentration at which the PDE4B enzyme activity is inhibited to a half maximal value. Therefore the lower the IC₅₀-value is the better is the PDE4B inhibition.

TABLE 1
Experimentally determined IC₅₀-values
with respect to PDE4B inhibition for the Example
compounds of the invention 1.1 to 1.47

| | Experimentally |
|---|---|
| | determined IC₅₀-value for |
| Example | PDE4B inhibition [μM] |
| 1.1 | 0.0242 |
| 1.2 | 0.0034 |
| 1.3 | 0.0083 |
| 1.4 | 0.0153 |
| 1.5 | 0.0006 |
| 1.6 | 0.0047 |
| 1.7 | >10 |
| 1.8 | 0.0019 |
| 1.9 | 0.0538 |
| 1.10 | 0.0035 |
| 1.11 | >10 |
| 1.12 | >10 |
| 1.13 | >10 |
| 1.14 | 0.0160 |
| 1.15 | 0.0154 |
| 1.16 | 0.0001 |
| 1.17 | 0.00273 |
| 1.18 | 0.00189 |
| 1.19 | 0.00346 |
| 1.20 | 0.0027 |
| 1.21 | >10 |
| 1.22 | 0.0023 |
| 1.23 | 0.0017 |
| 1.24 | 0.007 |
| 1.25 | 0.0018 |
| 1.26 | 0.0380 |
| 1.27 | 0.0210 |
| 1.28 | 0.00019 |
| 1.29 | 0.0017 |
| 1.30 | 0.0334 |
| 1.31 | 0.0057 |
| 1.32 | 0.0085 |
| 1.33 | 0.0075 |
| 1.34 | 0.0080 |
| 1.35 | 0.0039 |
| 1.36 | 0.0004 |
| 1.37 | 0.0035 |
| 1.38 | 0.0002 |
| 1.39 | 0.0018 |
| 1.40 | 0.0006 |
| 1.41 | 0.0067 |
| 1.42 | 0.0038 |
| 1.43 | 0.0035 |
| 1.44 | 0.0027 |
| 1.45 | 0.0540 |
| 1.46 | 0.0072 |
| 1.47 | 0.0043 |

### Beneficial Effect on Glycemic Control of PDE4-Inhibitors

The following example shows the beneficial effect on glycemic control of two different PDE4 inhibitors according to the present invention. All experimental protocols concerning the use of laboratory animals are reviewed by a federal Ethics Committee and approved by governmental authorities.

An oral glucose tolerance test is performed in overnight fasted male Sprague Dawley (SD) rats (RjHan:SD), with a weight of 320-350 g. A pre-dose blood sample is obtained by tail bleed. Blood glucose is measured with a glucometer, and the animals are randomized for blood glucose (n=6/group). Subsequently, the groups receive a single oral administration of either vehicle alone (0.5% aqueous hydroxyethyl-cellulose) or vehicle containing one of the PDE4 inhibitors. The animals receive an oral glucose load (2 g/kg) 60 min after compound administration. Blood glucose is measured in tail blood 15 min, 30 min, 60 min, 90 min, and 120 min after the glucose challenge. Glucose excursion is quantified by calculating the reactive glucose AUC (area under the curve). The data are presented as mean±SEM (standard error of mean). The two-sided unpaired Student t-test is used for statistical comparison of the control group and the active groups.

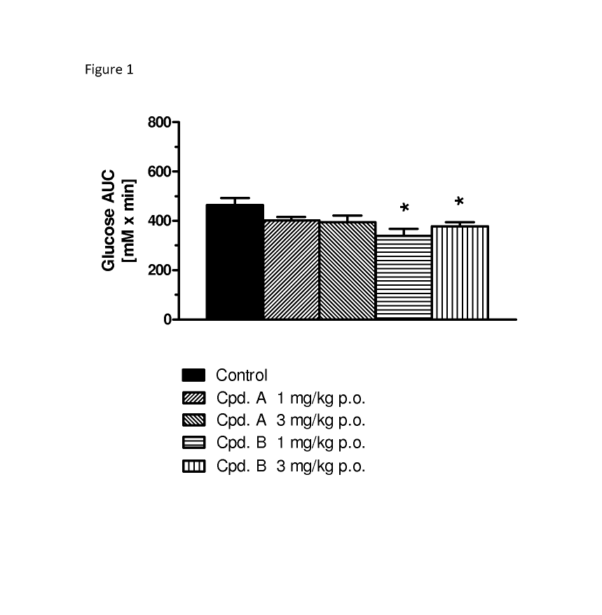

The result is shown in FIG. 1. “Cpd. A” is the PDE4 inhibitor Example 1.35 at a dose of 1 and 3 mg/kg. “Cpd. B” is the PDE4 inhibitor Example 1.46 at a dose of 1 and 3 mg/kg. P values versus control are indicated by symbols above the bars (*, p<0.05). Cpd. A (=Example 1.35) reduces glucose excursion by 13.2 (p=0.09) and 15.0% (p=0.11) with a dose of 1 and 3 mg/kg, respectively. Cpd. B (=Example 1.46) reduces glucose excursion by 26.8% and 18.4% with a dose of 1 and 3 mg/kg, respectively. Cpd. B (=Example 1.46) decreased glucose excursion in the oral glucose tolerance test statistically significant versus control group.

### Combinations

As different metabolic functional disorders often occur simultaneously with diabetes mellitus, it is quite often indicated to combine a number of different active principles with one another. Thus, depending on the functional disorders diagnosed, improved treatment outcomes may be obtained if the compound of formula I is combined with one or more active substances customary for the respective disorders, such as e.g. one or more active substances selected from among the other antidiabetic substances, especially active substances that lower the blood sugar level or the lipid level in the blood, raise the HDL level in the blood, lower blood pressure or are indicated in the treatment of atherosclerosis or obesity.

The PDE4B-inhibitors according to formula I—besides their use in mono-therapy—may also be used in conjunction with other active substances, by means of which improved treatment results can be obtained. Such a combined treatment may be given as a free combination of the substances or in the form of a fixed combination, for example in a tablet or capsule. Pharmaceutical formulations of the combination partner needed for this may either be obtained commercially as pharmaceutical compositions or may be formulated by the skilled man using conventional methods. The active substances which may be obtained commercially as pharmaceutical compositions are described in numerous places in the prior art, for example in the list of drugs that appears annually, the “Rote Liste®” of the federal association of the pharmaceutical industry, or in the annually updated compilation of manufacturers' information on prescription drugs known as the “Physicians' Desk Reference”.

Examples of antidiabetic combination partners are metformin; sulphonylureas such as glibenclamide, tolbutamide, glimepiride, glipizide, gliquidon, glibornuride and gliclazide; nateglinide; repaglinide; mitiglinide; thiazolidinediones such as rosiglitazone and pioglitazone; dipeptidylpeptidase 4 inhibitors (DPP4 inhibitors) such as sitagliptin, saxagliptin, vildagliptin and alogliptin, peroxisome-proliferator-activated receptor gamma modulator (PPAR gamma modulators) such as metaglidases; peroxisome-proliferator-activated receptor gamma agonists (PPAR-gamma agonists) such as e.g. rivoglitazone, mitoglitazone, INT-131 and balaglitazone; peroxisome-proliferator-activated receptor gamma antagonists (PPAR-gamma antagonists); PPAR-gamma/alpha modulators such as tesaglitazar, muraglitazar, aleglitazar, indeglitazar and KRP297; PPAR-gamma/alpha/delta modulators such as e.g. lobeglitazone; AMPK-activators such as AICAR; acetyl-CoA carboxylase (ACC1 and ACC2) inhibitors; diacylglycerol-acetyltransferase (DGAT) inhibitors; pancreatic beta cell G-protein coupled receptor agonists (GCRP agonists) such as GPR119 agonists (SMT3-receptor-agonists); 11 beta-hydroxysteroiddehydrogenase inhibitors (11β-HSD-inhibitors); FGF19 agonists or analogues; alpha-glucosidase blockers such as acarbose, voglibose and miglitol; alpha2-antagonists; insulin and insulin analogues such as human insulin, insulin lispro, insulin glusilin, r-DNA-insulinaspart, NPH insulin, insulin detemir, insulin degludec, insulin tregopil, insulin zinc suspension and insulin glargin; Gastric inhibitory peptide (GIP); amylin and amylin analogues (e.g. pramlintide or davalintide); glucagon-like peptide analogues (GLP-1 and GLP-1 analogues) such as Exendin-4, e.g. exenatide, exenatide LAR, liraglutide, taspoglutide, lixisenatide (AVE-0010), LY-2428757 (a PEGylated version of GLP-1), dulaglutide (LY-2189265), semaglutide or albiglutide; SGLT2-inhibitors such as e.g. dapagliflozin, sergliflozin (KGT-1251), atigliflozin, canagliflozin, ipragliflozin, luseogliflozin or tofogliflozin; inhibitors of protein tyrosine-phosphatase (e.g. trodusquemine); inhibitors of glucose-6-phosphatase; fructose-1,6-bisphosphatase modulators; glycogen phosphorylase modulators; glucagon receptor antagonists; phosphoenolpyruvatecarboxykinase (PEPCK) inhibitors; pyruvate dehydrogenasekinase (PDK) inhibitors; inhibitors of tyrosine-kinases (50 mg to 600 mg) such as PDGF-receptor-kinase (cf. EP-A-564409, WO 98/35958, U.S. Pat. No. 5,093,330, WO 2004/005281, and WO 2006/041976) or of serine/threonine kinases; glucokinase/regulatory protein modulators incl. glucokinase activators; glycogen synthase kinase inhibitors; inhibitors of the SH2-domain-containing inositol 5-s phosphatase type 2 (SHIP2); IKK inhibitors such as high-dose salicylate; JNK1 inhibitors; protein kinase C-theta inhibitors; beta 3 agonists such as ritobegron, YM 178, solabegron, talibegron, N-5984, GRC-1087, rafabegron, FMP825; aldosereductase inhibitors such as AS 3201, zenarestat, fidarestat, epalrestat, ranirestat, NZ-314, CP-744809, and CT-112; SGLT-1 or SGLT-2 inhibitors; KV 1.3 channel inhibitors; GPR40 modulators such as e.g. [(3S)-6-({2′,6′-dimethyl-4′-[3-(methylsulfonyl)propoxy]biphenyl-3-yl}methoxy)-2,3-dihydro-1-benzofuran-3-yl]acetic acid; SCD-1 inhibitors; CCR-2 antagonists; dopamine receptor agonists (bromocriptine mesylate [Cycloset]); 4-(3-(2,6-dimethylbenzyloxy)phenyl)-4-oxobutanoic acid; sirtuin stimulants; and other DPP IV inhibitors.

Examples of combination partners that lower the lipid level in the blood are HMG-CoA-reductase inhibitors such as simvastatin, atorvastatin, lovastatin, fluvastatin, pravastatin, pitavastatin and rosuvastatin; fibrates such as bezafibrate, fenofibrate, clofibrate, gemfibrozil, etofibrate and etofyllinclofibrate; nicotinic acid and the derivatives thereof such as acipimox; PPAR-alpha agonists; PPAR-delta agonists such as e.g. {4-[(R)-2-ethoxy-3-(4-trifluoromethyl-phenoxy)-propylsulfanyl]-2-methyl-phenoxy}-acetic acid; inhibitors of acyl-coenzyme A:cholesterolacyltransferase (ACAT; EC 2.3.1.26) such as avasimibe; cholesterol resorption inhibitors such as ezetimib; substances that bind to bile acid, such as cholestyramine, colestipol and colesevelam; inhibitors of bile acid transport; HDL modulating active substances such as D4F, reverse D4F, LXR modulating active substances and FXR modulating active substances; CETP inhibitors such as torcetrapib, JTT-705 (dalcetrapib) or compound 12 from WO 2007/005572 (anacetrapib); LDL receptor modulators; MTP inhibitors (e.g. lomitapide); PCSK9 inhibitors and ApoB100 antisense RNA.

Examples of combination partners that lower blood pressure are beta-blockers such as atenolol, bisoprolol, celiprolol, metoprolol and carvedilol; diuretics such as hydrochlorothiazide, chlortalidon, xipamide, furosemide, piretanide, torasemide, spironolactone, eplerenone, amiloride and triamterene; calcium channel blockers such as amlodipine, nifedipine, nitrendipine, nisoldipine, nicardipine, felodipine, lacidipine, lercanipidine, manidipine, isradipine, nilvadipine, verapamil, gallopamil and diltiazem; ACE inhibitors such as ramipril, lisinopril, cilazapril, quinapril, captopril, enalapril, benazepril, perindopril, fosinopril and trandolapril; as well as angiotensin II receptor blockers (ARBs) such as telmisartan, candesartan, valsartan, losartan, irbesartan, olmesartan, azilsartan and eprosartan.

Examples of combination partners which increase the HDL level in the blood are Cholesteryl Ester Transfer Protein (CETP) inhibitors; inhibitors of endothelial lipase; regulators of ABC1; LXRalpha antagonists; LXRbeta agonists; PPAR-delta agonists; LXRalpha/beta regulators, and substances that increase the expression and/or plasma concentration of apolipoprotein A-I.

Examples of combination partners for the treatment of obesity are sibutramine; tetrahydrolipstatin (orlistat); alizyme (cetilistat); dexfenfluramine; axokine; cannabinoid receptor 1 antagonists such as the CB1 antagonist rimonobant; MCH-1 receptor antagonists; MC4 receptor agonists; NPY5 as well as NPY2 antagonists (e.g. velneperit); beta3-AR agonists such as SB-418790 and AD-9677; 5HT2c receptor agonists such as APD 356 (lorcaserin); myostatin inhibitors; Acrp30 and adiponectin; steroyl CoA desaturase (SCD1) inhibitors; fatty acid synthase (FAS) inhibitors; CCK receptor agonists; Ghrelin receptor modulators; Pyy 3-36; orexin receptor antagonists; and tesofensine; as well as the dual combinations bupropion/naltrexone, bupropion/zonisamide, topiramate/phentermine and pramlintide/metreleptin.

Examples of combination partners for the treatment of atherosclerosis are phospholipase A2 inhibitors; inhibitors of tyrosine-kinases (50 mg to 600 mg) such as PDGF-receptor-kinase (cf. EP-A-564409, WO 98/35958, U.S. Pat. No. 5,093,330, WO 2004/005281, and WO 2006/041976); oxLDL antibodies and oxLDL vaccines; apoA-1 Milano; ASA; and VCAM-1 inhibitors.

The compounds of formula I may be combined preferably with an active agents selected from the group consisting of metformin, sulphonylureas, nateglinide, repaglinide, thiazolidinediones, dipeptidylpeptidase 4 inhibitors (DPP4-inhibitors), peroxisome proliferator-activated receptor gamma agonists (PPAR-gamma-agonists), alpha-glucosidase inhibitors, insulin, insulin analogues, glucagon-like-peptide 1 (GLP-1) and glucagon-like-peptide 1 analogues (GLP1-analogues).

1. A method of treating diabetes mellitus or a microvascular or macrovascular complication of diabetes mellitus in a patient in need thereof, the method comprising administering to the patient a compound of formula 1 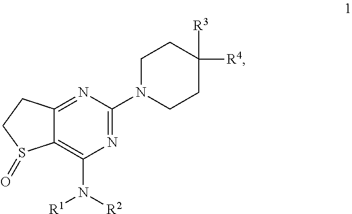
wherein:
R¹ is H or C₁₋₆-alkyl,
R² is H or C₁₋₁₀-alkyl or C₂₋₆-alkenyl, each optionally substituted by one or more groups selected from halogen and C₁₋₃-fluoroalkyl or optionally substituted by one or more groups selected from OR^{2.1}, COOR^{2.1}, CONR^{2.2}R^{2.3}, SR^{2.1}, SO—R^{2.1}, SO₂—R^{2.1}, C₆₋₁₀-aryl, het, hetaryl, a mono- or bicyclic —C₃₋₁₀-cycloalkyl, CH₂—NR^{2.2}R^{2.3}, and NR^{2.2}R^{2.3}, which in turn are optionally substituted by one or more groups selected from OH, halogen, OR^{2.1}, oxo, CF₃, CHF₂, CH₂F, C₁₋₆-alkyl, C₁₋₆-alkanol, C₆₋₁₀-aryl, COOR^{2.1}, CH₂—NR^{2.2}R^{2.3}, and NR^{2.2}R^{2.3},
R² is a mono- or polycyclic C₃₋₁₀ cycloalkyl optionally bridged one or more times via C₁₋₃-alkyl groups and optionally substituted by a group selected from branched or unbranched C₁₋₆-alkanol, C₁₋₃-fluoroalkyl, C₁₋₃-alkylene-OR^{2.1}, OR^{2.1}, COOR^{2.1}, —SO₂—NR^{2.2}R^{2.3}, het, —NH—CO—O—(C₁₋₆-alkyl), —NH—CO—(C₁₋₆-alkyl), —NH—CO—O—(C₆₋₁₀-aryl), —NH—CO—(C₆₋₁₀-aryl), —NH—CO—O-hetaryl, —NH—CO-hetaryl, —NH—CO—O—(C₁₋₃-alkylene)-(C₆₋₁₀-aryl), —NH—CO—(C₁₋₃-alkylene)-(C₆₋₁₀-aryl), —N(C₁₋₃-alkyl)-CO—(C₁₋₆-alkyl), —N(C₁₋₃-alkyl)-CO—O—(C₆₋₁₀-aryl), —N(C₁₋₃-alkyl)-CO—(C₆₋₁₀-aryl), —N(C₁₋₃-alkyl)-CO—O-hetaryl, —N(C₁₋₃-alkyl)-CO-hetaryl, —N(C₁₋₃-alkyl)-CO—O—(C₁₋₃-alkylene)-(C₆₋₁₀-aryl), —N(C₁₋₃-alkyl)-CO—(C₁₋₃-alkylene)-(C₆₋₁₀-aryl), C₆₋₁₀-aryl, C₁₋₆-alkyl, C₆₋₁₀-aryl-C₁₋₆-alkylene, hetaryl-C₁₋₆-alkylene, mono- or bicyclic C₃₋₁₀ cycloalkyl, and NR^{2.2}R^{2.3}, each optionally substituted by one or more groups selected from OH, OR^{2.1}, oxo, halogen, CF₃, CHF₂, CH₂F, C₁₋₆-alkyl, C₆₋₁₀-aryl, and NR^{2.2}R^{2.3},
R² is a mono- or polycyclic C₆₋₁₀-aryl optionally substituted by OH, SH, or halogen or by one or more groups selected from OR^{2.1}, COOR^{2.1}, NR^{2.2}R^{2.3}, CH₂—NR^{2.2}R^{2.3}, C₃₋₁₀-cycloalkyl, het, C₁₋₆-alkyl, C₁₋₃-fluoroalkyl, CF₃, CHF₂, CH₂F, C₆₋₁₀-aryl-C₁₋₆-alkylene, het-C₁₋₆-alkylene, hetaryl-C₁₋₆-alkylene, C₆₋₁₀-aryl, SO₂—CH₃, SO₂—CH₂CH₃ and SO₂—NR^{2.2}R^{2.3}, each optionally substituted by one or more groups selected from OH, OR^{2.1}, CF₃, CHF₂, CH₂F, oxo, halogen, CF₃, CHF₂, CH₂F, C₁₋₆-alkyl, C₆₋₁₀-aryl, and NR^{2.2}R^{2.3},
R² is het or hetaryl, each optionally substituted by one or more groups selected from halogen, OH, oxo, CF₃, CHF₂, and CH₂F or by one or more groups selected from OR^{2.1}, C₁₋₃-alkylene-OR^{2.1}, SR^{2.1}, SO—R^{2.1}, SO₂—R^{2.1}, COOR^{2.1}, COR^{2.1}, C₁₋₆-alkanol, mono- or bicyclic C₃₋₁₀-cycloalkyl, C₆₋₁₀-aryl, C₁₋₆-alkyl, C₆₋₁₀-aryl-C₁₋₆-alkylene, hetaryl-C₁₋₆-alkylene, het, hetaryl, C₁₋₃-alkylene-OR^{2.1}, and NR^{2.2}R^{2.3}, each optionally substituted by one or more groups selected from OH, OR^{2.1}, oxo, halogen, CF₃, CHF₂, CH₂F, C₁₋₆-alkyl, C₆₋₁₀-aryl, and NR^{2.2}R^{2.3}, or
NR¹R² together are an optionally bridged heterocyclic C₄₋₇ ring, which contains 1, 2, or 3 heteroatoms selected from N, O, and S, and optionally substituted by one or more groups selected from OH, OR^{2.1}, C₁₋₃-alkylene-O^{R.1}, oxo, halogen, C_{1 -6}-alkyl, C₆₋₁₀-aryl, COOR^{2.1}, CH₂—NR^{2.2}—COO—R^{2.1}, CH₂—NR^{2.2}—CO—R^{2.1}, CH₂—NR^{2.2}—CO—CH₂—NR^{2.2}R^{2.3}, CH₂—NR^{2.2}—SO₂—C₁₋₃-alkyl, CH₂—NR^{2.2}—SO₂—NR^{2.2}R^{2.3}, CH₂—NR^{2.2}—CO—NR^{2.2}R^{2.3}, CO—NR^{2.2}R^{2.3}, CH₂—NR^{2.2}R^{2.3}, and NR^{2.2}R^{2.3},
R³ is a C₆₋₁₀-aryl optionally substituted in the ortho, para, or meta position by one, two, or three groups independently selected from fluorine, chlorine, bromine, hydroxy, CN, C₁₋₆-alkyl, C₁₋₃-fluoroalkyl, —C₁₋₃-alkylene-OR^{2.1}, —C₁₋₃-alkylene-NR^{2.2}R^{2.3}, —NR^{2.2}R^{2.3}, O—R^{2.1}, SO—R^{2.1}, SO₂—R^{2.1}, COOR^{2.1}, —CO—NH—(C₁₋₆-alkylene)-hetaryl, —CO—NH-hetaryl, —CO—N(CH₃)-het, —CO—N(CH₃)—(C₁₋₃-alkylene)-het, —CO—N(CH₃)—(C₁₋₃-alkylene)-hetaryl, —CO—N(C₃₋₇-cycloalkyl)-het, —CO—NR^{2.2}R^{2.3}, —CO—NH—(C₁₋₆-alkylene)-het, NR^{2.2}—CO—R^{2.1}, C₆₋₁₀-aryl, C₆₋₁₀-aryl-C₁₋₂-alkylene, het-C₁₋₂-alkylene, het, —CO-het, CO—N(CH₃)—C₃₋₇-cycloalkyl, C₃₋₇-cycloalkyl, C₃₋₇-cycloalkyl-C₁₋₂-alkylene, hetaryl-C₁₋₂-alkylene, and hetaryl, each optionally substituted by one or more groups selected from OH, halogen, —C₁₋₃-fluoroalkyl, oxo, methyl, and phenyl,
R³ is het and hetaryl, each optionally substituted by one or more groups selected from halogen, C₁₋₃-fluoroalkyl, CN, OH, oxo, —C₁₋₆-alkyl, —C₁₋₃-alkylene-NR^{2.2}R^{2.3}, —NR^{2.2}R^{2.3}, SO—R^{2.1}, SO₂—R^{2.1}, —O—R^{2.1}, —COOR^{2.1}, SO₂—(CH₃), SO₂—(CH₂—CH₃), C₆₋₁₀-aryl, het, C₃₋₇-cycloalkyl, and hetaryl, each optionally substituted by one or more groups selected from OH, halogen, —C₁₋₃-fluoroalkyl, C₁₋₆-alkyl, C₆₋₁₀-aryl, —COO(C₁₋₃-alkyl), and O—(C₁₋₃-alkyl),
R³ is —O—R^{3.1}, wherein R^{3.1} is a group selected from —C₁₋₆-alkyl, —C₆₋₁₀-aryl, —C₁₋₃-alkylene-C₆₋₁₀-aryl, hetaryl, and het, each optionally substituted in the ortho, para, or meta position by one, two, or three groups independently selected from fluorine, chlorine, bromine, hydroxy, CN, C₁₋₆-alkyl, C₁₋₃-fluoroalkyl, CO—(C₁₋₅-alkyl), —CO—(C₁₋₃-fluoroalkyl), —CO—NH—(C₁₋₆-alkylene)-hetaryl, —CO—N(C₁₋₃-alkyl)-(C₁₋₆-alkylene)-hetaryl, —CO—N(C₁₋₃-alkyl)-het, —CO—N(C₃₋₇-cycloalkyl)-het, —C₁₋₃-alkylene-OR^{2.1}, —C₁₋₃-alkylene-NR^{2.2}R^{2.3}, —NR^{2.2}R^{2.3}, O—R^{2.1}, SO—R^{2.1}, SO₂—R^{2.1}, COOH, COO—(C₁₋₄-alkyl), —O—C₁₋₃-alkylene-N(C₁₋₃-alkyl)₂, CO—NR^{2.2}R^{2.3}, NR^{2.2}—CO—R^{2.1}, C₆₋₁₀-aryl, C₆₋₁₀-aryl-C₁₋₂-alkylene, het-C₁₋₂-alkylene, —CO-het, het, —CO—C₃₋₇-cycloalkyl, —CO—N(C₁₋₃-alkyl)-C₃₋₇-cycloalkyl, C₃₋₇-cycloalkyl, C₃₋₇-cycloalkyl-C₁₋₂-alkylene, hetaryl-C₁₋₂-alkylene, and hetaryl, each optionally substituted by 1, 2, 3, or 4 groups independently selected from F, Cl, Br, methyl, O-methyl, ethyl, O-ethyl, OH, oxo, and CF₃, and
R⁴ is H, CN, OH, CF₃, CHF₂, CH₂F, F, methyl, ethyl, —O—(C₁₋₃-alkyl), —C₁₋₃-alkylene-OH, —COO(C₁₋₃-alkyl), —CO-het, —(C₁₋₂-alkylene)-NH—SO₂—(C₁₋₂-alkyl), —(C₁₋₂-alkylene)-N(C₁₋₃-alkyl)-SO₂—(C₁₋₂-alkyl), —(C₁₋₂-alkylene)-O—(C₁₋₂-alkylene)-C₆₋₁₀-aryl, —C₁₋₃-alkylene-O—C₁₋₃-alkyl, —(C₁₋₂-alkylene)-N(C₁₋₃-alkyl)-CO—(C₁₋₂-alkyl), —NH—CO—(C₁₋₃-alkylene)-O—(C₁₋₃-alkyl), —C₁₋₃-alkylene-NH—CO—(C₁₋₃-alkyl), —C₁₋₃-alkylene-NH—CO—(C₁₋₃-alkylene)-N(C₁₋₃-alkyl)₂, —O—(C₁₋₂-alkylene)-(C₆₋₁₀-aryl), —C₁₋₃-alkylene-NH—CO—(C₁₋₃-alkylene)-O—(C₁₋₃-alkyl), —CO—(C₆₋₁₀-aryl), or —(C₁₋₂-alkylene)-N(C₁₋₃-alkyl)-CO—(C₁₋₂-alkylene)-O—(C₁₋₃-alkyl), wherein the aryl thereof is optionally substituted by one or more groups selected from F, Cl, Br, methyl, ethyl, propyl, isopropyl, cyclopropyl, —O-methyl, —O-ethyl, —O-propyl, —O-isopropyl, —O-cyclopropyl, —OH, and CF₃, or
R³ and R⁴ together form a mono- or bicyclic, unsaturated, saturated or partially saturated heterocycle, which contains 1, 2 or 3 heteroatoms selected from N, O, and S, and is optionally substituted by one or more groups selected from halogen, OH, oxo, C₁₋₃-fluoroalkyl, CN, C₁₋₆-alkyl, —O—R^{2.1}—COOR^{2.1}, SO—R^{2.1}, SO₂—R^{2.1}, —C₁₋₃-alkylene-NR^{2.2}R^{2.3}, —NR^{2.2}R^{2.3}, C₆₋₁₀-aryl, C₃₋₇-cycloalkyl, het, and hetaryl,
wherein:
R^{2.1} is H or is a group selected from C₁₋₆-alkyl, C₁₋₆-alkanol, C₁₋₃-haloalkyl, mono- or bicyclic, —C₃₋₁₀-cycloalkyl, C₆₋₁₀-aryl-C₁₋₆-alkylene, hetaryl-C₁₋₆-alkylene, het-C₁₋₆-alkylene, C₃₋₁₀-cycloalkyl-C₁₋₆-alkylene, a mono- or bicyclic C₆₋₁₀-aryl, heteroaryl, and het, each optionally substituted by one or more groups selected from OH, O—(C₁₋₃-alkyl), halogen, C₁₋₆-alkyl, and C₆₋₁₀-aryl,
R^{2.2} and R^{2.3} are independently H or a group selected from C₁₋₆-alkyl, mono- or bicyclic C₃₋₁₀ cycloalkyl, C₆₋₁₀-aryl-C₁₋₆-alkylene, hetaryl-C₁₋₆-alkylene, mono- or bicyclic C₆₋₁₀-aryl, het, hetaryl, CO—NH₂, CO—NHCH₃, —CO—N(CH₃)₂, SO₂—(C₁-C₂-alkyl), CO—R^{2.1} and COOR^{2.1}, each optionally substituted by one or more groups selected from among OH, halogen, C₁₋₆-alkyl, C₆₋₁₀-aryl, and COOR^{2.1},
het is a three- to eleven-membered, mono- or bicyclic, saturated or partially saturated, optionally anellated or optionally bridged heterocycle which contains 1, 2, 3, or 4 heteroatoms independently selected from N, S, or O,
hetaryl is a five- to ten-membered, mono- or bicyclic, optionally anellated heteroaryl, which contains 1, 2, 3, or 4 heteroatoms selected independently from N, S, or O, and
cycloalkyl is saturated or partially saturated,
or a pharmacologically acceptable salt thereof.

2. The method according to claim 1, wherein:
R¹ is H,
R² is H or C₁₋₆-alkyl optionally substituted by one or more groups selected from F, Cl, CF₃, CHF₂, or CH₂F or optionally substituted by one or more groups selected from OR^{2.1}, COOR^{2.1}, CONR^{2.2}R^{2.3}, SR^{2.1}, SO—R^{2.1}, SO₂—R^{2.1}, phenyl, het, hetaryl, a monocyclic C₃₋₇-cycloalkyl, CH₂—NR^{2.2}R^{2.3}, and NR^{2.2}R^{2.3}, each optionally substituted by one or more groups selected from OH, F, Cl, Br, CF₃, CHF₂, CH₂F, OR^{2.1}, oxo, methyl, ethyl, propyl, isopropyl, methanol, ethanol, phenyl, COOR^{2.1}, CH₂—NR^{2.2}R^{2.3}, and NR^{2.2}R^{2.3},
R² is a monocyclic C₃₋₇ cycloalkyl optionally substituted by a group selected from C₁₋₂-alkanol, C₁₋₃-fluoroalkyl, C₁₋₃-alkylene-OR^{2.1}, OR^{2.1}, COOR^{2.1}, SO₂—NR^{2.2}R^{2.3}, het, —NH—CO—O-(phenyl), methyl, ethyl, propyl, isopropyl, phenyl, phenyl-C₁₋₂-alkylene, -hetaryl-C₁₋₂-alkylene, monocyclic C₃₋₇ cycloalkyl, and NR^{2.2}R^{2.3}, each optionally substituted by one or more groups selected from OH, OR^{2.1}, oxo, F, Cl, CF₃, CHF₂, CH₂F, methyl, ethyl, propyl, isopropyl, phenyl, and NR^{2.2}R^{2.3},
R² is a phenyl optionally substituted by OH, SH, F, Cl, or Br or by one or more groups selected from OR^{2.1}, COOR^{2.1}, NR^{2.2}R^{2.3}, CH₂—NR^{2.2}R^{2.3}, monocyclic C₃₋₇-cycloalkyl, het, methyl, ethyl, propyl, isopropyl, CF₃, CHF₂, CH₂F, phenyl-C₁₋₂-alkylene, het-C₁₋₂-alkylene, hetaryl-C₁₋₂-alkylene, phenyl, SO₂—CH₃, SO₂—CH₂CH₃, and SO₂—NR^{2.2}R^{2.3}, each optionally substituted by one or more groups selected from OH, OR^{2.1}, oxo, F, Cl, CF₃, CHF₂, CH₂F, methyl, ethyl, propyl, isopropyl, phenyl, and NR^{2.2}R^{2.3},
R² is het or hetaryl, each optionally substituted by one or more groups selected from F, Cl, OH, oxo, CF₃, CHF₂, and CH₂F or by one or more groups selected from OR^{2.1}, C₁₋₃-alkylene-OR^{2.1}, sR^{2.1}, SO—R^{2.1}, SO₂—R^{2.1}, COOR^{2.1}, COR^{2.1}, methanol, ethanol, monocyclic C₃₋₇-cycloalkyl, phenyl, methyl, ethyl, propyl, isopropyl, phenyl-C₁₋₂-alkylene, hetaryl-C₁₋₂-alkylene, het, hetaryl, and NR^{2.2}R^{2.3}, each optionally substituted by one or more groups selected from OH, OR^{2.1}, oxo, F, Cl, CF₃, CHF₂, CH₂F, methyl, ethyl, propyl, isopropyl, butyl, isobutyl, phenyl, and NR^{2.2}R^{2.3},
R³ is a naphthalene or phenyl, each optionally substituted in the ortho, para, or meta position by one or two groups selected independently from fluorine, chlorine, bromine, hydroxy, CN, methyl, ethyl, propyl, isopropyl, cyclopropyl, CF₃, CHF₂, CH₂F, —OCH₃, OCH₂CH₃; SO₂—CH₃, SO—CH₃, COOCH₃, COOCH₂CH₃, —CO—NH-(methylene)-hetaryl, —CO—NH-(ethylene)-hetaryl, —CO—NH-hetaryl, —CO—N(CH₃)-het, —CO—N(CH₃)-(methylene)-het, —CO—N(CH₃)-(ethylene)-het, —CO—N(CH₃)-(methylene)-hetaryl, —CO—N(CH₃)-(ethylene)-hetaryl, —CO—N(cyclopropyl)-het, CO—NH₂, CONH(CH₃), CON(CH₃)₂, —CO—NH-(methylene)-het, —CO—NH-(ethylene)-het, —NH—CO-methyl, NCH₃—CO-methyl, —NH—CO-ethyl, NCH₃—CO-ethyl, —NH—CO-propyl, NCH₃—CO-propyl, —NH—CO-isopropyl, NCH₃—CO-isopropyl, phenyl, phenyl-methylene, phenyl-ethylene, het-methylene, het-ethylene, het, —CO-het, —CO—N(CH₃)-het, CO—N(CH₃)-cyclopropyl, C₃₋₇-cycloalkyl, C₃₋₇-cycloalkyl-methylene, C₃₋₇-cycloalkyl-ethylene, hetaryl-methylene, hetaryl-ethylene, hetaryl, CH₂—NH₂, CH₂—NH(CH₃), CH₂—N(CH₃)₂, —NH₂, —NH(CH₃), and —N(CH₃)₂, each optionally substituted by one or more groups selected from OH, F, Cl, —CF₃, CHF₂, CH₂F, oxo, methyl, and phenyl,
R³ is het or hetaryl, each optionally substituted by one or more groups selected from F, Cl, Br, CF₃, CHF₂, CH₂F, CN, OH, oxo, methyl, ethyl, propyl, isopropyl, butyl, isobutyl, cyclopropyl, —O-methyl, —O-ethyl, —O-propyl, —O-isopropyl, —COO-methyl, —COO-ethyl, —COO-propyl, —COO-isopropyl, SO—(CH₃), SO—(CH₂—CH₃), SO₂—(CH₃), SO₂—(CH₂—CH₃), phenyl, CH₂—NH₂, CH₂—NH(CH₃), CH₂—N(CH₃)₂, —NH₂, —NH(CH₃), —N(CH₃)₂, het, and hetaryl, each optionally substituted by one or more groups selected from OH, F, Cl, CF₃, CHF₂, CH₂F, methyl, ethyl, propyl, isopropyl, phenyl, —COO-methyl, —COO-ethyl, O-methyl, and O-ethyl,
R³ is —O—R^{3.1}, wherein R^{3.1} is a group selected from —C₁₋₃-alkyl, phenyl, —C₁₋₃-alkylene-phenyl, hetaryl, and het, each optionally substituted in the ortho, para, or meta position by one, two, or three groups independently selected from fluorine, chlorine, bromine, hydroxy, CN, methyl, ethyl, propyl, isopropyl, butyl, isobutyl, CF₃, CHF₂, CH₂F, CO-(methyl), CO-(ethyl), CO-(propyl), CO-(isopropyl), —CO—(CF₃), —CO—NH-(methylene)-hetaryl, —CO—NH-(ethylene)-hetaryl, —CO—N(CH₃) -(methylene)-hetaryl, —CO—N(CH₃)-(ethylene)-hetaryl, —CO—N(CH₃)-(propylene)-hetaryl, —CO—N(CH₃)-(isopropylene)-hetaryl-CO—N(CH₃)-het, —CO—N(cyclopropyl)-het, —CO—N(C₅₋₇-cycloalkyl)-het, -methylene-O-methyl, -ethylene-O-methyl, -propylene-O-methyl, -methylene-O-ethyl, -ethylene-O-ethyl, -propylene-O-ethyl, -methylene-NH₂, -methylene-NHCH₃, -methylene-N(CH₃)₂, -ethylene-NH₂, -ethylene-NHCH₃, -ethylene-N(CH₃)₂, NH₂, N(CH₃)₂, NHCH₃, —O-methyl, O-ethyl, O-propyl, O-isopropyl, O-butyl, O-isobutyl, —SO—CH₃, SO-ethyl, —SO-propyl, —SO-isopropyl, SO₂-methyl, —SO₂-ethyl, SO₂-propyl, SO₂-isopropyl, COOH, COO-(methyl), COO-(ethyl), COO-(propyl), COO-(isopropyl), —O-methylene-N(methyl)₂, —O-ethylene-N(methyl)₂, —O-methylene-N(ethyl)₂, —O-ethylene-N(ethyl)₂, CO—NH₂, CO—NH(CH₃), CO—N(CH₃)₂, —NH—CO-methyl, —NCH₃—CO-methyl, —NH—CO-ethyl, NCH₃—CO-ethyl, phenyl, phenyl-methylene, phenyl-ethylene, het-methylene, het-ethylene, —CO-het, het, —CO—C₅₋₇-cycloalkyl, —CO-cyclopropyl, —CO—N(CH₃)—C₅₋₇-cycloalkyl, CO—N(CH₃)-cyclopropyl, C₅₋₇-cycloalkyl, cyclopropyl, C₅₋₇-cycloalkyl-methylene, C₅₋₇-cycloalkyl-ethylene, cyclopropyl-methylene, cyclopropyl-ethylene, hetaryl-methylene, hetaryl-ethylene and hetaryl, which in turn may optionally be substituted by 1, 2, 3, or 4 groups selected independently of one another from F, Cl, Br, methyl, O-methyl, ethyl, O-ethyl, OH, oxo, and CF₃, and
R⁴ is H, CN, OH, CF₃, CHF₂, CH₂F, F, methyl, ethyl, O-methyl, O-ethyl, -methylene-OH, -ethylene-OH, -propylene-OH, isopropylene-OH, —COO(methyl), —COO(ethyl), —COO(propyl), —COO(isopropyl), —CO-het, -(methylene)-NH—SO₂-(methyl), -(methylene)-NH—SO₂-(ethyl), -(ethylene)-NH—SO₂-(methyl), -(ethylene)-NH—SO₂-(ethyl), -(methylene)-N(CH₃)—SO₂-(methyl), -(methylene)-N(CH₃)—SO₂-(ethyl), -(ethylene)-N(CH₃)—SO₂-(methyl), -(ethylene)-N(CH₃)—SO₂-(ethyl), -(methylene)-O-(methylene)-phenyl, -(methylene)-O-(ethylene)-phenyl, -(ethylene)-O-(methylene)-phenyl, -(ethylene)-O-(ethylene)-phenyl, -methylene-O-methyl, -methylene-O-ethyl, -ethylene-O-methyl-ethylene-O-ethyl, -(methylene)-N(CH₃)—CO-(methyl), -(methylene)-N(CH₃)—CO-(ethyl)-(ethylene)-N(CH₃)—CO-(methyl), -(ethylene)-N(CH₃)—CO-(ethyl), —NH—CO-(methylene)-O-(methyl), —NH—CO-(methylene)-O-(ethyl), —NH—CO-(ethylene)-O-(methyl), —NH—CO-(ethylene)-O-(ethyl), -methylene-NH—CO-(methyl), -methylene-NH—CO-(ethyl), -ethylene-NH—CO-(methyl), -ethylene-NH—CO-(ethyl), -methylene-NH—CO-(methylene)-N(methyl)₂, -methylene-NH—CO-(ethylene)-N(methyl)₂, -ethylene-NH—CO-(methylene)-N(methyl)₂, -ethylene-NH—CO-(ethylene)-N(methyl)₂, -methylene-NH—CO-(methylene)-O-(methyl), -methylene-NH—CO-(ethylene)-O-(methyl), -ethylene-NH—CO-(methylene)-O-(methyl), -methylene-NH—CO-(methylene)-O-(ethyl), -methylene-NH—CO-(ethylene)-O-(ethyl), -ethylene-NH—CO-(methylene)-O-(ethyl), -(methylene)-N(CH₃)—CO-(methylene)-O-(methyl), -(methylene)-N(CH₃)—CO-(ethylene)-O-(methyl), -(ethylene)-N(CH₃)—CO-(methylene)-O-(methyl), -(methylene)-N(CH₃)—CO-(methylene)-O-(ethyl), -(methylene)-N(CH₃)—CO-(ethylene)-O-(ethyl), -(ethylene)-N(CH₃)—CO-(methylene)-O-(ethyl), —O-(methylene)-phenyl, —O-(ethylene)-phenyl, —CO-phenyl, wherein the phenyl thereof is optionally substituted by one or more groups selected from F, Cl, Br, methyl, ethyl, propyl, —O-methyl, —O-ethyl, —O-propyl, —OH, and CF₃, or
R³ and R⁴ together form a mono- or bicyclic, unsaturated, saturated or partly saturated heterocyclic group which contains 1, 2, or 3 heteroatoms selected from N, O, and S, and is optionally substituted by one or more groups selected from F, Cl, Br, OH, oxo, CF₃, CHF₂, CH₂F, CN, methyl, ethyl, propyl, isopropyl, cyclopropyl, COO-methyl, —COO-ethyl, O-methyl, O-ethyl, SO₂—(CH₃), SO₂—(CH₂CH₃), SO—(CH₃), SO—(CH₂CH₃), CH₂—NH₂, CH₂—NH(CH₃), CH₂—N(CH₃)₂, —NH₂, —NH(CH₃), —N(CH₃)₂, phenyl, C₅₋₇-cycloalkyl, het, and hetaryl,
wherein:
R^{2.1} is H or a group selected from methyl, ethyl, propyl, isopropyl, methanol, ethanol, monocyclic C₃₋₇ cycloalkyl, phenyl-C₁₋₂-alkylene, -hetaryl-C₁₋₂-alkylene, -het-C₁₋₂-alkylene, C₃₋₇-cycloalkyl-C₁₋₂-alkylene, phenyl, hetaryl, and het, each optionally substituted by one or more groups selected from OH, F, Cl, methyl, ethyl, propyl, isopropyl, O-methyl, O-ethyl, O-propyl, O-isopropyl, and phenyl,
R^{2.2} and R^{2.3} are each independently H or a group selected from methyl, ethyl, propyl, isopropyl, monocyclic C₃₋₇-cycloalkyl, phenyl-C₁₋₃-alkylene, hetaryl-C₁₋₃-alkylene, phenyl, het, hetaryl, CO—NH₂, CO—NHCH₃, CON(CH₃)₂, SO₂—(C₁₋₂-alkyl), CO—R^{2.1}, and COOR^{2.1}, each optionally substituted by one or more groups selected from OH, F, Cl, methyl, ethyl, propyl, isopropyl, phenyl, and COOR^{2.1},
het is a three- to seven-membered, monocyclic, saturated or partly saturated heterocyclic group which contains 1, 2, or 3 heteroatoms independently selected from N, S, or O, and
hetaryl is a five- to six-membered, monocyclic, aromatic heteroaryl which contains 1, 2, or 3 heteroatoms independently selected from N, S, or O, or
a pharmacologically acceptable salt thereof.

3. The method according to claim 1, wherein:
R² is a group according to formula 3 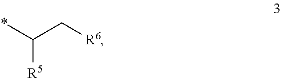
R⁵ is methyl, ethyl, propyl, or isopropyl, and
R⁶ is OH or NH₂, or
a pharmacologically acceptable salt thereof.

4. The method according to claim 1, wherein:
R² is a cyclopropyl or cyclobutyl, each optionally substituted by a group selected from OH, —CH₂—OH, —NH₂, CH₂—NH₂, —NH(CH₃), —N(CH₃)₂, methyl, ethyl, propyl, isopropyl, —NH—CO-(tert-butyl), —NH—CO—O-(tert-butyl), —N(CH₃)—CO-(tert-butyl), —N(CH₃)—CO—O-(tert-butyl), —CF₃, —CHF₂, CH₂F, F, Cl, and Br, or
a pharmacologically acceptable salt thereof.

5. The method according to claim 1, wherein:
R² is a phenyl optionally substituted in one or both meta positions by one or more groups selected from methyl, ethyl, propyl, isopropyl, cyclopropyl, F, Cl, Br, OH, OR^{2.1}, COOR^{2.1}, CF₃, CHF₂, CH₂F, NH₂, NH(CH₃), and N(CH₃)₂, wherein R^{2.1} is H, methyl, or ethyl, or
a pharmacologically acceptable salt thereof.

6. The method according to claim 1, wherein:
R² is piperidine or tetrahydropyran, each optionally substituted by one or more groups selected from F, Cl, Br, OH, CF₃, CHF₂, CH₂F, NH₂, NHCH₃, N(CH₃)₂, oxo, methyl, and methoxy, or
a pharmacologically acceptable salt thereof.

7. The method according to claim 1, wherein the compound of formula I is selected from the group consisting of:
1.1 (R)-2-{2-[4-(4-chlorophenyl)-piperidin-1-yl]-5-oxo-6,7-dihydro-5H-5λ⁴-thieno[3,2-d]pyrimidin-4-ylamino}-3-methylbutan-1-ol
1.2 (1-{2-[4-(4-chlorophenyl)-piperidin-1-yl]-5-oxo-6,7-dihydro-5H-5λ⁴-thieno[3,2-d]pyrimidin-4-ylamino}-cyclopropyl)-methanol
1.3 (R)-2-{2-[4-(4-chlorophenyl)-piperidin-1-yl]-5-oxo-6,7-dihydro-5H-5λ⁴-thieno[3,2-d]pyrimidin-4-ylamino}-pentan-1-ol
1.4 (R)-1-{2-[4-(4-chlorophenyl)-piperidin-1-yl]-5-oxo-6,7-dihydro-5H-5λ⁴-thieno[3,2-d]pyrimidin-4-ylamino}-1-(4-fluorophenyl)-2-methylpropan-2-ol
1.5 (S)-5-{2-[4-(4-chlorophenyl)-piperidin-1-yl]-5-oxo-6,7-dihydro-5H-5λ⁴-thieno[3,2-d]pyrimidin-4-ylamino}-1-methylpiperidin-2-one
1.6 {2-[4-(4-chlorophenyl)-piperidin-1-yl]-5-oxo-6,7-dihydro-5H-5λ⁴-thieno[3,2-d]pyrimidin-4-yl}-(tetrahydropyran-4-yl)-amine
1.7 1-(4-(1-hydroxymethylcyclopropylamino)-5-oxo-6,7-dihydro-5H-5λ⁴-thieno[3,2-d]pyrimidin-2-yl)-3′-methyl-1′H-spiro[piperidin-4,4′-quinazolin]-2′(3′H)-one
1.8 {1-[2-(4-benzo[d]isoxazol-3-yl-piperidin-1-yl)-5-oxo-6,7-dihydro-5H-5λ⁴-thieno[3,2-d]pyrimidin-4-ylamino]-cyclopropyl}-methanol
1.9 (1-{2-[4-(2-ethyl-5-fluoro-1H-indol-3-yl)-piperidin-1-yl]-5-oxo-6,7-dihydro-5H-5λ⁴-thieno[3,2-d]pyrimidin-4-ylamino}-cyclopropyl)-methanol
1.10 1-[4-((S)-1-methyl-6-oxopiperidin-3-ylamino)-5-oxo-6,7-dihydro-5H-5λ⁴-thieno[3,2-d]pyrimidin-2-yl]-4-phenylpiperidin-4-carbonitrile
1.11 3′-methyl-1-(4-(tetrahydro-2H-pyran-4-ylamino)-5-oxo-6,7-dihydro-5H-5λ⁴-thieno[3,2-d]pyrimidin-2-yl)-1′H-spiro[piperidin-4,4′-quinazolin]-2′(3′H)-one
1.12 (3-fluorophenyl)-[5-oxo-2-(3,4,5,6-tetrahydro-2H-[4,4′]bipyridinyl-1-yl)-6,7-dihydro-5H-5λ⁴-thieno[3,2-d]pyrimidin-4-yl]-amine
1.13 {2-[4-(2-ethyl-5-fluoro-1H-indol-3-yl)-piperidin-1-yl]-5-oxo-6,7-dihydro-5H-5λ⁴-thieno[3,2-d]pyrimidin-4-yl}-(3-fluorophenyl)-amine
1.14 (1-{2-[4-(2,4-difluorophenyl)-piperidin-1-yl]-5-oxo-6,7-dihydro-5H-5λ⁴-thieno[3,2-d]pyrimidin-4-ylamino}-cyclopropyl)-methanol
1.15 {2-[4-(2,4-difluorophenyl)-piperidin-1-yl]-5-oxo-6,7-dihydro-5H-5λ⁴-thieno[3,2-d]pyrimidin-4-yl}-(tetrahydropyran-4-yl)-amine
1.16 (S)-5-[2-(4-benzoxazol-2-yl-piperidin-1-yl)-5-oxo-6,7-dihydro-5H-5λ⁴-thieno[3,2-d]pyrimidin-4-ylamino]-1-methylpiperidin-2-one
1.17 (1-{2-[4-(6-fluorobenzo[d]isoxazol-3-yl)-piperidin-1-yl]-5-oxo-6,7-dihydro-5H-5λ⁴-thieno[3,2-d]pyrimidin-4-ylamino}-cyclopropyl)-methanol
1.18 (1-{2-[4-(5-fluorobenzo[d]isoxazol-3-yl)-piperidin-1-yl]-5-oxo-6,7-dihydro-5H-5λ⁴-thieno[3,2-d]pyrimidin-4-ylamino}-cyclopropyl)-methanol
1.19 {2-[4-(5-furan-2-yl-2H-pyrazol-3-yl)-piperidin-1-yl]-5-oxo-6,7-dihydro-5H-5λ⁴-thieno[3,2-d]pyrimidin-4-yl}-(tetrahydropyran-4-yl)-amine
1.20 (3-fluorophenyl)-{5-oxo-2-[4-(3-pyridin-4-yl-[1,2,4]oxadiazol-5-yl)-piperidin-1-yl]-6,7-dihydro-5H-5λ⁴-thieno[3,2-d]pyrimidin-4-yl}-amine
1.21 (R)-3-methyl-2-{5-oxo-2-[4-(3-pyridin-4-yl-[1,2,4]oxadiazol-5-yl)-piperidin-1-yl]-6,7-dihydro-5H-5λ⁴-thieno[3,2-d]pyrimidin-4-ylamino}-butan-1-ol
1.22 (S)-5-{2-[4-(4-fluorophenoxy)-piperidin-1-yl]-5-oxo-6,7-dihydro-5H-5λ4-thieno[3,2-d]pyrimidin-4-ylamino}-1-methylpiperidin-2-one
1.23 (2-{4-[4-(4,5-dihydrooxazol-2-yl)-phenoxy]-piperidin-1-yl}-5-oxo-6,7-dihydro-5H-5λ⁴-thieno[3,2-d]pyrimidin-4-yl)-(tetrahydropyran-4-yl)-amine
1.24 4-{1-[5-oxo-4-(tetrahydropyran-4-ylamino)-6,7-dihydro-5H-5λ⁴-thieno[3,2-d]pyrimidin-2-yl]-piperidin-4-yloxy}-benzoic acid
1.25 2-(1-{2-[4-(4-chlorophenyl)-piperidin-1-yl]-5-oxo-6,7-dihydro-5H-5λ⁴-thieno[3,2-d]pyrimidin-4-ylamino}-cyclopropyl)-propan-2-ol
1.26 {2-[4-(5-tert-butyl-1-methyl-1H-indol-3-yl)-piperidin-1-yl]-5-oxo-6,7-dihydro-5H-5λ⁴-thieno[3,2-d]pyrimidin-4-yl}-(tetrahydropyran-4-yl)-amine
1.27 2-[4-(5-furan-2-yl-1-methyl-1H-pyrazol-3-yl)-piperidin-1-yl]-5-oxo-6,7-dihydro-5H-5λ⁴-thieno[3,2-d]pyrimidin-4-yl}-(tetrahydropyran-4-yl)-amine
1.28 (S)-5-(2-{4-[4-(4,5-dihydrooxazol-2-yl)-phenoxy]-piperidin-1-yl}-5-oxo-6,7-dihydro-5H-5λ⁴-thieno[3,2-d]pyrimidin-4-ylamino)-1-methylpiperidin-2-one
1.29 {2-[4-(5-furan-2-yl-2-methyl-2H-pyrazol-3-yl)-piperidin-1-yl]-5-oxo-6,7-dihydro-5H-5λ⁴-thieno[3,2-d]pyrimidin-4-yl}-(tetrahydropyran-4-yl)-amine
1.30 {2-[4-(1-methyl-1H-imidazo[4,5-c]pyridin-2-yl)-piperidin-1-yl]-5-oxo-6,7-dihydro-5H-5λ⁴-thieno[3,2-d]pyrimidin-4-yl}-(tetrahydropyran-4-yl)-amine
1.31 2-methoxy-N-{1-[5-oxo-4-(tetrahydropyran-4-ylamino)-6,7-dihydro-5H-5λ⁴-thieno[3,2-d]pyrimidin-2-yl]-4-phenylpiperidin-4-ylmethyl}-acetamide
1.32 N-cyclopropyl-N-methyl-4-{1-[5-oxo-4-(tetrahydropyran-4-ylamino)-6,7-dihydro-5H-5λ⁴-thieno[3,2-d]pyrimidin-2-yl]-piperidin-4-yl}-benzamide
1.33 N-cyclopropyl-N-methyl-4-{1-[5-oxo-4-(tetrahydropyran-4-ylamino)-6,7-dihydro-5H-5λ⁴-thieno[3,2-d]pyrimidin-2-yl]-piperidin-4-yloxy}-benzamide
1.34 {5-oxo-2-[4-(pyridin-4-yloxy)-piperidin-1-yl]-6,7-dihydro-5H-5λ⁴-thieno[3,2-d]pyrimidin-4-yl}-(tetrahydropyran-4-yl)-amine
1.35 {2-[4-(4-chlorophenoxy)-piperidin-1-yl]-5-oxo-6,7-dihydro-5H-5λ⁴-thieno[3,2-d]pyrimidin-4-yl}-(tetrahydropyran-4-yl)-amine
1.36 (S)-1-methyl-5-{2-[4-(5-methyl-4-phenyloxazol-2-yl)-piperidin-1-yl]-5-oxo-6,7-dihydro-5H-5λ⁴-thieno[3,2-d]pyrimidin-4-ylamino}-piperidin-2-one
1.37 (1-{2-[4-(5-methyl-4-phenyloxazol-2-yl)-piperidin-1-yl]-5-oxo-6,7-dihydro-5H-5λ⁴-thieno[3,2-d]pyrimidin-4-ylamino}-cyclopropyl)-methanol
1.38 (S)-5-{2-[4-(4,5-diphenyloxazol-2-yl)-piperidin-1-yl]-5-oxo-6,7-dihydro-5H-5λ⁴-thieno[3,2-d]pyrimidin-4-ylamino}-1-methylpiperidin-2-one
1.39 {4-(4-chlorophenyl)-1-[5-oxo-4-(tetrahydropyran-4-ylamino)-6,7-dihydro-5H-5λ⁴-thieno[3,2-d]pyrimidin-2-yl]-piperidin-4-yl}-methanol
1.40 [1-(2-{4-[5-(4-chlorophenyl)-4-methyloxazol-2-yl]-piperidin-1-yl}-5-oxo-6,7-dihydro-5H-5λ⁴-thieno[3,2-d]pyrimidin-4-ylamino)-cyclopropyl-]methanol
1.41 4-(4-chlorophenyl)-1-[5-oxo-4-(tetrahydropyran-4-ylamino)-6,7-dihydro-5H-5λ⁴-thieno[3,2-d]pyrimidin-2-yl]-piperidin-4-ol
1.42 {2-[4-(4-chlorophenyl)-4-methoxypiperidin-1-yl]-5-oxo-6,7-dihydro-5H-5λ⁴-thieno[3,2-d]pyrimidin-4-yl}-(tetrahydropyran-4-yl)-amine
1.43 4-{1-[4-(1-hydroxymethylcyclopropylamino)-5-oxo-6,7-dihydro-5H-5λ⁴-thieno[3,2-d]pyrimidin-2-yl]-piperidin-4-yloxy}-benzonitrile
1.44 5-oxo-2-[4-(4,5,6,7-tetrahydrobenzoxazol-2-yl)-piperidin-1-yl]-6,7-dihydro-5H-5λ⁴-thieno[3,2-d]pyrimidin-4-yl}-(tetrahydropyran-4-yl)-amine
1.45 (S)-5-{2-[4-(4-chlorophenyl)-piperidin-1-yl]-5,5-dioxo-6,7-dihydro-5H-5λ⁶-thieno[3,2-d]pyrimidin-4-ylamino}-1-methylpiperidin-2-one
1.46 (1-{2-[4-(5-Chloro-pyrimidin-2-yl)-piperidin-1-yl]-5-oxo-6,7-dihydro-5H-5λ⁴-thieno[3,2-d]pyrimidin-4-yl-amino}-cyclobutyl)-methanol, and
1.47 (1-{2-[4-(4-Chloro-phenyl)-piperidin-1-yl]-5-oxo-6,7-dihydro-5H-5λ⁴-thieno[3.2-d]pyrimidin-4-yl-amino}-cyclobutyl)-methanol, or
a pharmacologically acceptable salt thereof.

8. The method according to claim 1, wherein the diabetes mellitus is diabetes mellitus type 1.

9. The method according to claim 1, wherein the diabetes mellitus is diabetes mellitus type 2.

10. The method according to claim 1, wherein the microvascular complication of diabetes mellitus is diabetic retinopathy, diabetic nephropathy, diabetic neuropathy, diabetic foot, and diabetic ulcer.

11. The method according to claim 1, wherein the microvascular complication of diabetes mellitus is myocardial infarct, acute coronary syndrome, unstable angina pectoris, stable angina pectoris, stroke, peripheral arterial occlusive disease, cardiomyopathy, heart failure, heart rhythm disorders, or vascular restenosis.

12. The method according to claim 1, wherein an additional active substance selected from metformin, sulphonylureas, nateglinide, repaglinide, thiazolidinediones, dipeptidylpeptidase 4 inhibitors (DPP4-inhibitors), peroxisome proliferator-activated receptor gamma agonists (PPAR-gamma-agonists), alpha-glucosidase inhibitors, insulin, insulin analogues, glucagon-like-peptide 1 (GLP-1), and glucagon-like-peptide 1 analogues (GLP1-analogues) is administered to the patient.
